(19) United States
(12) Patent Application Publication
(10) Pub. No.: US 2023/0190745 A1
(43) Pub. Date: Jun. 22, 2023

(54) USE OF METABOLIC REGULATORS FOR THE TREATMENT OF COVID-19

(71) Applicant: YISSUM RESEARCH DEVELOPMENT COMPANY OF THE HEBREW UNIVERSITY OF JERUSALEM LTD, Jerusalem (IL)

(72) Inventors: Yaakov NAHMIAS, Mevaseret Zion (IL); Avner EHRLICH, Jerusalem (IL)

(73) Assignee: YISSUM RESEARCH DEVELOPMENT COMPANY OF THE HEBREW UNIVERSITY OF JERUSALEM LTD., Jerusalem (IL)

(21) Appl. No.: 17/965,150

(22) Filed: Oct. 13, 2022

Related U.S. Application Data
(63) Continuation of application No. PCT/IL2021/050422, filed on Apr. 13, 2021.
(60) Provisional application No. 63/009,270, filed on Apr. 13, 2020.
(60) Provisional application No. 63/026,330, filed on May 18, 2020.

(51) Int. Cl. A61K 31/505; A61K 31/216; A61K 31/195; A61K 31/201; A61P 31/14

(52) U.S. Cl. CPC A61K 31/505; A61K 31/216; A61K 31/195; A61K 31/201; A61P 31/14

(57) ABSTRACT

Methods of treating coronavirus infection, or decreasing the risk of symptomatic infection, by administering a peroxisome proliferator-activated receptor alpha (PPARA) agonist or an inositol-requiring enzyme 1 (IRE1) pathway inhibitor are provided.

### CROSS REFERENCE TO RELATED APPLICATIONS

This application is a Bypass Continuation of PCT Patent Application No. PCT/IL2021/050422 having International filing date of Apr. 13, 2021, which claims the benefit of priority of U.S. Provisional Application Nos. 63/009,270, filed Apr. 13, 2020 and 63/026,330, filed May 18, 2020 both titled “USE OF METABOLIC REGULATORS FOR THE TREATMENT OF COVID-19” the contents of which are all incorporated herein by reference in their entirety.

### REFERENCE TO AN ELECTRONIC SEQUENCE LISTING

The contents of the electronic sequence listing (HUJI-P-071-US.xml; Size: 4,844 bytes; and Date of Creation: Oct. 13, 2022) is herein incorporated by reference in its entirety.

### FIELD OF INVENTION

The present invention is in the field of treatment of viral infection.

### BACKGROUND OF THE INVENTION

The severe acute respiratory syndrome coronavirus 2 (SARS-CoV-2) is a positive-strand RNA virus of the Sarbecovirus subgenus that is related to SARS and MERS. SARS-CoV-2 infection leads to the development of coronavirus disease (COVID-19) an inflammatory lung condition resulting in acute respiratory distress and organ failure. SARS-CoV-2 has infected over 70 million individuals worldwide causing more than 1.6 million deaths in less than a year since its emergence. Like other viruses, SARS-CoV-2 relies on host machinery to propagate, rewiring cellular metabolism to generate macromolecules needed for virion replication, assembly, and egress.

Recent work suggests that COVID-19 progression is dependent on metabolic mechanisms. Elevated blood glucose, obesity, and hyperlipidemia were found to be risk factors for SARS-CoV-2 induced acute respiratory distress, independently from diabetes. In fact, metabolic risk factors are associated with a more than 3-fold increase in COVID-19 severity risk, while inflammatory lung diseases, such as chronic obstructive pulmonary disease (COPD), and asthma are associated with less than a 1.5-fold increase in risk.

Alarmingly, evidence from previous coronavirus outbreaks indicates that the metabolic rewiring induced by infection has detrimental and long-term effects post-recovery. MERS infection resulted in long term immune dysregulation and enhanced susceptibility for metabolic diseases, while SARS infection resulted in long-term alterations to lipid metabolism, hyperlipidemia, and hyperglycemia even 12 years post-recovery. Similar long-term effects of SARS-CoV-2 are possible post-recovery. Methods and compositions for treating SARS-CoV-2 that are effective and target the metabolic dysregulation caused by the disease are greatly needed.

### SUMMARY OF THE INVENTION

The present invention provides methods of treating coronavirus infection by administering a peroxisome proliferator-activated receptor alpha (PPARA) agonist or an inositol-requiring enzyme 1 (IRE1) pathway inhibitor.

According to a first aspect, there is provided a method of treating a coronavirus infection or preventing a symptomatic coronavirus infection in a subject in need thereof, the method comprising administering to the subject a therapeutic composition comprising at least one of a peroxisome proliferator-activated receptor alpha (PPARA) agonist and an inositol-requiring enzyme 1 (IRE1) pathway inhibitor, thereby treating a coronavirus infection or preventing a symptomatic coronavirus infection in a subject.

According to another aspect, there is provided a therapeutic composition comprising at least one of a peroxisome proliferator-activated receptor alpha (PPARA) agonist and an inositol-requiring enzyme 1 (IRE1) pathway inhibitor for use in treating a coronavirus infection or preventing a symptomatic coronavirus infection in a subject in need thereof.

According to some embodiments, the coronavirus is from the genus Betacoronavirus.

According to some embodiments, the coronavirus is for the subgenus Sarbecovirus.

According to some embodiments, the coronavirus is selected from Severe Acute Respiratory Syndrome (SARS)-CoV-1, Middle East Respiratory Syndrome (MERS) and SARS-CoV-2.

According to some embodiments, the coronavirus is SARS-CoV-2.

According to some embodiments, the subject is a mammal.

According to some embodiments, the administering is within 1 day of diagnosis of the infection.

According to some embodiments, the subject has not yet reached a cytokine storm stage of the infection.

According to some embodiments, the subject is not currently or was not previously treated with a PPARA agonist or IRE1 pathway inhibitor.

According to some embodiments, the subject does not suffer from a metabolic disease or disorder.

According to some embodiments, the PPARA agonist produces at least a 10-fold greater agonizing effect on PPARA than on PPAR gamma.

According to some embodiments, the PPARA agonist is selected from a fibrate, pirinixic acid and conjugated linoleic acid (CLA) and derivatives thereof.

According to some embodiments, the CLA is selected from 9-CLA and 10-CLA.

According to some embodiments, the fibrate is selected from aluminum clofibrate, bezafibrate, ciprofibrate, choline fenofibrate, clinofibrate, clofibrate, clofibride, fenofibrate, gemfibrozil, pemafibrate, fenofibric acid, ronifibrate and simfibrate.

According to some embodiments, the fibrate is fenofibrate.

According to some embodiments, the IRE1 pathway inhibitor is an IRE1 alpha (IRE1α) inhibitor.

According to some embodiments, the IRE1α inhibitor is selected from telmisartan, Sunitinib, STF-083010, 4 μ8C, KIRA6m, Kira8, Kira7, MKC8866, GSK2850163, Toyocamycin, APY29, MKC3946, MKC9989, NSC95682, B-I09, 3,6-DMAD, and IRE1α kinase-IN-2.

According to some embodiments, the administering is oral administering.

According to some embodiments, the PPARA agonist or IRE1 pathway inhibitor is formulated to reach a Cmax in the subject within 1 day from administration.

According to some embodiments, the PPARA agonist is formulated as a fenofibrate nanocrystal, optionally wherein the fenofibrate nanocrystal is selected from Tricor® and Triglide®.

According to some embodiments, the administering is intravenous administering.

According to some embodiments, the PPARA agonist or IRE1 pathway inhibitor is administered on the first day of administration at twice a dose administered for treating a metabolic condition and is subsequently administered at the dose for treating a metabolic condition.

According to some embodiments, the treating comprises at least one of reduced phospholipid accumulation in lung cells, reduced viral load, reduced symptoms, reduced inflammation, reduced risk of invasive mechanical ventilation, reduced risk of septic shock, reduced risk of acute liver injury, reduced risk of acute kidney injury, reduced risk of acute cardiac injury, reduced risk of ICU admission, reduced hospitalization time, reduced risk of Acute respiratory distress syndrome (ARDS), reduced risk of a cytokine storm and reduced risk of death.

According to some embodiments, the reduced inflammation is characterized by reduced levels of C-reactive protein (CRP).

According to some embodiments, the treating occurs within 5 days of administering.

According to some embodiments, the treating comprises treatment of post-acute sequelae of the coronavirus infection.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-G: Transcriptional metabolic signature of SARS-CoV-2 infection. (1A) Dot plot visualization of GO terms enriched by SARS-CoV-2 infection. COVID-19 patients samples include epithelial cells isolated by bronchoalveolar lavage (lavage) and post-mortem lung biopsies (autopsy). Culture samples include small airway epithelial cells (alveoli) and bronchial epithelial cells (bronchial) infected with SARS-CoV-2. Enrichment analysis shows the induction of immune response and inflammation as well as cellular stress (FDR<10-22) and lipid metabolism (FDR<10-5). (1B) Venn diagram describing the relationship between differentially expressed genes (DEG), metabolic genes (GO:0008152), and lipid metabolism genes (GO:0006629) in SARS-CoV-2 infection of primary bronchial epithelial cells and COVID-19 patient samples. Across all four sample groups 58±3% of the differentially expressed genes were metabolism-related, with 15±2% of the genes associated with lipid metabolism. (1C) Sunburst graph showing the coverage of composite metabolic terms on general metabolic response induced by SARS-CoV-2 infection of primary bronchial epithelial cells and COVID-19 patient samples. Lipid and mitochondrial metabolism dominate the transcriptional metabolic signature of infection across all four samples include bronchiole cells, small airway cells, patient tissue extracted by lavage and patient lung biopsies (see 1E-G). (1D) Schematic depicting the metabolic landscape of SARS-CoV-2 infection superimposed with a heat map of pathway-associated metabolic and stress genes. Red and green boxes are up and downregulated by infection, respectively. * marks differentially regulated genes (n=3, FDR<0.05). (1E) Venn diagrams describing the relationship between differentially expressed genes (DEG), metabolic genes (GO:0008152), and lipid metabolism genes (GO:0006629) in SARS-CoV-2 infection. Patient samples include epithelial cells isolated by bronchoalveolar lavage (lavage) and post-mortem lung biopsies (autopsy). Culture samples include small airway epithelial cells (alveoli) and bronchial epithelial cells (bronchial) infected with SARS-CoV-2. (1F) Sunburst graph showing the coverage of composite metabolic terms on general metabolic response induced by SARS-CoV-2 infection of primary bronchial epithelial cells and COVID-19 patient samples. (1G) Heat map of pathway-associated metabolic and stress genes across all four sample groups.

FIGS. 2A-K: Metabolic effect of SARS-CoV-2 infection. (2A) Schematic of ER stress pathways superimposed with pathway-associated genes. Red and green boxes are up and downregulated by infection, respectively. * marks differentially regulated genes (n=3, FDR<0.05). Red arrows note putative activation pathways based on the transcriptional response. XBP1S/U is ratio of IRE1-spliced over un-spliced form of XBP1. (2B) Schematic of central carbon metabolism fluxes superimposed with flux-associated genes. Differentially expressed genes (n=3, FDR <0.01) are marked with *. Genes and associated fluxes are highlighted red or green for up- or down-regulation, respectively. (2C) Metabolic analysis of SARS-CoV-2 and mock-infected primary bronchial epithelial cells confirms a 50% increase (n=6, p<0.001) in lactate production 48 hours post-infection. (2D) Ratio of lactate production to glucose uptake (glycolytic index) in SARS-CoV-2 and mock-infected primary cells. Index increases from 1.0 to 1.7 out of 2.0 marking complete conversion of glucose to lactate. (2E) Fluorescence images of primary bronchial epithelial cells infected with SARS-CoV-2 virus or mock control. Cells show 85% increase in accumulation of 2-NDBG a fluorescent glucose analog (n=3; Bar=40 μm). (2F) Schematic of lipid metabolism fluxes superimposed with flux-associated genes. Differentially expressed genes (n=3, FDR <0.01) are marked with *. Genes and associated fluxes are highlighted red or green for up- or down-regulation, respectively. (2G) Fluorescence images of primary bronchial epithelial cells infected with SARS-CoV-2 virus or mock control. Neutral lipids (triglycerides) are dyed green while phospholipids are dyed red. Image analysis shows a 23% increase in triglycerides (n=3, p<0.05) and % increase in phospholipids (n=3, p<0.001) following SARS-CoV-2 infection (Bar=10 μm). (2H) T-distributed stochastic neighbor embedding (tSNE) plot shows clustering of 2,629 cells based on gene expression. Point coordinates are based on the top 6 principal components calculated from the 5,784 most informative genes. Cell color specifies assignment to 1 of 6 clusters identified as virus replicating cells, as well as basal, secretory, FOXN4-positive, ciliated cell clusters. (2I) Expression of SARS-CoV-2 (nCoV) superimposed on the tSNE plot and associated abundance analysis showing epithelial cluster enriched with virus RNA. (2J) Gene enrichment analysis comparing virus replicating cells to uninfected epithelial cells showing differences in the cellular response to endoplasmic reticulum stress (FDR<1×10⁻⁶), metabolic processes (FDR<9×10⁻⁴) and lipid metabolism (FDR<3×10⁻³) in the same patients. (2K) Schematic of ER stress pathways superimposed with pathway-associated genes in virus replicating cluster. Red and green boxes are up and downregulated by infection, respectively. * marks differentially regulated genes (n=3, FDR<0.05). Red arrows note putative activation pathways based on the transcriptional response. XBP1S/U is ratio of IRE1-spliced over un-spliced form of XBP1.

FIGS. 3A-K: Viral protein modulation of metabolic pathways. Analysis of primary bronchial epithelial cells expressing different SARS-CoV-2 proteins for 72 hours using multiple independent assays. (3A) Analysis of ER stress markers CHOP and XBP1 splicing (XBP1S/U) by qRT-PCR. Expression of ORF9c, M, N, ORF3a, NSP7, ORFS, NSP5 and NSP12 significantly up regulated both markers (n=6, p<0.05). (3B) Fluorescence images showing neutral lipids (triglycerides) dyed as green lipid droplets while phospholipids are dyed red and showing a perinuclear abundance. (3C) Quantification of microscopic analysis of lipid accumulation showed no significant difference in triglyceride abundance, but significant accumulation of phospholipids in cells expressing the same panel of viral proteins that induced ER stress (n=6, p<0.01). (3D) Fluorescence images showing increased abundance of 2-NDBG a fluorescent glucose analog by a smaller set of viral proteins than above. (3E) Microscopic analysis shows significant increase in glucose accumulation in bronchial cells expressing N, ORF3a, NSP7, ORFS, NSP5 and NSP12 (n=6, p<0.05). (3F) Direct measurement of lactate production of bronchial epithelial cells. Cells expressing the same viral protein sub-set showed significantly higher lactate production (n=6, p<0.01). (3G) Ratio of lactate production to glucose uptake (glycolytic index) in bronchial cells expressing viral proteins. Index significantly increases from 1.1 to 1.7 marking a shift to glycolysis (n=6, p<0.01) induced by the viral proteins. (3H) Seahorse™ analysis of extracellular acidification rate (ECAR) surrogate measurement for lactate production, shows independent confirmation of increased glycolysis. (3I) Seahorse™ mitochondrial stress analysis of bronchial cells expressing the viral proteins. Oxygen consumption rate (OCR) is shown as a function of time. Oligomycin, FCCP, and antimycin/rotenone were injected at 25, 55 and 85 minutes, respectively. (3J) Quantification of oxidative phosphorylation (OXPHOS) shows decrease of mitochondrial function following expression of N, ORF3a and NSP7 (n=6, p<0.05). (3K) Bar chart showing analysis of ER stress markers CHOP, BiP, HERPUD1, EDEM1 and XBP1 splicing (XBP1S/U) by qRT-PCR in primary bronchial epithelial cells expressing different SARS-CoV-2 proteins (n=6). * p<0.05, ** p<0.01, *** p<0.001

FIGS. 4A-J: Emerging therapeutic targets of SARS-CoV-2 induced host metabolic pathways. (4A) Schematic depicting the metabolic landscape of SARS-CoV-2 infection superimposed with transcription factors found to regulate each pathway. Potential drugs (white boxes) and their therapeutic targets are marked on the chart. (4B) Table summarizing potential drugs, dietary supplements, and experimental molecules that can potentially reverse SARS-CoV-2 induced metabolic alterations. (4C) Microscopic analysis of lipid accumulation in SARS-CoV-2 infected lung cells exposed to different drugs for 5 days compared to DMSO-treated (vehicle) and mock-infected controls. Cells treated with PPARα agonist fenofibrate had lower phospholipid content, with neutral lipids packed in lipid droplets. Image-based quantification of neutral lipid and phospholipids. (4D) Lactate over glucose ratio of SARS-CoV-2 infected primary lung cells treated with various drugs. Fenofibrate significantly reduced the lactate to glucose ratio by 60% (p<0.01) normalizing the metabolic shift induced by infection (n=3; Bar=50 μm). (4E) Quantification of SARS-CoV-2 viral RNA over 5 days of treatment with various drugs or DMSO (vehicle). Treatment with 20 μM fenofibrate reduced SARS-CoV-2 viral load by 2-logs close to the detection limit of the assay (n=3; p<0.001). Treatment with 10 μM GW992 or 10 μM cloperastine reduced viral load by 2.5 to 3-fold (n=3; p<0.05). (4F) Bar chart summarizing microscopic analysis of cell viability in SARS-CoV-2 infected lung cells exposed to different drugs for 5 days compared to DMSO-treated (vehicle) and mock-infected controls. Cells treated with PPARα agonist fenofibrate had lower phospholipid content, with neutral lipids packed in lipid droplets. (4G) Bar charts summarizing a chi-squared test comparing the representation of patients taking different metabolic regulators in 249,939 general hospital medical records (medical records) compared to their representation in 1,531 confirmed COVID-19 cases over the same period. Patients taking fibrates (bezafibrate or ciprofibrate) were under-represented across all severity indicators, while those taking other metabolic regulators were over-represented in the population. * p<0.05, ** p<0.01, *** p<0.001. (4H) Comparative analysis of viral load and cell viability in SARS-CoV-2 infected lung cells exposed to different drugs for 5 days compared to DMSO-treated (vehicle) and mock-infected controls. (4I) Microscopic analysis of lipid accumulation in viral ORF3a expressing lung cells exposed to different PPARα agonists for 5 days compared to DMSO-treated (vehicle) and mock-infected controls. (4J) Flow diagram of the retrospective cohort assembled to compare patients hospitalized with COVID-19 taking different metabolic regulators.

FIGS. 5A-E: Metabolic regulation contributes to a differential immunoinflammatory response. (5A-C) Dynamic changes in circulating CRP levels, neutrophils, and lymphocytes in treatment and PSM-matched non-treatment groups (PSM-ctrl) during 21-day hospitalization. The centerline shows the mean value while the 95% confidence interval is represented by the shaded region. (5A) High CRP levels gradually declined in all PSM-matched control groups. Fibrates and IRE-inhibitor groups showed a significantly faster decline in inflammation, while the thiazolidinedione group did not appear to change. (5B) Neutrophil counts rose in all PSM-matched control groups above normal values (dotted red line). The fibrates group didn't show elevated neutrophil count, while the thiazolidinedione group rose significantly above its PSM-matched control. (5C) Lymphocyte counts failed to rise in all PSM-matched controls above the lower limit of normal (dotted red line). Fibrates and IRE-inhibitor groups showed significant elevation in lymphocytes above normal. (5D) 28-days survival curves of treatment and PSM-matched non-treatment groups (PSM-ctrl). Adjusted hazard ratio (HR) was calculated based on the mixed effect cox model with adjustment for age, gender, clinical characteristics on admission (heart rate, blood pressure, oxidation, and temperature), pre-existing comorbidities (smoking, asthma, COPD, diabetes, hypertension, diabetes, coronary heart disease, obesity, dyslipidemia, cerebrovascular disease, chronic liver disease, and chronic kidney disease) and indicators of disease severity and organ injuries on admission (platelets, neutrophil, lymphocyte counts, and CRP, cardiac troponin, ferritin, creatinine, LDH, d-dimer, bilirubin, lactic acid, and glucose levels). Mortality in fibrates and statins groups was significantly lower than control. (5E) Dynamic changes in composed immuno-inflammatory marker neutrophil to lymphocyte ratio and monocytes in treatment and PSM-matched non-treatment groups (PSM-ctrl) during 21-day hospitalization.

### DETAILED DESCRIPTION OF THE INVENTION

The present invention, in some embodiments, provides methods of treating coronavirus infection or reducing the risk of symptomatic infection by administering a peroxisome proliferator-activated receptor alpha (PPARA) agonist or an inositol-requiring enzyme 1 (IRE1) pathway inhibitor.

This is invention is based, at least in part, on the surprising finding that known metabolic response modulating drugs, in particular PPARα agonists and IREIα inhibitors, can be used to effectively treat COVID-19. The metabolic response of primary lung epithelial cells to SARS-CoV-2 infection was tracked, and induction of IRE1 and PKR/PERK pathways of endoplasmic stress were observed along with an associated increase in lipid accumulation, driven in part by inhibition of lipid catabolism, as well as Warburg-like effect. A comprehensive screen demonstrated a role for several viral proteins in mediating the metabolic response even in the absence of replication. Screening pharmaceutical modulators of each metabolic pathway showed that fenofibrate, a PPARα-agonist that induces lipid catabolism, reversed metabolic changes and blocked SARS-CoV-2 replication in vitro. Propensity score-matched (PSM) analysis of 1,531 patients hospitalized due to COVID-19 confirmed these in vitro observations. Patients taking fibrates showed significantly lower markers of immuno-inflammation and faster recovery, while those taking thiazolidinediones that simulate lipid synthesis showed sustained inflammation. Cox proportional-hazard analysis showed adjusted hazard ratio for 28-day mortality of 4×10⁻⁸ for fibrates, but higher mortality for patients taking thiazolidinediones, confirming a key role of lipid metabolism in the pathogenesis of COVID-19.

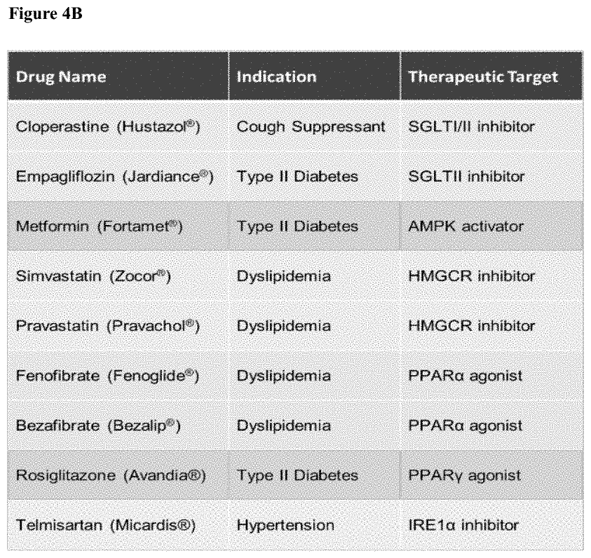

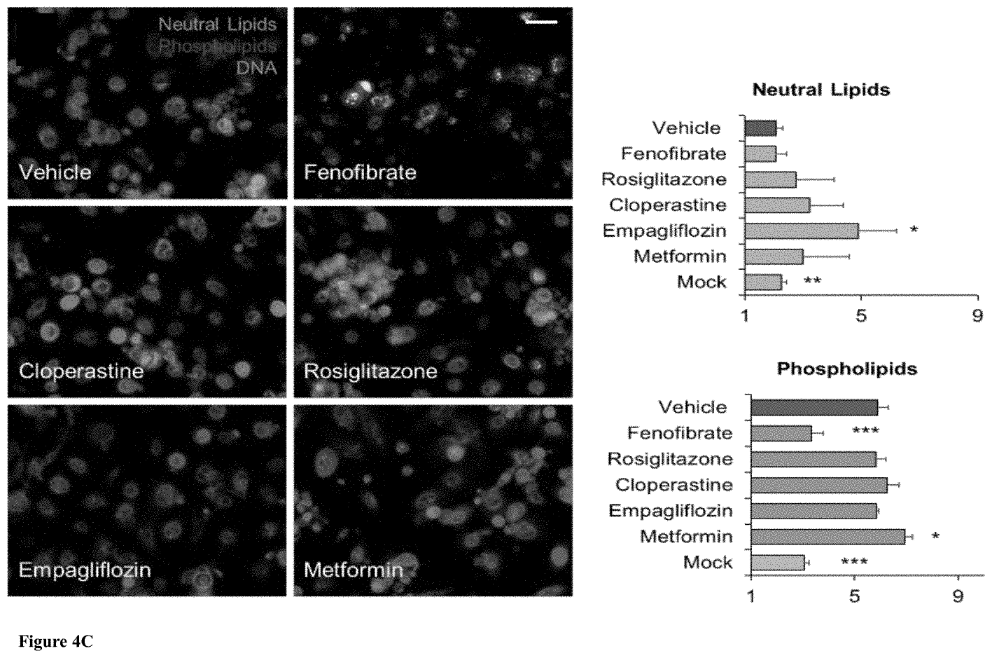

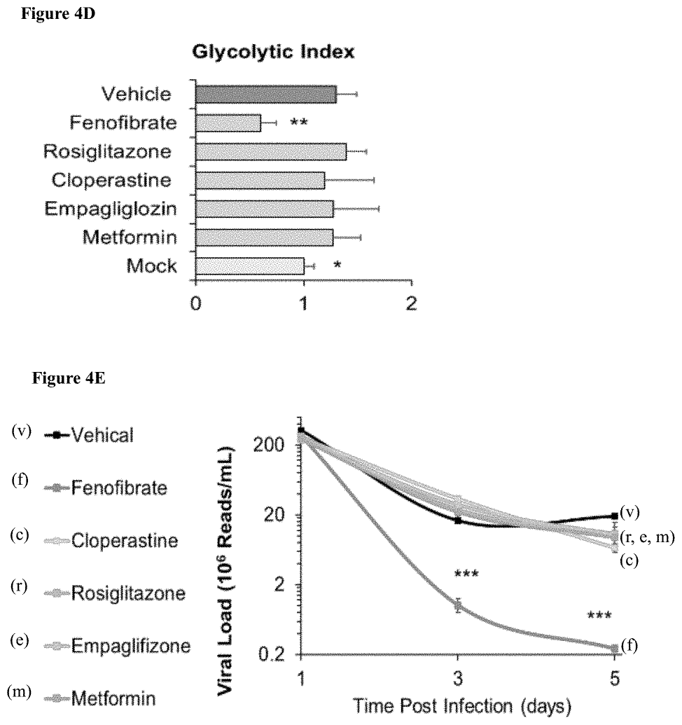

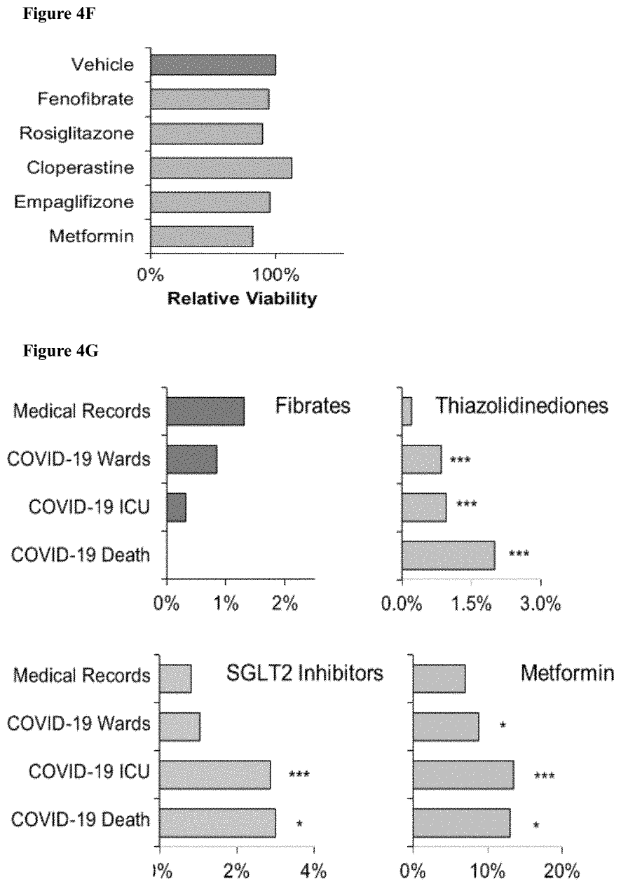

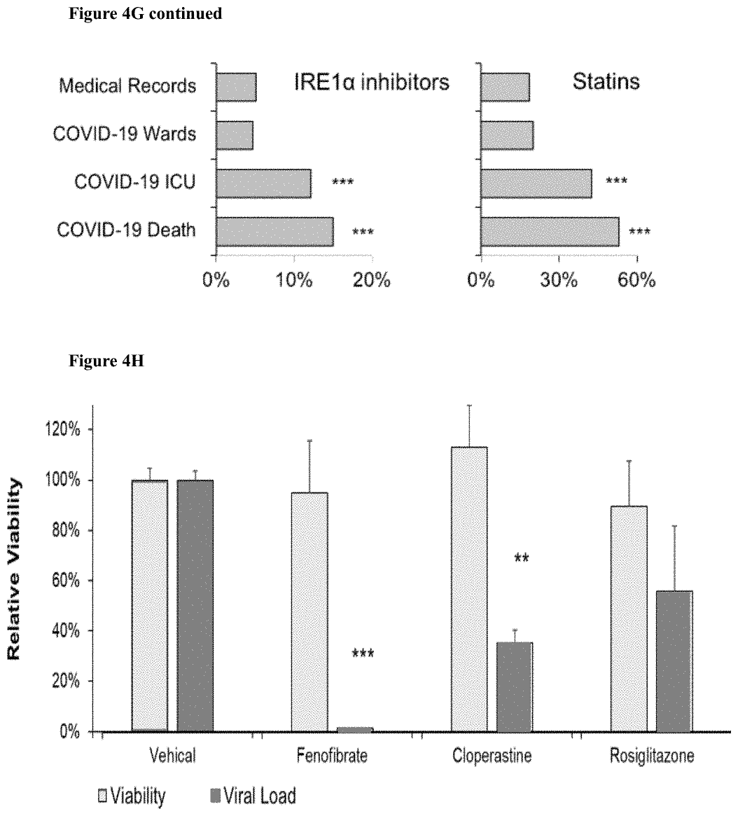

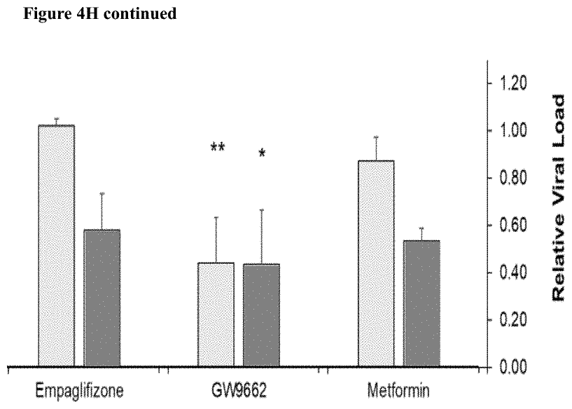

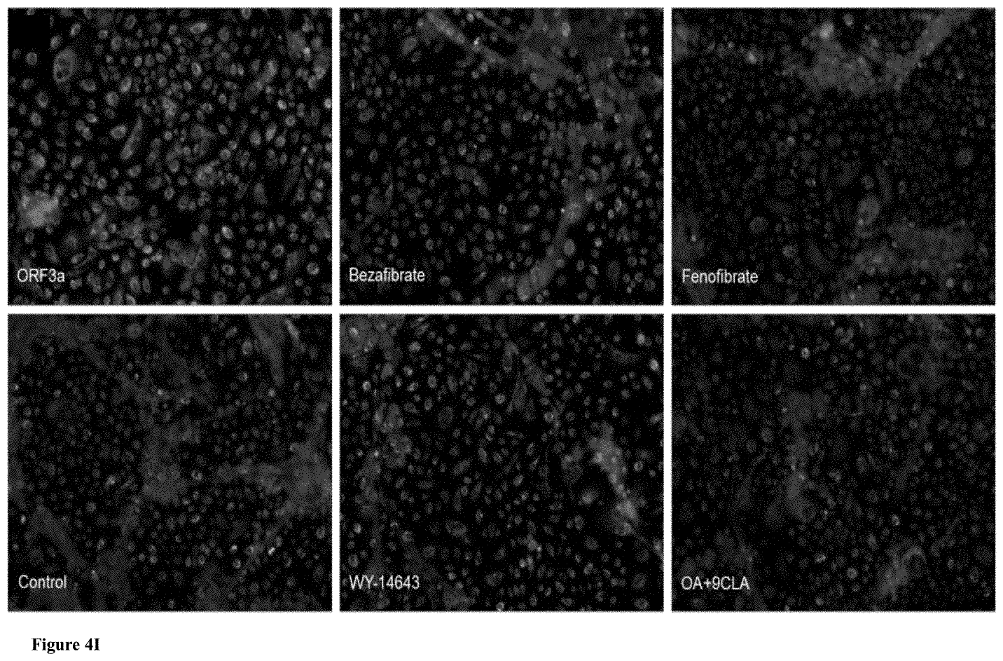

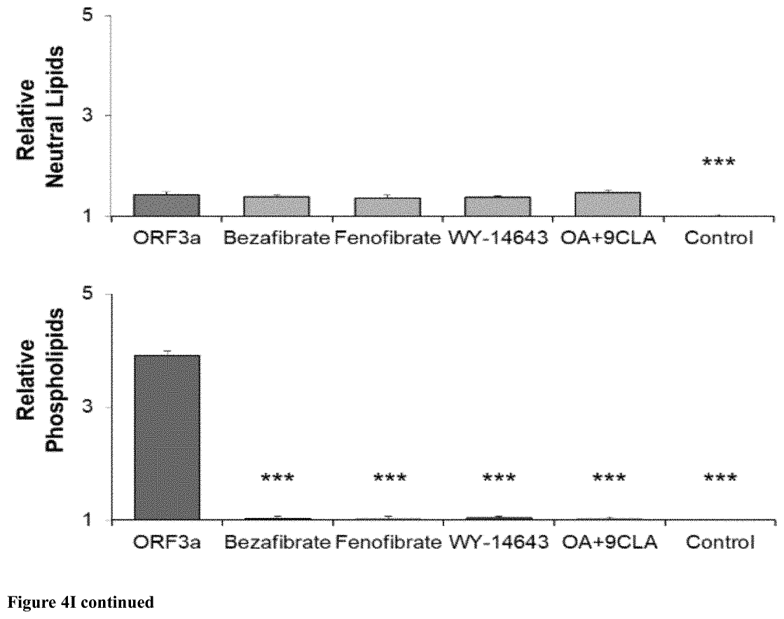

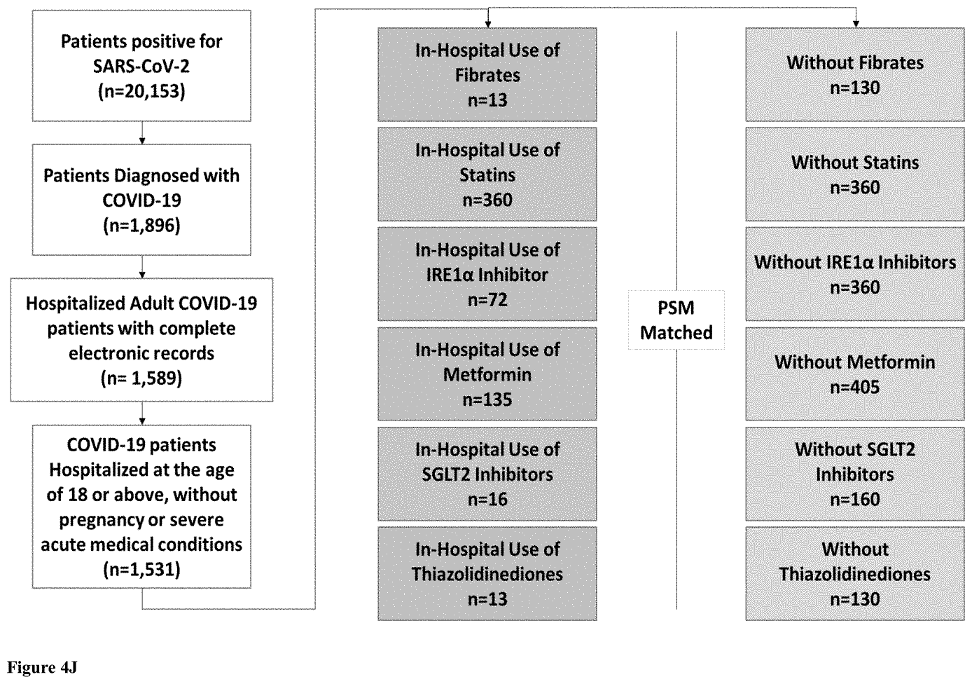

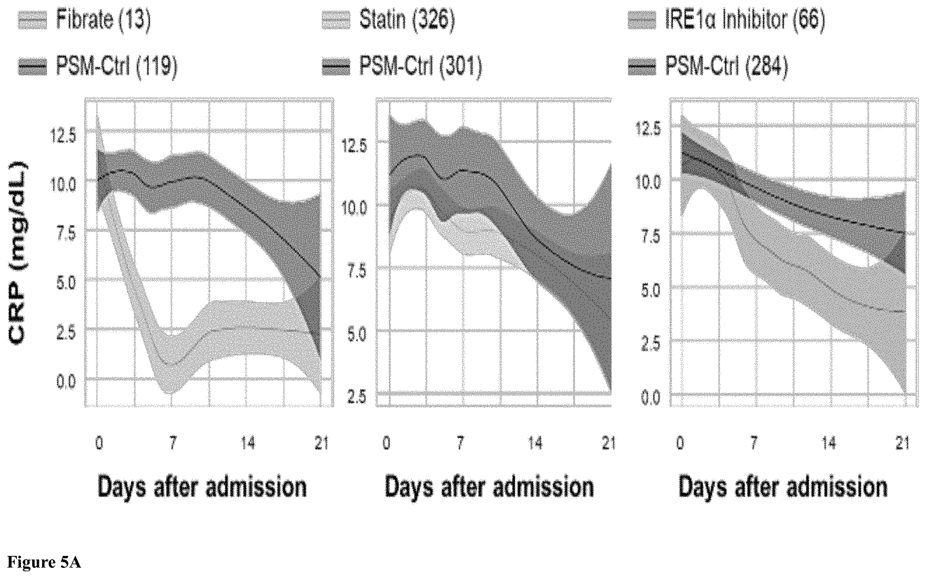

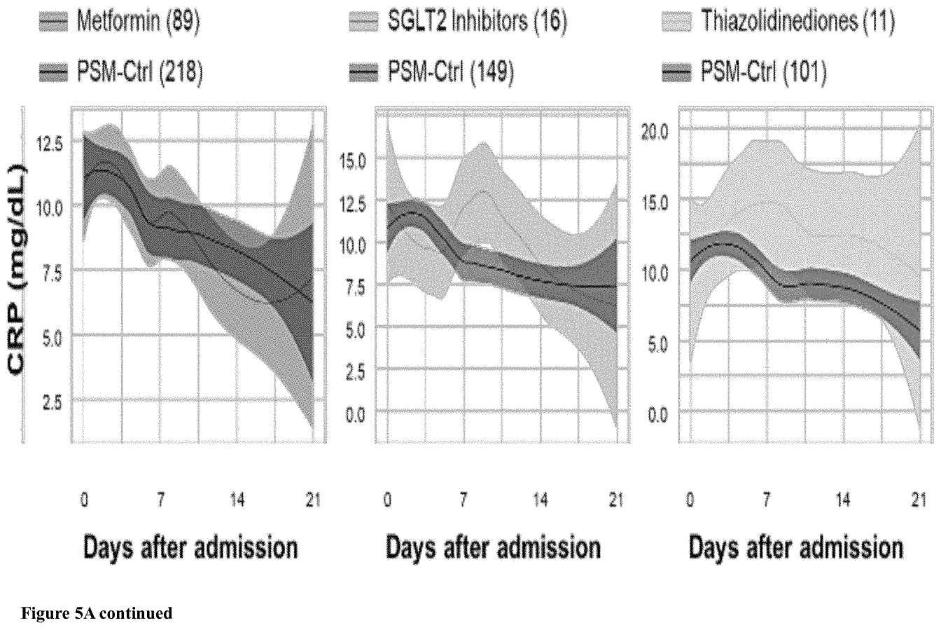

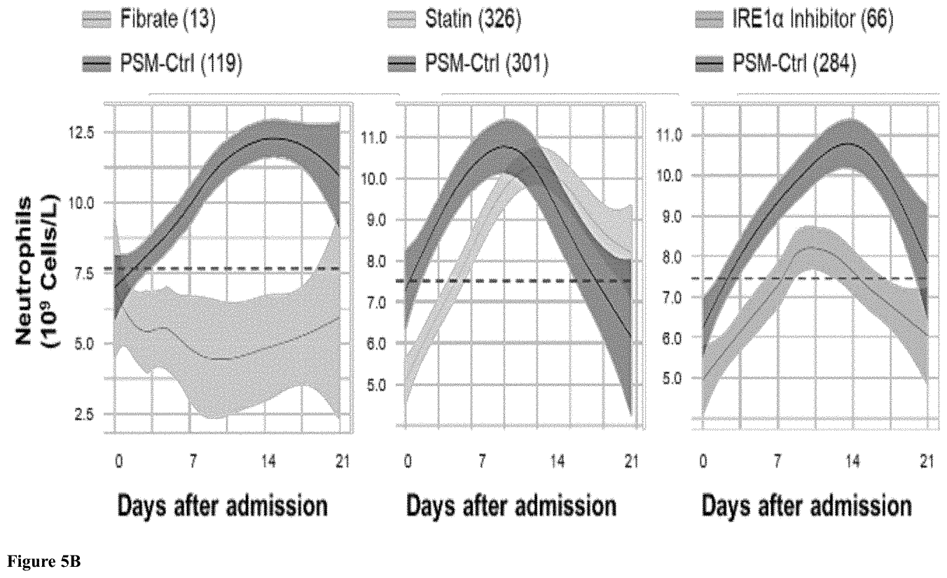

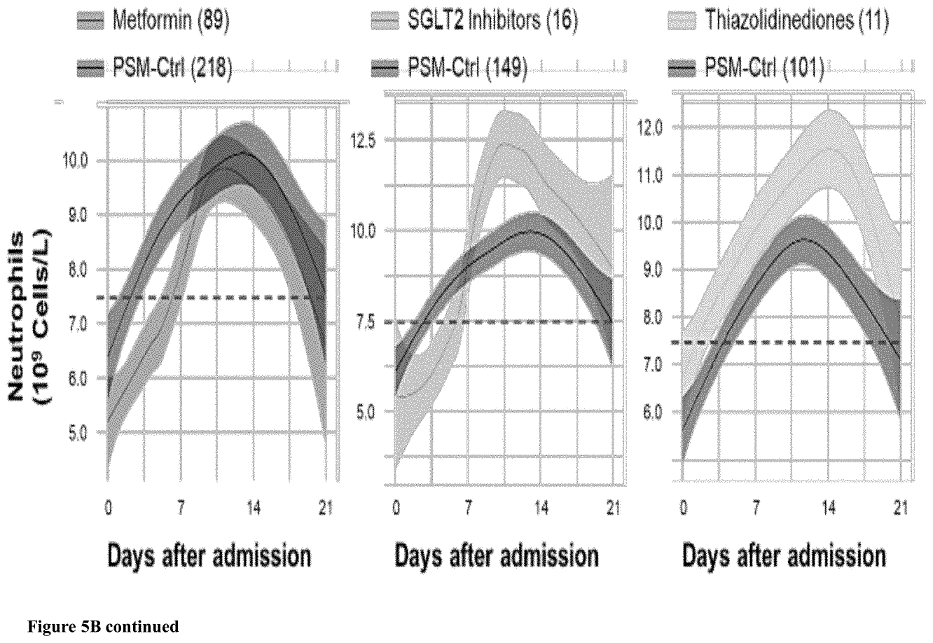

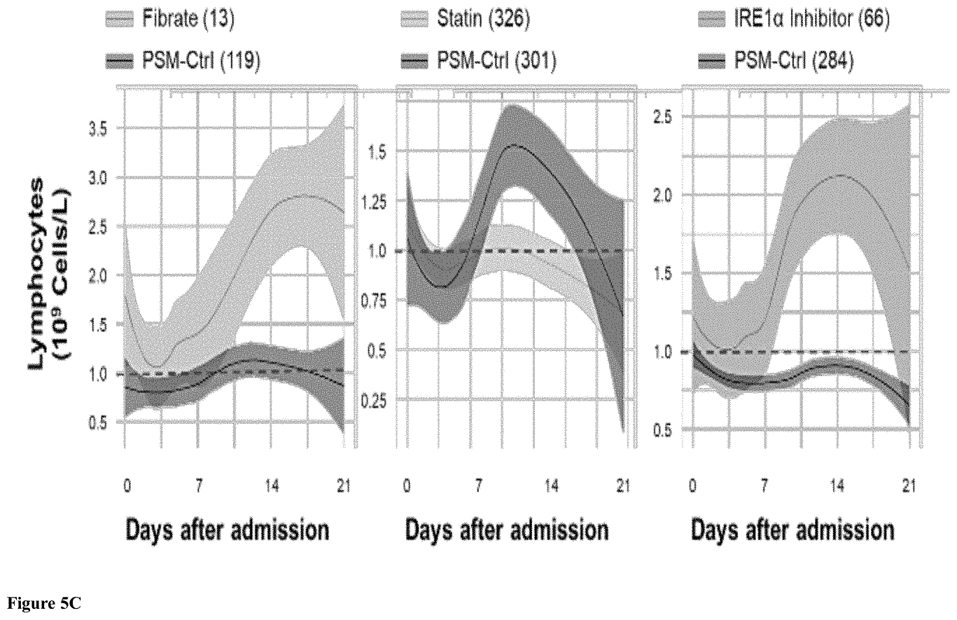

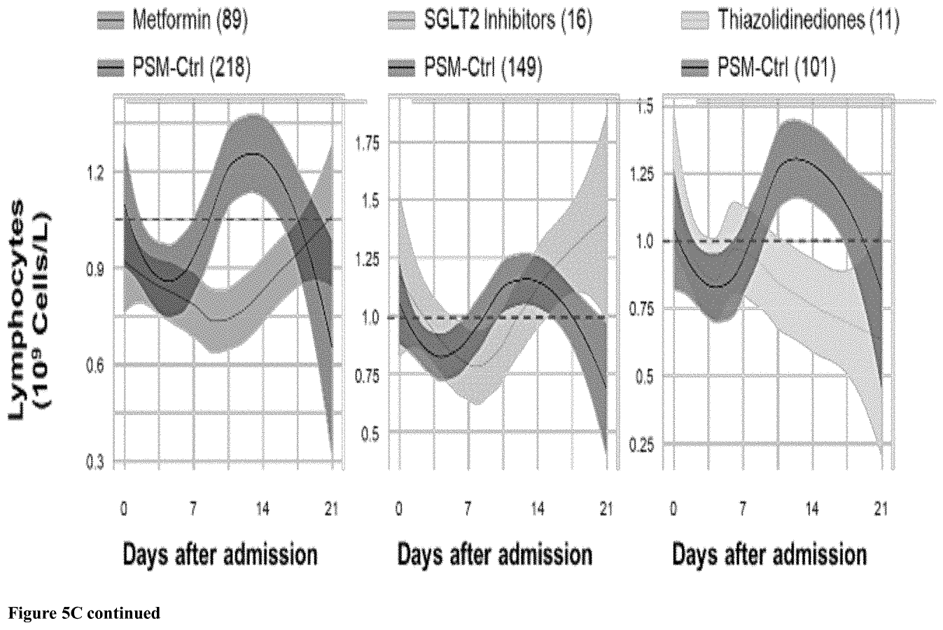

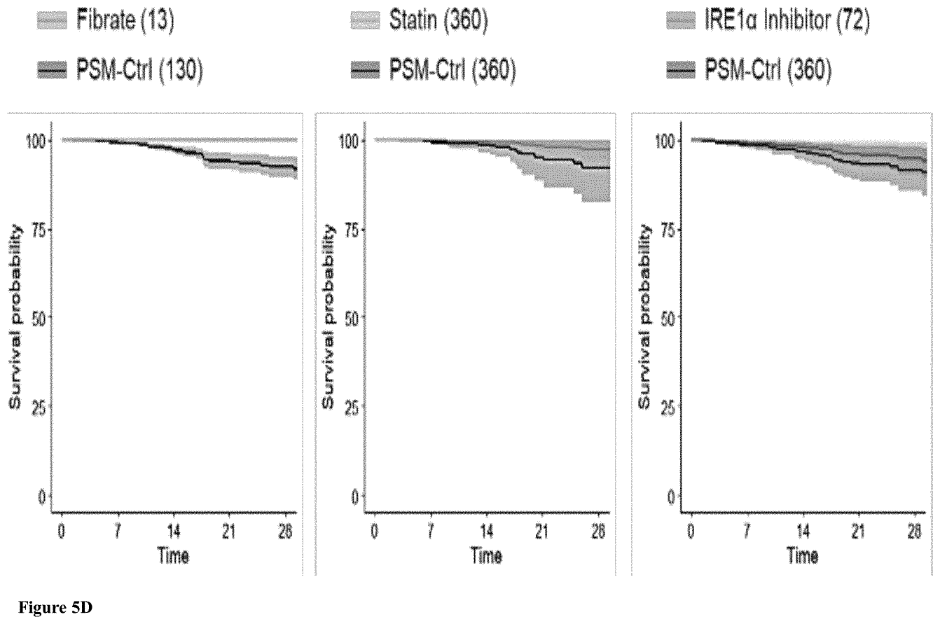

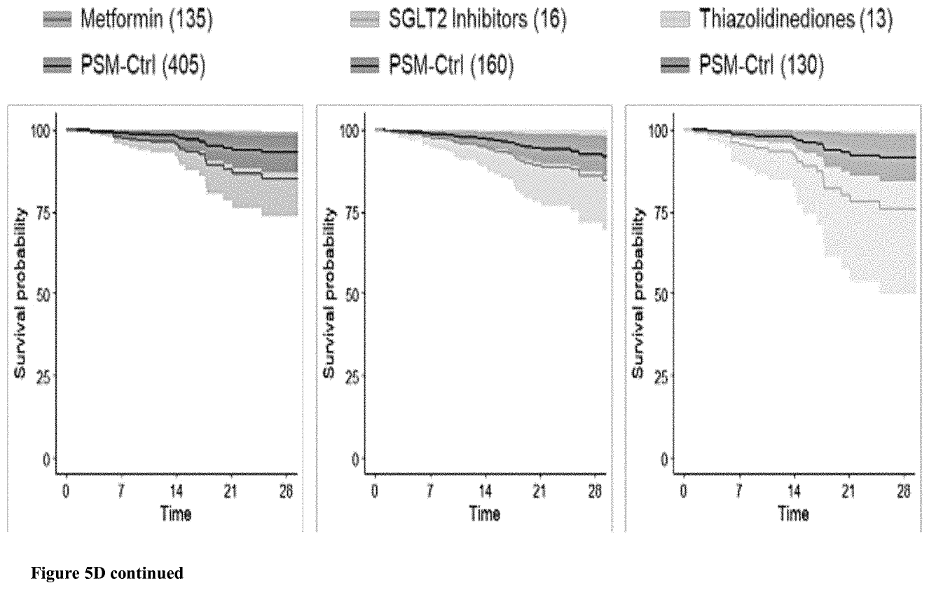

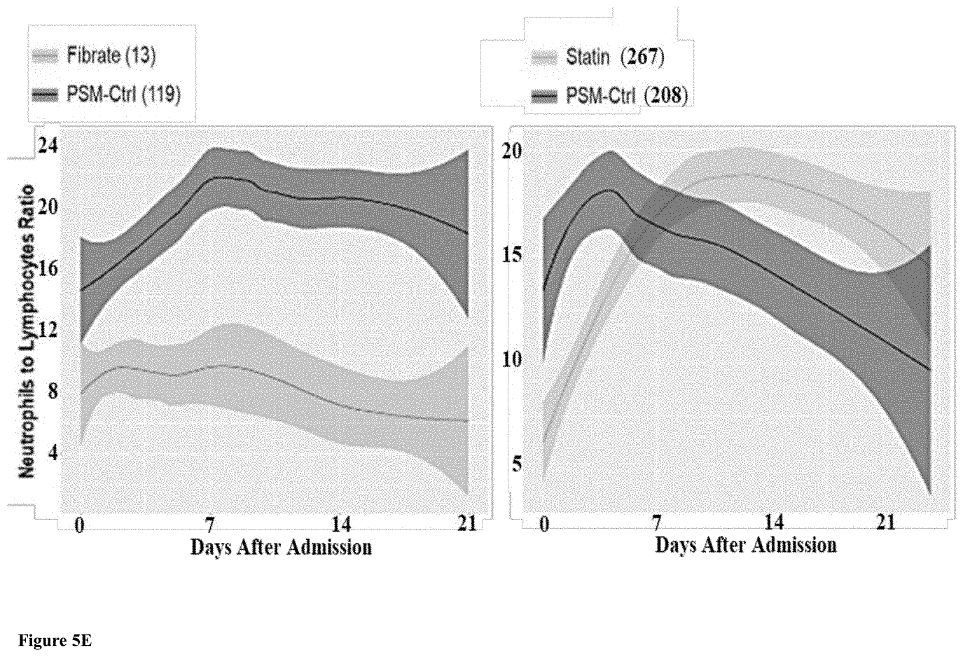

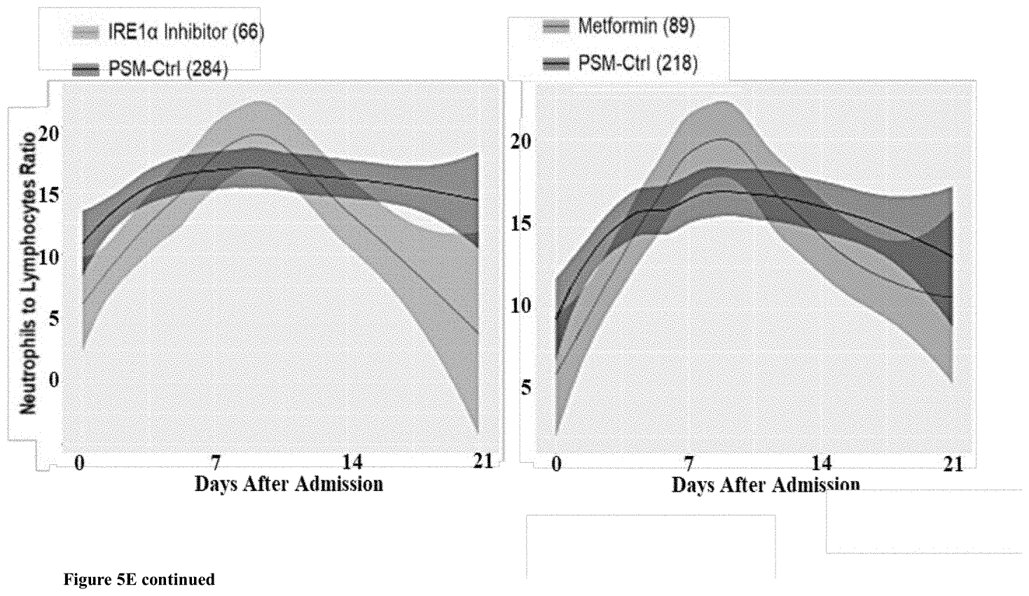

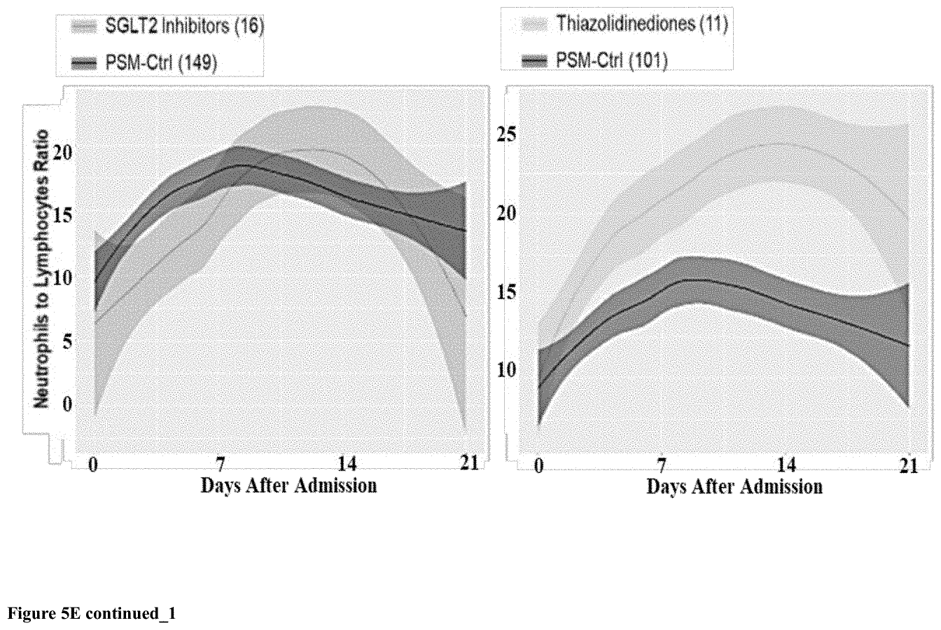

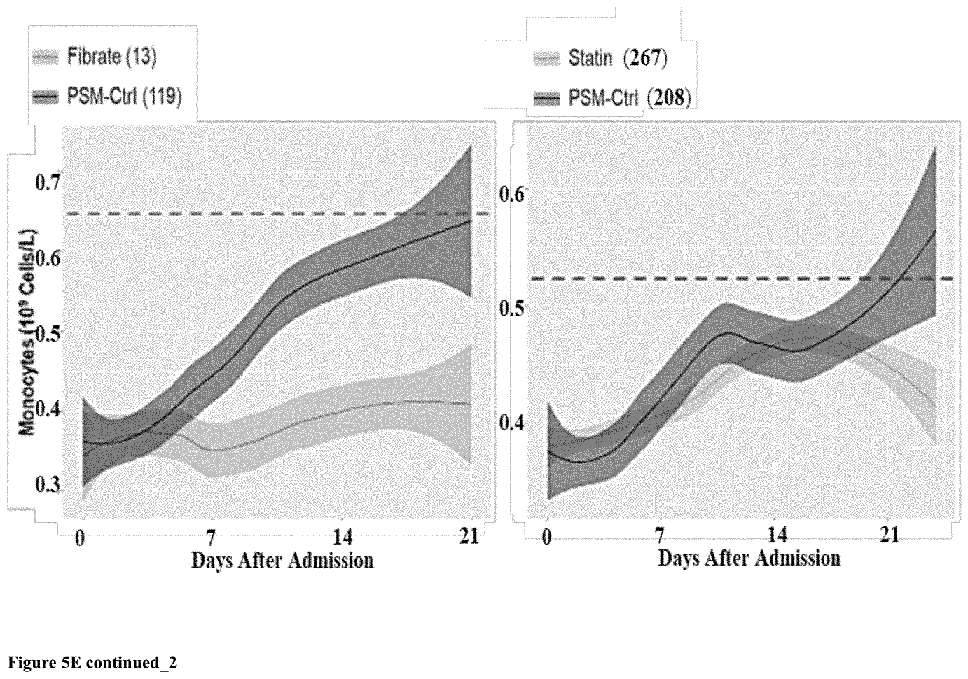

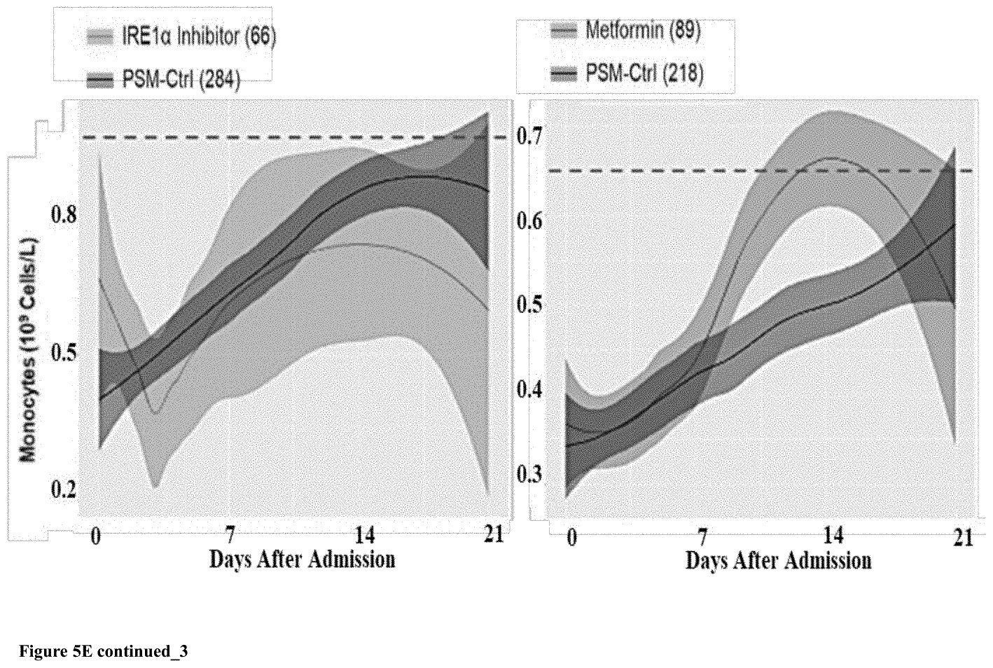

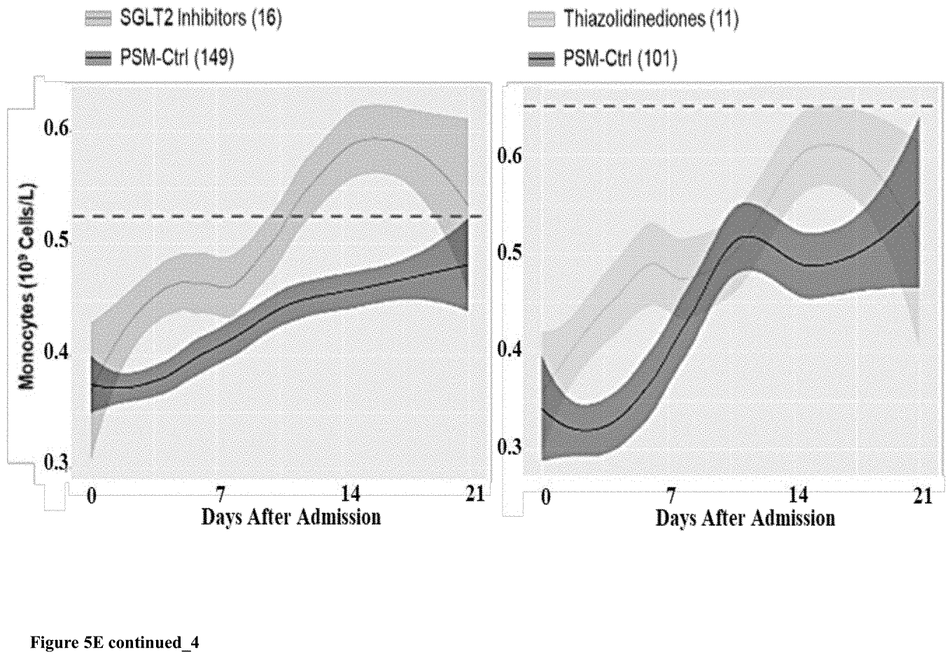

Further, fibrates blocked viral replication over a 5-day period, reversing both lipid accumulation and increased glycolysis at Cmax concentration (FIG. 4A-J). Observational study in Israel confirmed these results showing COVID-19 patients taking bezafibrate or ciprofibrate were underrepresented in ICU admissions and deaths (FIG. 4A-J). Compared to PSM-matched patients, those taking fibrates showed minimal inflammatory response, lower mortality and improved secondary outcomes (FIG. 5A-F, Table 3 and 5).

By a first aspect, there is provided a method of treating coronavirus infection in a subject in need thereof, the method comprising administering a metabolic regulatory drug to the subject, thereby treating coronavirus infection.

By another aspect, there is provided a method of reducing the risk of a symptomatic coronavirus infection in a subject in need thereof, the method comprising administering a metabolic regulatory drug to the subject, thereby reducing the risk of a symptomatic coronavirus infection.

By another aspect, there is provided a metabolic regulatory drug for use in treating a coronavirus infection in a subject in need thereof.

By another aspect, there is provided a metabolic regulatory drug for use in reducing the risk of a symptomatic coronavirus infection in a subject in need thereof.

In some embodiments, the coronavirus is an RNA virus. In some embodiments, the coronavirus infects mammals. In some embodiments, the coronavirus infects humans. In some embodiments, the coronavirus is an Alphacoronavirus. In some embodiments, the coronavirus is an Gammacoronavirus. In some embodiments, the coronavirus is an Deltacoronavirus. In some embodiments, the coronavirus is coronavirus 229E. In some embodiments, the coronavirus is human coronavirus NL63. In some embodiments, the coronavirus is human coronavirus is OC43. In some embodiments, the coronavirus is human coronavirus HKU1. In some embodiments, the corona virus is from the genus Betacoronavirus. In some embodiments, the corona virus is from the subgenus Sarbecovirus. In some embodiments, the coronavirus is Severe Acute Respiratory Syndrome (SARS)-CoV-1. In some embodiments, the coronavirus is SARS-CoV-2. In some embodiments, the coronavirus is Middle East Respiratory Syndrome (MERS). In some embodiments, the coronavirus is selected from SARS-CoV-1, SARS-CoV-2 and MERS. In some embodiments, the coronavirus is a betacoronavirus selected from human coronavirus OC43, human coronavirus HKU1, SARS-CoV-1, SARS-CoV-2 and MERS. In some embodiments, a coronavirus infection results in coronavirus disease. In some embodiments, the disease is SARS. In some embodiments, SARS-CoV-1 infection causes SARS. In some embodiments, the disease is MERS. In some embodiments, MERS infection causes MERS. In some embodiments, the disease is COVID-19. In some embodiments, the SARS-CoV-2 infection causes COVID-19.

Severe acute respiratory syndrome coronavirus 2 (SARS-CoV-2), formerly known as the 2019 novel coronavirus (2019-nCoV), is a positive-sense single-stranded RNA virus. It is contagious among humans and is the cause of coronavirus disease 2019 (COVID-19). SARS-CoV-2 has strong genetic similarity to known bat coronaviruses, making a zoonotic origin in bats likely, although an intermediate reservoir such as a pangolin is thought to be involved. From a taxonomic perspective SARS-CoV-2 is classified as a strain of the species severe acute respiratory syndrome-related coronavirus. SARS-CoV-2 is the cause of the ongoing 2019-20 coronavirus outbreak, a Public Health Emergency of International Concern that originated in Wuhan, China. Because of this connection, the virus is sometimes referred to informally, among other nicknames, as the “Wuhan coronavirus”. In some embodiments, the SARS-CoV-2 is the original SARS-CoV-2. In some embodiments, the original SARS-CoV-2 is the SARS-CoV-2 discovered in Wuhan. In some embodiments, the original SARS-CoV-2 is a non-variant SARS-CoV-2. In some embodiments, the SARS-CoV-2 is a variant of SARS-CoV-2. SARS-CoV-2 variants are well known in the art and include for example the British variant, the South African variant, and the Brazilian variant. It will be understood by a skilled artisan that all SARS-CoV-2 infections result in abnormal lipid deposition and thus all forms of SARS-CoV-2 infection may be treated by a method of the invention.

In some embodiments, the subject is a subject susceptible to coronavirus infection. In some embodiments, the subject is avian. In some embodiments, the subject is a mammal. In some embodiments, the subject is feline. In some embodiments, the subject is a primate. In some embodiments, the subject is a human.

In some embodiments, the subject is infected by a coronavirus. In some embodiments, the subject has a confirmed coronavirus infection. Coronavirus infection can be confirmed by any method known in the art. Commonly diagnosis is performed by a PCR test. Methods for performing PCR testing are known in the art and can be found for example in WHO interim guidance 19 Mar. 2020: Laboratory testing for coronavirus disease (COVID-19) in suspected human cases (apps.who.int/iris/rest/bitstreams/1271387/retrieve), herein incorporated by reference in its entirety.

In some embodiments, the subject was infected at most 1, 2, 3, 4, 5, 6, 7, 8, 9, or 10 days before the administering. Each possibility represents a separate embodiment of the invention. In some embodiments, the administering is immediately after diagnosis of the infection. In some embodiments, immediately is within 1, 2, 4, 5, 6, 8, 12, 18, 24, 36, 48, 72, 96, 120, 148, or 172 hours. Each possibility represents a separate embodiment of the invention. In some embodiments, immediately is within 24 hours. In some embodiments, immediately is within 48 hours.

Each possibility represents a separate embodiment of the invention. In some embodiments, infection is symptoms onset. In some embodiments, infection is diagnosis. In some embodiments, infection is estimated to be 5 days before symptoms onset. In some embodiments, the subject was infected at most 7 days before the administering. In some embodiments, the subject was infected at most 5 days before the administering. In some embodiments, the subject was infected at most 4 days before the administering. In some embodiments, the subject was infected 3-7 days before the administering. In some embodiments, the subject was infected 3-6 days before the administering. In some embodiments, the subject was infected 3-5 days before the administering. In some embodiments, the subject was infected 3-4 days before the administering. In some embodiments, the subject was infected 4-7 days before the administering. In some embodiments, the subject was infected 4-6 days before the administering. In some embodiments, the subject was infected 4-5 days before the administering. In some embodiments, the subject was infected 5-7 days before the administering. In some embodiments, the subject was infected 5-6 days before the administering. In some embodiments, the subject was infected 6-7 days before the administering.

In some embodiments, the subject is in the first phase of coronavirus infection. In some embodiments, the first phase is the first stage. In some embodiments, the first phase is early infection. In some embodiments, the first phase is pre-symptomatic. In some embodiments, the first phase comprises upper respiratory tract infection. In some embodiments, the first phase comprises upper respiratory tract symptoms. In some embodiments, the subject is in the second phase of coronavirus infection. In some embodiments, the second phase is the second stage. In some embodiments, the second phase is the pulmonary phase. In some embodiments, the second stage comprises two parts IIA and IIB. In some embodiments, stage IIA comprises pneumonia without hypoxia. In some embodiments, stage IIB comprises pneumonia with hypoxia. In some embodiments, the second phase comprises lower respiratory tract infection. In some embodiments, the second stage comprises pneumonia. In some embodiments, the subject is in either phase 1 or phase 2.

In some embodiments, the subject is not in phase 3. In some embodiments, phase 3 is stage 3. In some embodiments, phase 3 is the hyperinflammation phase. In some embodiments, phase 3 comprises cytokine storm. In some embodiments, phase 3 comprises ARDS. In some embodiments, phase 3 comprises ICU entrance. In some embodiments, phase 3 comprises mechanical ventilation. In some embodiments, phase 3 is the acute phase of infection. In some embodiments, the subject has not yet entered an acute phase of infection.

In some embodiments, the subject suffers from subclinical inflammation. In some embodiments, subclinical inflammation is an inflammation score from 6 to 9. In some embodiments, the subject suffers from moderate inflammation. In some embodiments, moderate inflammation is a score from 9 to 18. In some embodiments, the subject suffers from severe inflammation. In some embodiments, the subject does not suffer from severe inflammation. In some embodiments, severe inflammation is a score from 18 and above. In some embodiments, the score is a chest CT severity score of lung inflammation. In some embodiments, the score is the Neutrophil to lymphocyte ratio in the subject. In some embodiments, the subject display lung lesions on a chest CT. In some embodiments, the subjects do not display lung lesions on a chest CT. In some embodiments, the subject cannot hold oxygen saturation of above 93%. In some embodiments, the subject can hold oxygen saturation above 93% unaided. In some embodiments, the subject cannot hold oxygen saturation of above 95%. In some embodiments, the subject can hold oxygen saturation above 95% unaided.

In some embodiments, the subject is at risk of a coronavirus infection. In some embodiments, a subject at risk is a non-vaccinated subject. In some embodiments, a subject at risk is a subject with an immunodeficiency. In some embodiments, a subject at risk is a subject with at least one comorbidity. In some embodiments, a subject at risk is a subject in a region/location with a high infection rate. In some embodiments, a high infection rate is a rate of infection above 1, 2, 3, 4, 5, 6, 7, 8, 9, 10, 11, 12, 13, 14, 15, 16, 17, 18, 19 or 20% infection. Each possibility represents a separate embodiment of the invention. In some embodiments, a high infection rate is a rate of infection above 10% infection. In some embodiments, a subject at risk is a front-line worker. In some embodiments, a subject at risk is a medical worker. In some embodiments, a subject at risk is a nursing home worker. In some embodiments, a subject at risk is a subject who cannot be vaccinated. In some embodiments, a subject at risk is anyone during a pandemic.

In some embodiments, the subject does not suffer from a metabolic disease or disorder. As used herein, the terms “metabolic disease” and “metabolic disorder” are synonymous and refer to a condition in which normal metabolism is disrupted. In some embodiments, a metabolic disease is an energy homeostasis disease. In some embodiments, the metabolic disease is a metabolic disease treatable by a metabolic regulatory drug. In some embodiments, the metabolic disease is a metabolic disease treatable by PPARA agonists. In some embodiments, the metabolic disease is a metabolic disease treatable by IRE1 pathway inhibitors. In some embodiments, the subject does not suffer from a disease treatable by a metabolic regulatory drug. In some embodiments, the subject does not suffer from a disease treatable by a PPARA agonist. In some embodiments, the subject does not suffer from a disease treatable by a IRE1 pathway inhibitor. In some embodiments, the subject does not suffer from a metabolic disease treatable by fibrates. In some embodiments, a metabolic disease treatable by a IRE1 pathway inhibitor is hypertension. In some embodiments, a metabolic disease treatable by a PPARA agonist is dyslipidemia. In some embodiments, a metabolic disease treatable by fibrates is dyslipidemia. In some embodiments, dyslipidemia comprises hyperglyceridemia. In some embodiments, dyslipidemia is hyperglyceridemia. In some embodiments, hyperglyceridemia is hypertriglyceridemia. In some embodiments, the subject is not currently being treated by a metabolic regulatory drug. In some embodiments, the subject is not currently being treated by a PPARA agonist. In some embodiments, the subject is not currently being treated by a IRE1 pathway inhibitor.

In some embodiments, the metabolic disease is obesity. In some embodiments, the metabolic disease is metabolic syndrome. In some embodiments, the metabolic disease is diabetes mellitus. In some embodiments, the metabolic disease is dyslipidemia. In some embodiments, the metabolic disease is coronary heart disease. In some embodiments, the metabolic disease is hypertension. In some embodiments, the metabolic disease is hyperglyceridemia. In some embodiments, the metabolic disease is hypertriglyceridemia.

In some embodiments, the subject does not suffer from a comorbidity. In some embodiments, the subject does not suffer from a metabolic comorbidity. In some embodiments, the subject does not suffer from a comorbidity treatable by a metabolic regulatory drug. In some embodiments, the subject does not suffer from a comorbidity treatable by a PPARA agonist. In some embodiments, the subject does not suffer from a comorbidity treatable by a IRE1 pathway inhibitor. In some embodiments, the subject is not currently being treated by a metabolic regulatory drug. In some embodiments, the subject has not previously been treated with a metabolic regulatory drug. In some embodiments, the subject is not currently being treated by a PPARA agonist. In some embodiments, the subject is not currently being treated by a IRE1 pathway inhibitor. In some embodiments, the subject has not previously been treated by a PPARA agonist. In some embodiments, the subject has not previously been treated by a IRE1 pathway inhibitor. In some embodiments, a subject being treated with a first metabolic regulatory drug for a condition that is not viral infection is treated with a second metabolic regulatory drug. In some embodiments, a subject already being treated by a PPARA agonist is treated with a IRE1 pathway inhibitor. In some embodiments, a subject already being treated by a IRE1 pathway inhibitor is treated with a PPARA agonist.

In some embodiments, the subject suffers from at least one comorbidity. In some embodiments, comorbidity is comorbidity with the coronavirus. In some embodiments, the comorbidity is selected from hypertension, diabetes mellitus, coronary heart disease, cerebrovascular diseases, obesity, dyslipidemia, asthma, chronic obstructive pulmonary disease (COPD), chromic liver disease, and chronic kidney diseases. In some embodiments, the comorbidity is hypertension. In some embodiments, the comorbidity is diabetes mellitus. In some embodiments, the comorbidity is coronary heart disease. In some embodiments, the comorbidity is cerebrovascular disease. In some embodiments, the comorbidity is obesity. In some embodiments, the comorbidity is dyslipidemia. In some embodiments, the comorbidity is asthma. In some embodiments, the comorbidity is COPD. In some embodiments, the comorbidity is chromic liver disease. In some embodiments, the comorbidity is chronic kidney disease.

In some embodiments, a metabolic regulatory drug is administered. In some embodiments, a PPARA agonist is administered. In some embodiments, a IRE1 pathway antagonist is administered. In some embodiments, a composition is administered. In some embodiments, the composition is a pharmaceutical composition. In some embodiments, the composition is a therapeutic composition. In some embodiments, the composition comprises a metabolic regulatory drug. In some embodiments, the composition comprises the PPARA agonist. In some embodiments, the composition comprises the IRE1 pathway antagonist. In some embodiments, the composition comprises a pharmaceutically acceptable carrier, excipient or adjuvant. In some embodiments, the composition is formulated for oral administration. In some embodiments, the composition is formulated for systemic administration. In some embodiments, the composition is formulated for intravenous administration.

In some embodiments, a metabolic regulatory drug is a metabolic regulator. In some embodiments, a metabolic regulatory drug is selected from a PPARA agonist and a IRE1 pathway inhibitor. In some embodiments, a metabolic regulatory drug is selected from a statin, a PPARA agonist and a IRE1 pathway inhibitor. In some embodiments, a metabolic regulatory drug is selected from a glycolysis inhibitor, a PPARA agonist and a IRE1 pathway inhibitor. In some embodiments, a metabolic regulatory drug is selected from a statin, a glycolysis inhibitor, a PPARA agonist and a IRE1 pathway inhibitor.

In some embodiments, the metabolic regulatory drug is a PPARA agonist. In some embodiments, the PPARA agonist is a PPARA specific agonist. In some embodiments, the PPARA agonist does not agonize PPAR gamma (PPARG). In some embodiments, the PPARA agonist does not significantly agonize PPARG. In some embodiments, the PPARA agonist produces a greater agonizing effect on PPARA than on PPARG. In some embodiments, the greater effect is at least a 2-fold, 3-fold, 4-fold, 5-fold, 6-fold, 7-fold, 8-fold, 9-fold, or 10-fold greater effect. Each possibility represents a separate embodiment of the invention. In some embodiments, the greater effect is at least a 10-fold greater effect. In some embodiments, the PPARA agonist does agonize PPARG. In some embodiments, the PPARA agonist is a fibrate. In some embodiments, the PPARA agonist is pirinixic acid. In some embodiments, pirinixic acid is WY-14,643. In some embodiments, the PPARA agonist is a conjugated linoleic acid (CLA). In some embodiments, the PPARA agonist is selected from a fibrate, pirinixic acid and a CLA. In some embodiments, the PPARA agonist is selected from a fibrate, pirinixic acid, a CLA, MD001, LY518674, K111, ZYH7, and Macuneos.

CLAs are well known in the art and include at least 28 isomers of linoleic acid. In some embodiments, the CLA is a mix of isomers. In some embodiments, the isomer is 9-CLA. In some embodiments, 9-CLA is rumenic acid. In some embodiments, the isomer is 10-CLA. In some embodiments, the CLA is 9-CLA. In some embodiments, the CLA is 10-CLA. In some embodiments, the CLA is selected from 9-CLA and 10-CLA.

Fibrates are a class of amphipathic carboxylic acids that are well known in the art. In some embodiments, a fibrate treats a metabolic disorder. In some embodiments, a fibrate treats high cholesterol (hypercholesterolemia). In some embodiments, a fibrate treats hyperglyceridemia. In some embodiments, a fibrate treats dyslipidemia. In some embodiments, a fibrate treats hypertriglyceridemia. In some embodiments, treating is the drug's primary treatment. In some embodiments, the fibrate is selected from aluminum clofibrate, bezafibrate, ciprofibrate, choline fenofibrate, clinofibrate, clofibrate, clofibride, fenofibrate, gemfibrozil, ronifibrate and simfibrate. In some embodiments, the fibrate is selected from Fenofibrate (Fenoglide/Tricor), Gemfibrozil (Lopid), Clofibrate (Atromid-S), Clinofibrate (Lipoclin), Bezafibrate (Bezalip), Simfibrate, Ronifibrate, Clofibride, and Ciprofibrate. In some embodiments, the fibrate is fenofibrate. In some embodiments, the fibrate is not gemfibrozil. In some embodiments, the fibrate is selected from fenofibrate, bezafibrate, gemfibrozil, pemafibrate, and ciprofibrate.

In some embodiments, the metabolic regulatory drug is a IRE1 pathway inhibitor. In some embodiments, the IRE1 pathway inhibitor is an inhibitor of IRE1 alpha (IRE1α). In some embodiments, an IRE1 pathway inhibitor inhibits ER stress. In some embodiments, a IRE1 pathway inhibitor treats hypertension. In some embodiments, the IRE1 pathway inhibitor is telmisartan. In some embodiments, the IRE1 pathway inhibitor is selected from telmisartan, Sunitinib, STF-083010, 4 μ8C, KIRA6m, Kira8, Kira7, MKC8866, GSK2850163, Toyocamycin, APY29, MKC3946, MKC9989, NSC95682, B-I09, 3,6-DMAD, and IRE1α kinase-IN-2.

In some embodiments, the metabolic regulatory drug is a statin. Statins are well known in the art and include for example simvastatin (Zocor) and Pravastatin (Pravachol) among many others. In some embodiments, a statin is an HMG-CoA reductase inhibitor. In some embodiments, a statin is a high cholesterol treatment. In some embodiments, a statin is a diabetes mellitus treatment.

In some embodiments, the metabolic regulatory drug is a glycolysis inhibitor. In some embodiments, the glycolysis inhibitor is an inhibitor of at least one of GLUT1, SGLT2 and SGLT1. In some embodiments, the glycolysis inhibitor is an inhibitor of SGLT2. In some embodiments, a glycolysis inhibitor is an inhibitor of glucose transport. Such inhibitors are well known in the art and include, but are not limited to Dapagliflozin (Forxiga), Canagliflozin (Invokana), Empagliflozin (Jardiance), Ertugliflozin (Steglatro), Sotagliflozin (Zynquista), Quinidine, Cloperastine (Hustazol), Bepridil, Trihexyphenidyl, Bupivacaine, (+)-ε-viniferin, (+)-pteryxin, BAY 876, WZB-117, STF-31, and Fasentin.

In some embodiments, the metabolic regulatory drug is an AMPK activator. In some embodiments, the AMPK activator is metformin. AMPK activators are well known in the art and include for example metformin, phenformin (DBI), buformin (Silubin), Proguanil, Chlorproguanil and AICAR.

In some embodiments, a metabolic regulatory drug is not a thiazolidinedione. In some embodiments, a metabolic regulatory drug is not metformin. In some embodiments, a metabolic regulatory drug is not an AMPK activator. In some embodiments, a metabolic regulatory drug is not a statin. In some embodiments, a metabolic regulatory drug is not a glycolysis inhibitor.

As used herein, the term “carrier,” “adjuvant” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life.

In some embodiments, treating comprises treating at least one symptom of a coronavirus. Symptoms of coronavirus are well known in the art, and include, but are not limited to, fever, cough, runny nose, fatigue, muscle aches, sore throat, diarrhea, headache, anosmia, ageusia, skin rash, difficulty breathing, shortness of breath, chest pain, chest pressure, loss of speech, and disorientation. In some embodiments, treating comprises reducing phospholipid accumulation. In some embodiments, the accumulation in within a lung of the subject. In some embodiments, the accumulation in within lung tissue. In some embodiments, the accumulation is within lung cells. In some embodiments, the lung cells are lung epithelial cells. In some embodiments, treating comprises reducing viral load. In some embodiments, viral load is viral load in the subject. In some embodiments, treating comprises reducing symptoms. In some embodiments, treating comprises reducing inflammation. In some embodiments, inflammation is inflammation in the subject. In some embodiments, inflammation is systemic inflammation. In some embodiments, inflammation is lung inflammation. In some embodiments, inflammation is characterized by levels of C-reactive protein (CRP). In some embodiments, treating comprises reducing CRP levels. In some embodiments, treating comprises reducing the risk of entering phase 3 of the disease. In some embodiments, treating comprises reducing a risk of mechanical ventilation. In some embodiments, mechanical ventilation is invasive mechanical ventilation. In some embodiments, treating comprises reducing a risk of septic shock. In some embodiments, treating comprises reducing a risk of acute liver injury. In some embodiments, treating comprises reducing a risk of acute kidney injury. In some embodiments, treating comprises reducing a risk of acute cardiac injury. In some embodiments, treating comprises reducing a risk of ICU admission. In some embodiments, treating comprises reducing a risk of hospitalization. In some embodiments, treating comprises reducing hospitalization time. In some embodiments, treating comprises reducing hospitalization length. In some embodiments, treating comprises reducing a risk of developing Acute respiratory distress syndrome (ARDS). n some embodiments, treating comprises reducing a risk of developing a cytokine storm. In some embodiments, treating comprises reducing risk of death. In some embodiments, treating comprises reducing death.

In some embodiments, treating an infection is treatment post-acute disease. In some embodiments, post-acute disease is post-acute sequalae disease. In some embodiments, the symptoms are symptoms post-acute disease. In some embodiments, treating a coronavirus infection is treating symptoms post-infection. In some embodiments, treating post-acute disease symptoms is treating long COVID. As used herein, the term “long COVID” refers to post-acute sequalae of SARS-CoV-2 infection, also known as post-acute sequelae of COVID-19 (PASC), chronic COVID syndrome (CCS) and long-haul COVID. It refers to a condition characterized by long-term, persistent coronavirus symptoms.

In some embodiments, treating occurs within 10, 9, 8, 7, 6, 5, 4, 3, 2 or 1 day. Each possibility represents a separate embodiment of the invention. In some embodiments, treating occurs within 5 days. In some embodiments, treating occurs in 3-5 days. In some embodiments, treating occurs in 1-7, 1-6, 1-5, 1-4, 1-3, 1-2, 2-7, 2-6, 2-5, 2-4, 2-3, 3-7, 3-6, 3-5, 3-4, 4-7, 4-6, or 4-5 days. Each possibility represents a separate embodiment of the invention. In some embodiments, treating occurring is treating starting. In some embodiments, treating occurring is treating completing. In some embodiments, at least one symptom improves within the above recited time. In some embodiments, the subject is discharged from the hospital within the above recited time. In some embodiments, the subject is cured with the above recited time.

In some embodiments, decreasing the risk of symptomatic infection is causing asymptomatic infection. In some embodiments, decreasing the risk of symptomatic infection results in asymptomatic infection. In some embodiments, decreasing the risk of symptomatic infection is preventing symptomatic infection. In some embodiments, preventing symptomatic infection is in a subject that is not currently infected. In some embodiments, preventing symptomatic infection is a result of prophylactic treatment.

As used herein, the terms “administering,” “administration,” and like terms refer to any method which, in sound medical practice, delivers a composition containing an active agent to a subject in such a manner as to provide a therapeutic effect. One aspect of the present subject matter provides for oral administration of a therapeutically effective amount of a composition of the present subject matter to a patient in need thereof. In some embodiments, the administration is systemic administration. In some embodiments, the administration is oral administration. In some embodiments, the administration is intravenous administration. Other suitable routes of administration can include parenteral, subcutaneous, intravenous, aerosol, or intraperitoneal.

The dosage administered will be dependent upon the age, health, and weight of the recipient, kind of concurrent treatment, if any, frequency of treatment, and the nature of the effect desired. In some embodiments, the dose of the metabolic regulatory drug is the same dose at which it is administered to treat a metabolic condition or disease. In some embodiments, the dose administered to treat a metabolic condition or disease is the standard dose. In some embodiments, the dose of the metabolic regulatory drug is a higher dose than the dose it is administered to treat a metabolic condition or disease. In some embodiments, the dose of the metabolic regulatory drug is the same dose as for its primary treatment. In some embodiments, the dose of the metabolic regulatory drug is higher than for its primary treatment. In some embodiments, a higher dose is a more frequent dose. In some embodiments, a PPARA agonists primary treatment is hypercholesterolemia. In some embodiments, a PPARA agonists primary treatment is hyperglyceridemia. In some embodiments, a PPARA agonists primary treatment is dyslipidemia. In some embodiments, a PPARA agonists primary treatment is hypertriglyceridemia. In some embodiments, a IRE1 pathway inhibitor treats hypertension.

In some embodiments, a higher dose is twice the dose. In some embodiments, twice the does is twice as frequently. In some embodiments, the twice as frequently is twice a day. In some embodiments, twice the dose is a single administration of a dose that is twice the standard dose. In some embodiments, a higher dose is administered on the first day of treatment and the standard dose is administered on subsequent days. In some embodiments, twice the dose is administered on the first day of treatment and the standard dose is administered on subsequence days. It will be understood by a skilled artisan, that in order to reach Cmax as quickly as possible and increased dose can be administered at first and then the standard dose can be used to maintain Cmax in the subject. In some embodiments, the dose is a dose selected from one provided in Table 1. In some embodiments, the standard dose is a dose selected from one provided in Table 1. In some embodiments, the dose of fenofibrate is 40 to 120 mg/day. In some embodiments, the dose of fenofibrate is 40 to 150 mg/day. In some embodiments, the dose of fenofibrate is about 46 mg/day. In some embodiments, the dose of fenofibrate is about 145 mg/day.

TABLE 1
Standard dosing for metabolic drugs
(mg/kg/day is the dose in rodents)

| | |
|---|---|
| Drug | Dose |

Glycolosis Inhibitors

| | | |
|---|---|---|
| Dapagliflozin (Forxiga) | 5 to 10 | mg/day |
| Canagliflozin (Invokana) | 100 to 300 | mg/day |
| Empagliflozin (Jardiance) | 10 to 25 | mg/day |
| Ertugliflozin (Steglatro) | 5 to 15 | mg/day |
| Sotagliflozin (Zynquista) | 200 to 400 | mg/day |
| Quinidine | 200 to 800 | mg/day |
| Cloperastine (Hustazol) | 8 to 60 | mg/day |
| Bepridil | 200 to 400 | mg/day |
| Trihexyphenidyl | 1 to 15 | mg/day |
| Bupivacaine | 0.25% to 0.9% | solution |
| (+)-ε-viniferin | 48 | μM |
| (+)-pteryxin | 12 | μM |
| BAY 876 | 1.5-4.5 | mg/kg/day |
| WZB-117 | 10-30 | mg/kg/day |
| STF-31 | 4-30 | mg/kg/day |
| Fasentin | 5-60 | mg/kg/day |

PPARA Agonists

| Fenofibrate nanonised (Tricor-3) | 50 to 145 | mg/day |
|---|---|---|
| Fenofibrate micronized (Tricor-2) | 60 to 160 | mg/day |
| Fenofibrate (Tricor-1) | 60 to 200 | mg/day |
| Fenofibric acid (Trilipix) | 45 to 135 | mg/day |
| Gemfibrozil (Lopid) | 600 mg/12 hrs | |
| Clofibrate (Atromid-S) | 500 mg/6 hrs | |
| Clinofibrate (Lipoclin) | 400 to 800 | mg/day |
| Bezafibrate (Bezalip) | 400 | mg/day |
| 9CLA | 1000 | mg/day |

AMPK Activators

| Metformin | 500 mg twice a day or | |
|---|---|---|
| | 850 mg once a day | |
| Phenformin (DBI) | 100 to 800 | mg/day |
| Buformin (Silubin) | 150 to 800 | mg/day |
| Proguanil | 400 to 1000 | mg/day |
| Chlorproguanil | 15 to 240 | mg/day |
| AICAR | 20-200 | mg/kg/day |

Statins

| | | |
|---|---|---|
| Pravastatin (Pravachol) | 10 to 80 | mg/day |
| Simvastatin (Zocor) | 5 to 80 | mg/day |

In some embodiments, the metabolic regulatory drug is formulated to reach a Cmax in said subject within 1 day from administration. In some embodiments, the metabolic regulatory drug is formulated to reach a Cmax in said subject within 2 days from administration. In some embodiments, the metabolic regulatory drug is formulated to reach a Cmax in said subject within 3 day from administration. In some embodiments, metabolic regulatory drug is formulated to reach a Cmax in said subject rapidly. In some embodiments, the metabolic regulatory drug is administered in order to reach a Cmax in the subject rapidly. In some embodiments, rapidly is within 12 hours, 18 hours, 24 hours, 2 days, 3 days, 4 days or 5 days. Each possibility represents a separate embodiment of the invention. In some embodiments, rapidly is within 1 day. In some embodiments, rapidly is within 3 days. In some embodiments, rapidly is within 5 days. In some embodiments, rapidly is before onset of severe disease. In some embodiments, severe disease is acute disease. In some embodiments, acute disease is severe disease. In some embodiments, severe disease comprises cytokine storm. In some embodiments, severe disease comprises ARDS. In some embodiments, severe disease comprises mechanical ventilation.

It will be understood by a skilled artisan that the therapeutic agent needs to take effect before the subject reaches a point at which the therapy is no longer effective. However, the window between diagnosis and severe disease is often very short, as such, Cmax must be reached rapidly. Certain formulations are known to result in higher bioavailability and thus in a Cmax reached more rapidly. For example, it is known that many fibrates take a long time to reach Cmax, however, certain fibrate formulations (e.g., nanocrystal formulations) are known to reach Cmax more rapidly (e.g., within a day). In some embodiments, the formulation is a nanocrystal formulation. In some embodiments, the fibrate is a fibrate nanocrystal. In some embodiments, the fibrate is a fenofibrate nanocrystal. Fenofibrate nanocrystals are known in the art and include for example Tricor® and Triglide®. In some embodiments, the fenofibrate is selected from Tricor® and Triglide®. Intravenous administration is the most rapid way to reach Cmax. In some embodiments, the administration is intravenous administration. In some embodiments, the formulation is an intravenous formulation. Fibrates are generally administered orally, however, intravenous formulations are known in the art. Oral formulations of fibrates are well known in the art and are provided, for example in Ling et al., “A review of currently available fenofibrate and fenofibric acid formulations” 2013, Cardiol. Res.; 4(2):47-55, herein incorporated by reference in its entirety. An intravenous fenofibric acid formulation is disclosed for example in Zhu et al., “Comparison of the gastrointestinal absorption and bioavailability of fenofibrate and fenofibric acid in humans”, 2010, Journal of Clinical Pharmacology, 50:914-921, herein incorporated by reference in its entirety.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

Cell Culture: Normal human bronchial epithelial (NHBE) cells (Lonza, CC-2540 Lot #580580), isolated from a 79-year-old Caucasian female were maintained at 37° C. and 5% CO₂ in bronchial epithelial growth media (Lonza, CC-3171) supplemented with SingleQuots (Lonza, CC-4175) per manufacturer's instructions. NHBE cells (ATCC, PCS-300-010 Lot #63979089; #70002486), isolated from a 69-year-old Caucasian male and a 14-year-old Hispanic male were maintained in airway epithelial cell basal media (ATCC, PCS-300-030) supplemented with Bronchial Epithelial Growth Kit as per the manufacturer's instructions (ATCC, PCS-300-040) at 37° C. and 5% CO₂.

Viruses: SARS-related coronavirus 2 (SARS-CoV-2), Isolate USA-WA1/2020 (NR-52281) was deposited by the Center for Disease Control and Prevention and obtained through BEI Resources, NIAID, NIH. SARS-CoV-2 was propagated in Vero E6 cells in DMEM supplemented with 2% Fetal Bovine Serum (FBS), 4.5 g/L D-glucose, 4 mM L-glutamine, 10 mM Non-Essential Amino Acids (NEAA), 1 mM Sodium Pyruvate, and 10 mM HEPES. Infectious titers of SARS-CoV-2 were determined by plaque assay in Vero E6 cells in Minimum Essential Media (MEM) supplemented with 4 mM L-glutamine, 0.2% Bovine Serum Albumin (BSA), 10 mM HEPES and 0.12% NaHCO₃ and 0.7% agar.

COVID-19 Biopsy Samples: Two COVID19 human subjects were deceased upon tissue acquisition and were provided from Weill Cornell Medicine as fixed samples. Uninfected human lung samples (n=2) were obtained post-surgery through the Mount Sinai Institutional Biorepository and Molecular Pathology Shared Resource Facility (SRF) in the Department of Pathology.

Bioinformatic Analyses: Raw reads were aligned to the human genome (hg19) using the RNA-Seq Alignment App on Basespace (Illumina, CA), following differential expression analysis using DESeq249. Differentially expressed genes (DEGs) were characterized for each sample (p adjusted-value <0.05) and were used as a query to search for enriched biological processes (Gene ontology BP) and network analysis of protein interactions using STRING14.

Analysis of Canonical Splice Variants: Reads were downloaded from SRA (GSE147507), and filtered and trimmed to remove low-quality reads and sequencing artifacts with fastp v20 50 (github.com/OpenGene/fastp.git). Reads were pseudoaligned to the GRCh38 genecode human transcriptome (GRCh38.p13, version 32) using Kallisto version 0.46.1 (github.com/pachterlab/kallisto) run with the default k-mer length of 31, in single-read, single-overhang mode, with fragment mean length of 400 and 100 SD. Differentially expressed transcripts/genes were identified using Sleuth based on a likelihood ratio test comparing the condition of interest and 100 Kallisto bootstrap samples.

Processing, Analysis, and Graphic Display of Genomic Data: Hierarchical clustering, heat maps, correlation plots, and similarity matrices were created in Morpheus. Gene ontology enrichment analyses and clustering were performed using DAVID Informatics Resources 6.7 and PANTHER Classification System. Metabolic network maps were created using McGill's Network Analyst Tool using the KEGG database. Transcription factor networks were created using GeneMania.

Quantification of Intracellular Glucose: To detect glucose uptake, 2-(N-(7-Nitrobenz-2-oxa-1,3-diazol-4-yl) Amino)-2-Deoxyglucose (2-NDBG) a fluorescent analog of glucose (Invitrogen, USA; N13195) was used. 2-NDBG is transported through SGLT-1 and GLUT-2. Increased uptake leads to 2-NDBG accumulation in the cells. Cells infected with SARS-CoV-2 for 96 hours were exposed to 6 mM of 2-NDBG for 24 hours. Cells were then fixed, counterstained with 1 μg/mL Hoechst 33258. Staining intensity was normalized to Hoechst 33258 across multiple fields of view.

Quantification of Lipids: Lipid accumulation was measured using HCS LipidTOX™ Phospholipidosis and Steatosis Detection Kit according to the manufacturer's instructions (ThermoFisher, USA; H34158). Briefly, cells were incubated in complete bronchial epithelial growth media supplemented with 1× phospholipidosis detection reagent for 48 hours. Cells were subsequently fixed in 4% PFA and stained with 1× neutral lipid detected reagent for 30 min and counterstained with 1 μg mL-1 Hoechst 33258. Staining intensity was normalized to the amount of Hoechst 33258 positive nuclei across multiple fields of view.

Metabolic of Analysis Glucose, Lactate, and Glutamine: For metabolic analysis of SARS-CoV-2 infected culture media in the BSL3 facility, the Accutrend Plus multiparameter meter (Roche Diagnostics) was used. Culture media was collected every 48 hours and stored at −80° C. prior to analysis. Measurements were carried out using Accutrend Plus Glucose and BM-Lactate Test Strips according to the manufacturer's instructions. Metabolic analysis of cells cultured in BSL2 facility used the more complete amperometric glucose, lactate, and glutamine sensor array (IST, Switzerland). Measurements were carried out on 3 biological repeats using 2 technical repeats, calibrated throughout the measurement using a calibration media. In both measurements, glucose and glutamine uptake, as well as lactate production, were calculated based on the difference between sample and control media.

Pre-processing and data analysis: The raw 3′ scRNA-seq data were processed using CellRanger version 3.1.0 (10×Genomics). The transcripts were aligned to a customized reference genome in which the SARS-CoV-2 genome (Refseq-ID: NC 045512) was added as an additional chromosome to the human reference genome hg19 (10× Genomics, version 3.0.0). An entry summarizing the entire SARS-CoV-2 genome as one ‘gene’ was appended to the hg19 annotation gtf file, and the genome was indexed using ‘cellranger_mkref’.

After pre-processing, contaminating ambient RNA reads were filtered by using SoupX56 version 1.2.2 (github.com/constantAmateur/SoupX) and MUC1, MUC5AC and MUC5B as marker genes. The filtered expression matrices were loaded into R version 3.6.1 with Seurat version 3.1.4.9012 (github.com/satijalab/seurat), where further filtering was done to remove cells with fewer than 200 genes expressed or more than 15% mitochondrial transcripts. The quality of the scRNA-seq data set was assessed by plotting the number of unique molecular identifiers (UMIs) and genes per cell for each sample. The quality was consistent across samples, and differences in RNA and gene content could be ascribed to cell-type-specific effects.

An upper cutoff for the number of UMIs was manually determined for each sample based on a plot of gene count versus UMI count, with values ranging between 50,000 and 200,000 UMIs. After quality control filtering, the samples were normalized to 10,000 reads, scaled and centered. For integration, 3,000 shared highly variable genes were identified using Seurat's ‘SelectIntegrationFeatures( )’ function. Integration anchors were identified based on these genes using canonical correlation analysis57 with 90 dimensions as implemented in the ‘FindTransferAnchors( )’ function. The data were then integrated using ‘IntegrateData( )’ and scaled again using ‘ScaleData( )’. Principal component analysis (PCA) and uniform manifold approximation and projection (UMAP) dimension reduction with 90 principal components were performed. A nearest-neighbor graph using the 90 dimensions of the PCA reduction was calculated using ‘FindNeighbors( )’, followed by clustering using ‘FindClusters( )’ with a resolution of 0.6.

For comparisons between expression values, the Seurat function ‘FindMarkers( )’ was used with the ‘negbinom’ method and days after onset of symptoms as the confounder variable. Cell type markers were obtained using the ‘FindAllMarkers( )’ function with a negative binomial test and the case severity as the latent variable. Cell type numbers were compared by logistic regression followed by Tukey's test in multivariate cases and otherwise by Fisher's exact test. Weights corresponding to the cell count per sample were introduced into the logistic regression to account for differences in information content. Correlation was assessed using Spearman's P, and significance of correlation was calculated using the corr.test function in R. All tests were two sided. P values were corrected using the Benjamini-Hochberg (false discovery rate) method. Homoskedasticity was assessed using Bartlett's test. Cells were defined as positive for a gene if at least one UMI of that gene was found. Note that sample numbers in the figure legends refer to biological samples, and the n for statistical calculations is derived from the cell count in expression value comparisons.

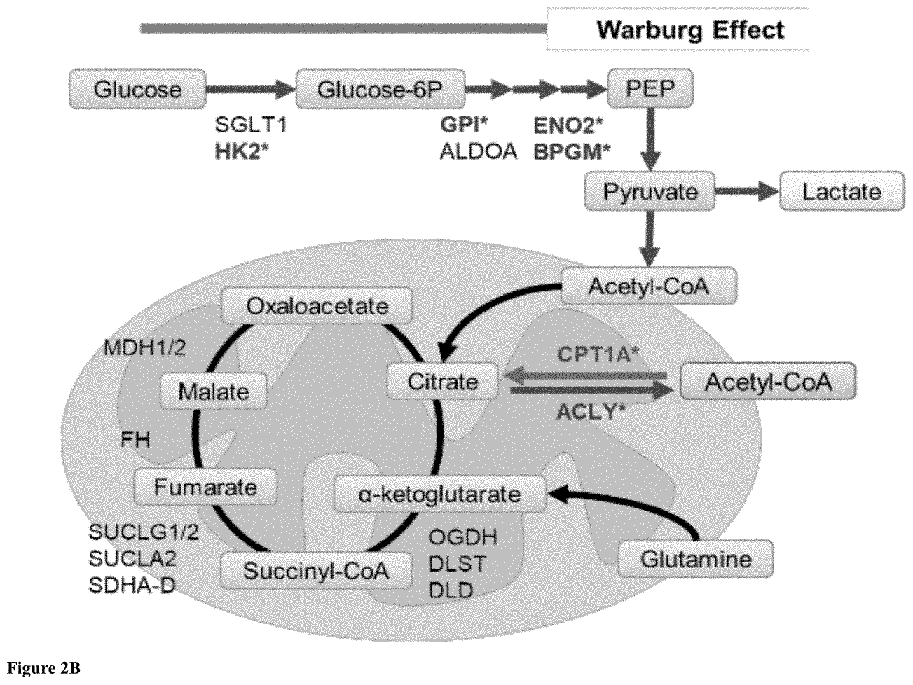

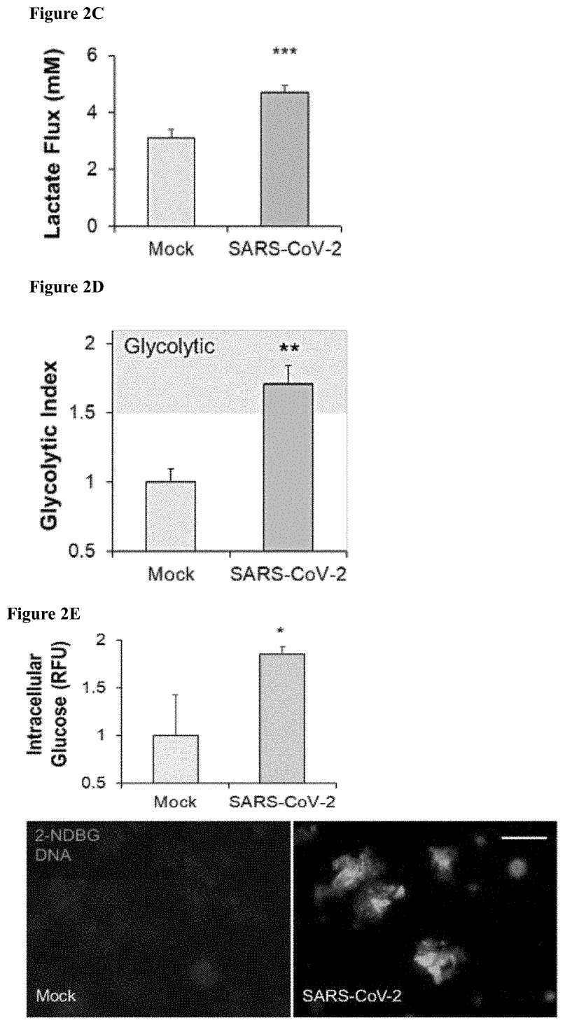

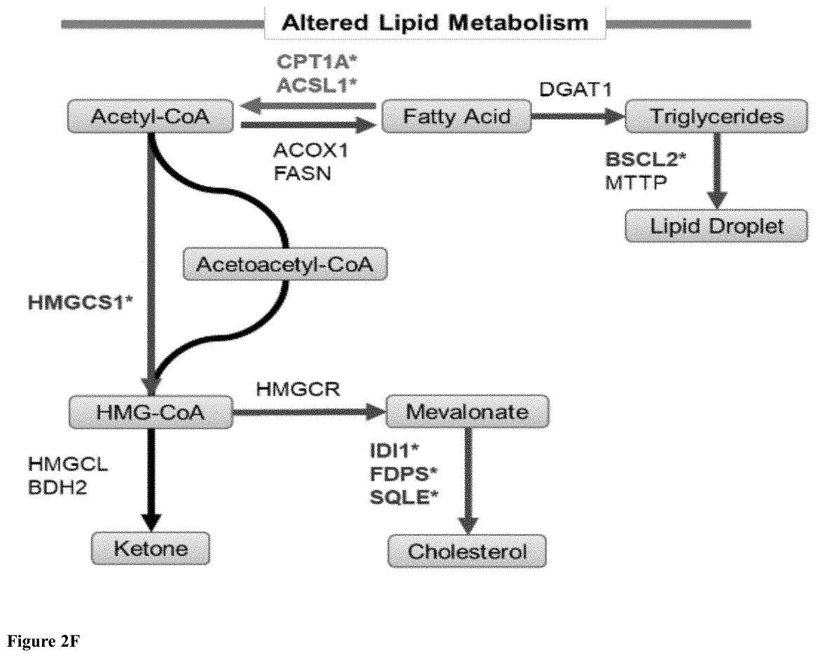

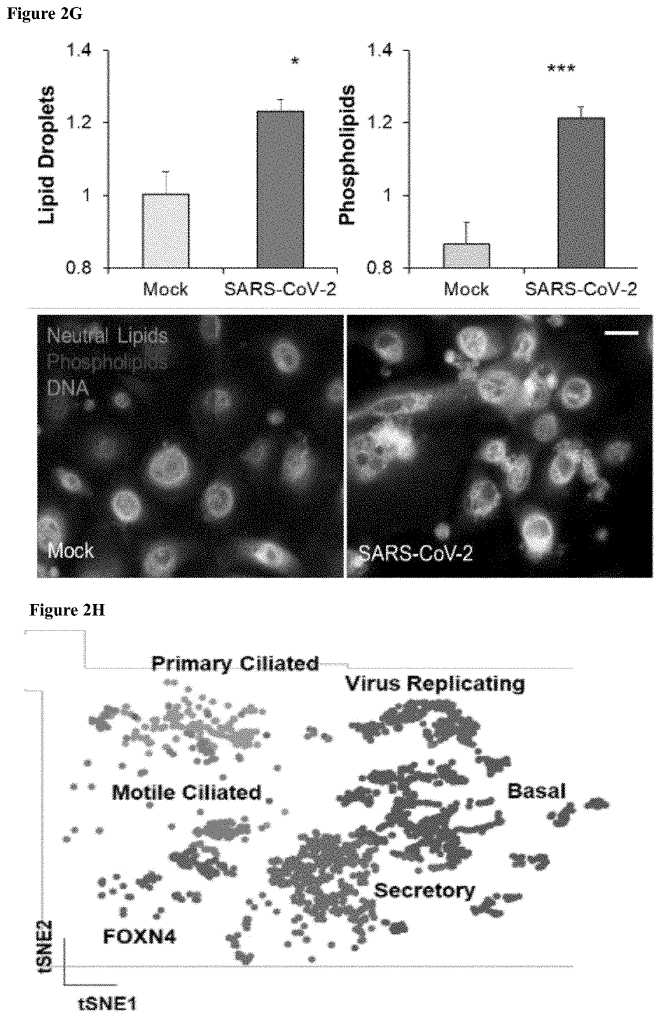

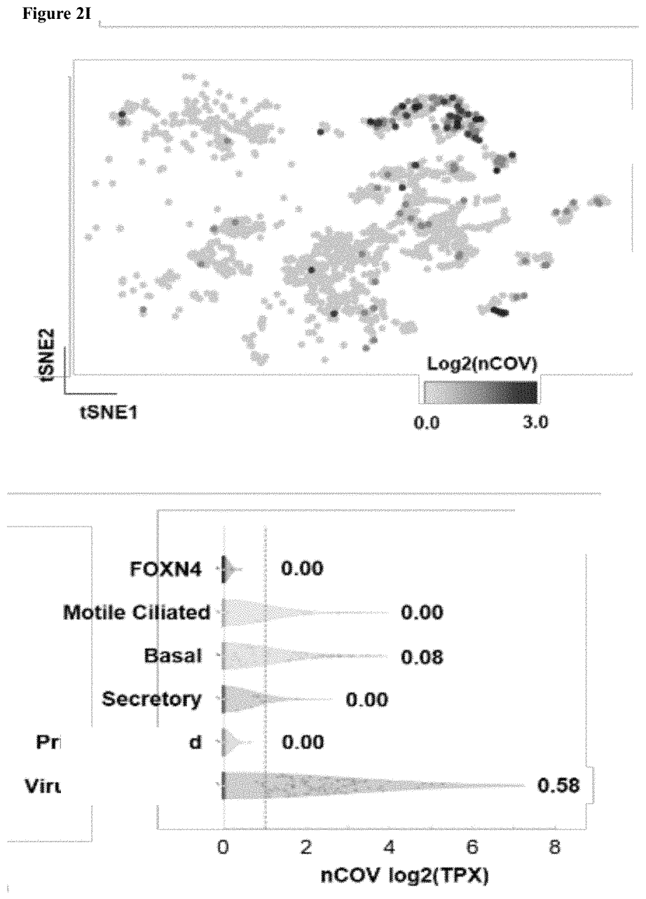

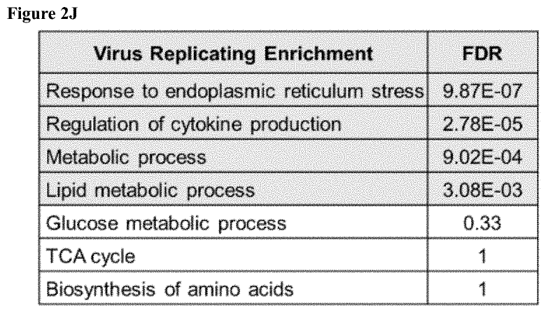

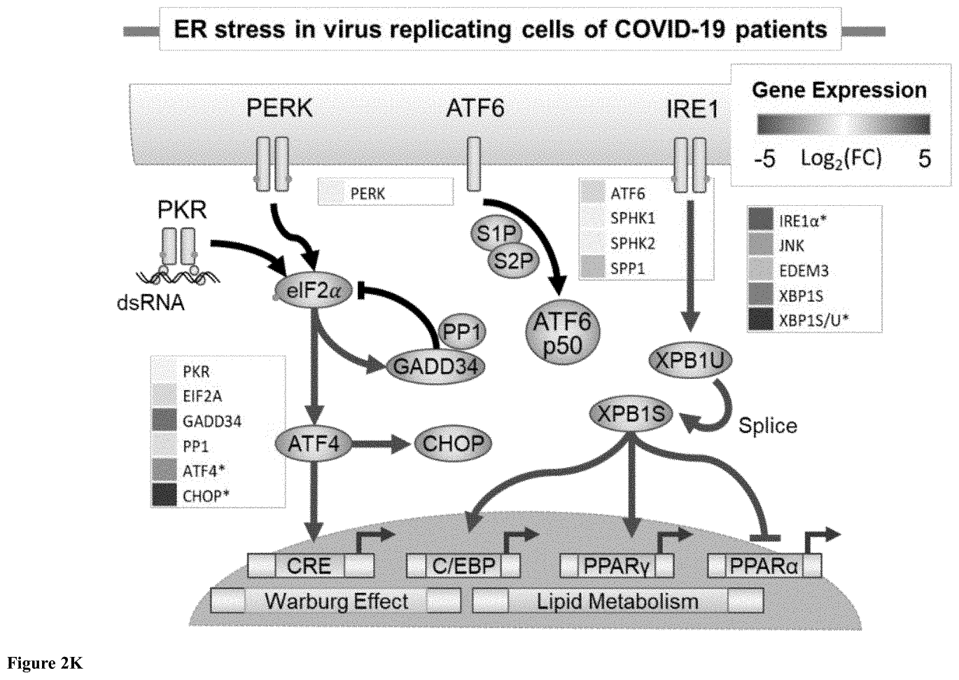

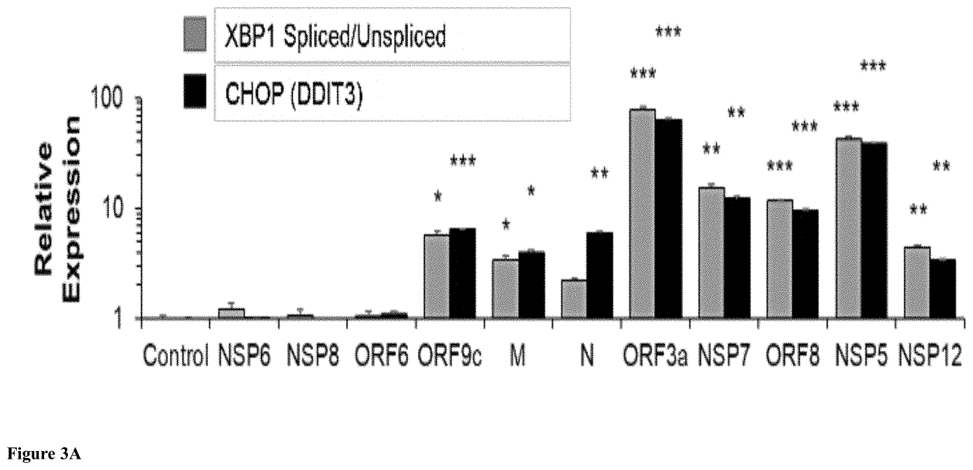

Identification of cell type and state: Epithelial cell types were primarily annotated based on their expression levels of their respective cell type markers. All previously described major epithelial cells of the conducting airways, including basal, secretory and ciliated cells, as well as the recently discovered FOXN4+ cells and ionocytes, were found. Basal, secretory, and ciliated cells were shown by their expression levels of respective cell type markers. Notably, the FOXN4+ cells resemble the transient secretory cell type described as the most SARS-CoV-2-vulnerable bronchial cells in non-infected individuals by virtue of their function as transitionary cell types differentiating from the secretory to ciliated lineage (FIG. 2A, 3A). Small clusters of cells that are predominantly derived from single patients that are called herein ‘Virus Replicating’ were also identified, which have a strong expression of SARS-CoV-2 markers.

Generation Lentiviral SARS-CoV-2 Constructs: Plasmids encoding the SARS-CoV-2 open reading frames (ORFs) and eGFP control are available from Addgene (plasmid #141367-141395). Plasmids were acquired as bacterial LB-agar stabs. Briefly, each stab was first seeded into agar LB (Bacto Agar; BD, USA) in 10 cm plates. Then, single colonies are inculcated into flasks containing LB (BD Difco LB Broth, Lennox; BD, USA) and 100 μg/ml penicillin (BI, Israel). Transfection grade plasmid DNA was isolated from each flask using the ZymoPURE II Plasmid Maxiprep Kit (Zymo Research, USA) according to the manufacturer's instructions.

HEK 293T cells (ATCC, USA) were seeded in 10 cm cell culture plates at a density of 4×106 cells/plate. The cells were maintained in 293T medium composed of DMEM high glucose (4.5 g/1; Merck, USA) supplemented with 10% FBS (BI, Israel), lx NEAA (BI, Israel), and 2 mM L-alanine-L-glutamine (BI, Israel).

The following day, the cells were transfected with a SARS CoV 2 orf-expressing plasmid and the packaging plasmids using the TransIT-LT1 transfection reagent (Minis Bio, USA). Briefly, 6.65 ug SARS CoV 2 lentivector plasmid, 3.3 μg pVSV-G, and 5 μg psPAX2 were mixed in Opti-MEM reduced serum medium (Gibco, USA), with 45ul of TransIT-LT1, and kept at room temperature to complex and then added to each plate. Following 18h of incubation, the transfection medium was replaced with 293T medium and virus-rich supernatant was harvested after 48h and 96h. The supernatant was clarified by centrifugation (500×g, 5 min) and filtration (0.45 um, Millex-HV, MerckMillipore). All virus stocks were aliquoted and stored at −80° C..

The packaging plasmids (psPAX2 and pVSV-G) were used.

SARS-CoV-2 Proteins Lentiviral Transduction: Approximately 1×105 cells were infected in two consecutive sessions of 12h each. A 50% dilution of the viral stock was used both for a final transduction efficiency of ˜60%. transduction efficiency was validated by microscopy of the eGFP transduced culture.

RNA-Seq of Viral Infections: Approximately 1×105 NHBE cells were infected with SARS-CoV-2 at a MOI of 2 for 24 h in complete bronchial epithelial growth media. Total RNA from infected and mock-infected cells was extracted using TRIzol Reagent (Invitrogen) and Direct-zol RNA Miniprep kit (Zymo Research) according to the manufacturer's instructions and treated with DNase I. RNA-seq libraries of polyadenylated RNA were prepared using the TruSeq RNA Library Prep Kit v2 (Illumina) according to the manufacturer's instructions. RNA-seq libraries for total ribosomal RNA-depleted RNA were prepared using the TruSeq Stranded Total RNA Library Prep Gold (Illumina) according to the manufacturer's instructions. cDNA libraries were sequenced using an Illumina NextSeq 500 platform.

Viral Load Quantitative Real-Time PCR Analysis: Genomic viral RNA was extracted from supernatants using TRIzol reagent (Thermo Fisher). RNA was reverse transcribed into cDNA using oligo d(T) primers and SuperScript II Reverse Transcriptase (Thermo Fisher). Quantitative real-time PCR was performed on a LightCycler 480 Instrument II (Roche) using KAPA SYBR FAST qPCR Master Mix Kit (KAPA biosystems) and primers specific for the SARS-CoV-2 nsp14 transcript. Forward: 5′-TGGGGYTTTACRGGTAACCT-3′ (SEQ ID NO: 1); Reverse: 5′-AACRCGCTTAACAAAGCACTC-3′ (SEQ ID NO: 2); Probe (in 5′-FAM/ZEN/3′-IBFQ format) 5′-TAGTTGTGATGCWATCATGACTAG-3′ (SEQ ID NO: 3); (W=A/T, Y=C/T, R=A/G). The viral load for each sample was determined using genomic viral RNA purified from viral stocks to generate a standard curve. Error bars indicate the standard error from three biological replicates.

Assembly of Metabolic Categories: Aggregate metabolic categories were created. Briefly, functional annotation gene-sets, taken from GO and KEGG, were merged into a set of glucose, lipid, mitochondrial, and amino acid gene-sets.

Metabolic Flux Quantification (Seahorse): Mitochondrial Stress Test assay was conducted. Briefly, cells were incubated in unbuffered DMEM supplemented with 2 mM glutamine, 1 mM sodium pyruvate, and 10 mM glucose (pH 7.4) for 1 hour at 37° C. in a non-CO2 incubator. Basal oxygen consumption rate (OCR) was measured for 30 min, followed by injection of 1.5 μM oligomycin, a mitochondrial Complex V inhibitor that blocks oxidative phosphorylation. The decrease in OCR due to oligomycin treatment is defined as the oxidative phosphorylation rate. 0.5 μM carbonyl cyanide-4 (trifluoromethoxy) phenylhydrazone (FCCP), an uncoupling agent, is added at 60 min to measure maximal mitochondrial activity followed by complete inhibition at 90 min using a mixture of 0.5 μM antimycin A and rotenone, mitochondrial Complex III and Complex I inhibitors.

Functional Annotations of Gene Expression: Differentially expressed genes were tested for enrichment overlap within functional gene sets. The general test for functional enrichment of the differentially expressed genes against various functional categories was done using the PANTHER tool. Enrichment P values were calculated using Fisher's exact test and corrected with familywise (Bonferroni) multiple hypotheses correction or Benjamini & Hochberg False discovery procedure as indicated.

Transcription Factor Target Genes: Transcription factor (TF) gene targets were aggregated from the following databases: MsigDB v7.1, Trrust TF database v2, RegNetwork. Enrichment of TF targets among differentially expressed genes, stratified by selected metabolic gene categories (see above) was tested with a hypergeometric enrichment test, and adjusted for false discovery rate using a Benjamini & Hochberg procedure.

Drug Treatments: Approximately 5×105 NHBE cells were infected with SARS-CoV-2 at a MOI of 2 in bronchial epithelial growth media. Culture media was supplemented with 0.1% DMSO (vehicle control), 10 μM Cloperastine (Merck; C2040), 5 μM Empagliflozin (AG-CR1-3619), 1 mM Metformin (Merck; 317240), 20 μM Fenofibrate (Merck; F6020), 20 μM Rosiglitazone (Merck; R2408) or 10 μM GW9662 (Merck; M6191). After 24 h, the media was collected and changed to bronchial epithelial growth media with the respective drug at the concentration listed above. Then, every 48 hours media was collected and replenished. The media was stored at −80° C. immediately after removal. Culture viability was assessed at the end the experiment using Hoechst staining.

Israeli Study Design and Participants: A retrospective, multi-center study was conducted in Hadassah and Ichilov medical centers. A total of 20,153 participants were diagnosed positive for SARS-COV-2 following WHO interim guidance (World Health Organization, 2020). Only patients hospitalized and diagnosed with COVID-19 were included. Participants with incomplete electronic medical records, aged less than 18, with pregnancy or severe medical conditions, including acute lethal organ injury (i.e., acute coronary syndrome, acute stroke, and severe acute pancreatitis) were excluded. The flowchart for patient inclusion is illustrated in FIG. 4J. Participants were admitted between Mar. 1, 2020, and Aug. 31, 2020 to either the Hadassah Medical Center in Jerusalem or the Tel Aviv Sourasky Medical Center. The final date of the follow up was Sep. 28, 2020. The study protocols were approved by the institutional ethics committee. Patient informed consent was waived by each ethics committee. Demographic and clinical characteristics, vital signs, laboratory tests, radiological reports, therapeutic interventions, and outcome data were extracted from electronic medical records using a standardized data collection. The laboratory data included a routine blood test, serum biochemical markers reflecting liver injury, kidney injury, and cardiac injury, lipid profile, IL-6, CRP, procalcitonin, and D-dimer were collected during hospitalization. In-hospital medication and life support intervention included the classification of the drugs, the dosage, the course of treatment, and using respiratory support were also extracted from medical records.

End Point Definition: The primary endpoint was defined as 28-day all-cause mortality. The secondary endpoints were the occurrence of septic shock, acute liver injury, acute kidney injury, acute cardiac injury, invasive mechanical ventilation, and intensive care unit admission. Septic shock was defined according to the WHO interim guideline “Clinical management of severe acute respiratory infection when novel coronavirus (2019-nCoV) infection is suspected.” Acute kidney injury was diagnosed by an elevation in serum creatinine level ≥26.5 μmmol/L within 48 h. Acute cardiac injury was defined with a serum level of cardiac troponin T (cTnT) above the ULN. Acute liver injury was defined using serum ALT or alkaline phosphatase above 3 folds of ULN.

Propensity Score-Matched Analysis: In the Israeli Study, to minimize baseline differences between treatment and non-treatment groups, propensity score-matched analysis (PSM) was performed. Baseline matching variables included age, gender, clinical characteristics on admission (heart rate, blood pressure, oxidation, and temperature), pre-existing comorbidities (smoking, asthma, COPD, DM, hypertension, diabetes, coronary heart disease, obesity, dyslipidemia, cerebrovascular disease, chronic liver disease, and chronic kidney disease) and indicators of disease severity and organ injuries on admission (platelets, neutrophil, lymphocyte counts, and c-reactive protein, cardiac troponin, ferritin, creatinine, LDH, d-dimer, bilirubin, lactic acid, and glucose levels). Users and nonusers were paired according to the propensity scores using exact matching with a caliper size of 0.1. The balance of covariates was evaluated by estimating standardized differences before and after matching, and a small absolute value of less than 0.1 was considered qualified balancing between the two groups. The treatment versus non-treatment group ratio was paired at 1:1,1:5 or 1:10 according to treatment group size.

In the American Study to minimize baseline differences between treatment and non-treatment groups, a 1:5 propensity score matching was performed using SPSS statistics 23.0. Baseline matching variables included age, sex, body mass index, race/ethnicity, and history of atherosclerotic cardiovascular disease, heart failure, diabetes mellitus, chronic lung disease, chronic liver disease, dementia, and current or former smoker. Nearest neighbor matching was performed with a caliper of 0.1. A <10% standardized difference in each of the matched covariates between matched groups, as well as Rubin's B of ≤25% and Rubin's R between 0.5-2 was required to verify sufficient matching.

Mixed-Effects Cox Model: The risk of primary and secondary endpoints and the corresponding hazard ratio (HR) were calculated using the Cox proportional hazard model comparing the treatment group versus the non-treatment group. In the Cox analysis, individuals discharged were treated as “0—at risk” but not censored data as individuals with COVID-19 would not be discharged unless their symptoms were significantly relieved and two continuous viral PCR negatives were achieved. Any death that occurred was documented. Thus, discharged individuals were unlikely to die due to COVID-19 and their survival information was still available after discharge.

Regression adjustment was applied to remove post-PSM residual confounding bias where it included the covariates with a standardized difference greater than 0.10. Multi-variable adjusted residual imbalances including age, gender, clinical characteristics on admission, indicators of disease severity and organ injuries on admission, pre-existing diseases, and other treatments (metabolic regulators, ACEi, dexamethasone, and remdesivir) were performed when analyzing the association between treatment and clinical outcomes. The proportional hazard assumptions were verified using correlation testing based on the Schoenfeld residuals.

Association of metabolic regulators with COVID-19 Mortality: To test the association between in-hospital metabolic regulator therapy and mortality, Cox proportional hazards regression model was applied after propensity score-matching for baseline characteristics, but without considering immortal time bias or time-varying confounders.

Missing Data and Imputation: Variables were used for matching in propensity-score matched analysis and for adjusting in Cox analysis at admission. To account for the missing data on the laboratory variables, non-parametric missing value imputation was used, based on the missForest procedure in the R. A random forest model based on the rest of the variables in the dataset was constructed to predict the missing values with an estimation of the internally cross-validated errors.

Quantification and Statistical Analysis: All experiments were done in at least 3 biological repeats. Measurements were done in either technical triplicates or quadruplets, images were analyzed with 5 or more fields of view; Graphs show mean±SEM; Continuous variables were calculated using Mann-Whitney Rank Sum or Student's t-test, categorical variables with chi-square, and ANOVA tests.

Pairwise comparisons were performed using Student's t-test; Mann-Whitney U test was used when the distribution could not be determined to be normal; FDR correction was used to adjust for multiple comparisons and RNA seq comparisons; Hypergeometric testing was used to assess statistically significant enrichments. * indicates p<0.05, ** indicates p<0.01, *** indicates p<0.001, unless denoted otherwise.

In clinical data, continuous variables with non-normal distributions were expressed as median [IQR]. Categorical variables were expressed as number and percentage (%). The sample size is detailed in each display item. Comparisons between groups were performed with Mann-Whitney U test for nonparametric variables and Fisher's exact test or χ2 test for categorical variables. Person-time data (Incidence) of two groups with different exposures may be expressed as a difference between incidence rates or as a ratio of incidence rates (IRRs). The IRRs of endpoint outcomes were calculated to estimate the incidence difference in absolute change in the incidence of two comparison groups. The cumulative rates of death were compared using the Kaplan-Meier curves. Dynamic changes of inflammatory factors tracking from day 0 to day 28 after admission were depicted using the Lowess model. A two-side a less than 0.05 was considered to define statistical significance. Data were analyzed in R-3.6.3 (R Foundation for Statistical Computing, Vienna, Austria) and SPSS Statistics (version 23.0, IBM, Armonk, N.Y., USA).

### Example 1: The Metabolic Fingerprint of SARS-CoV-2 Infection

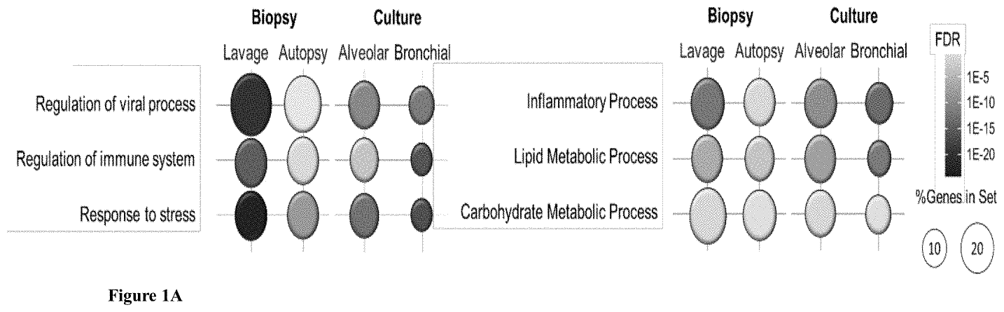

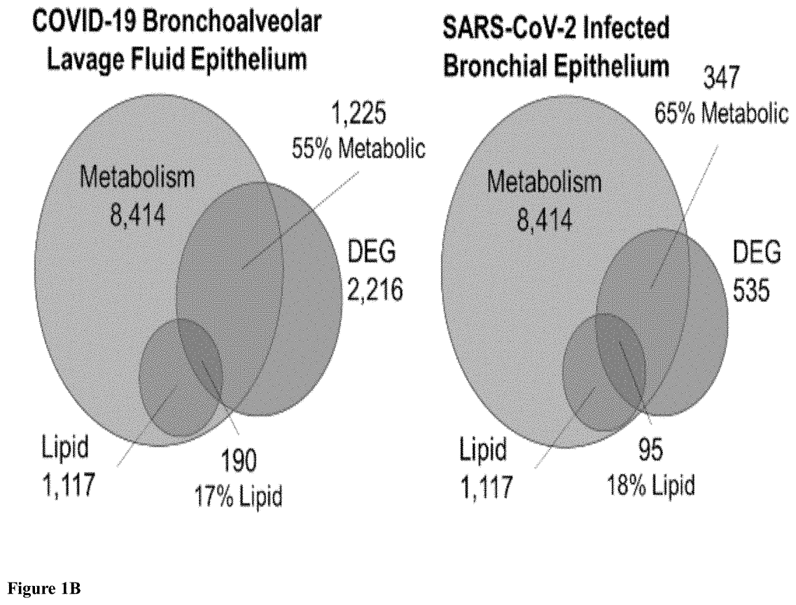

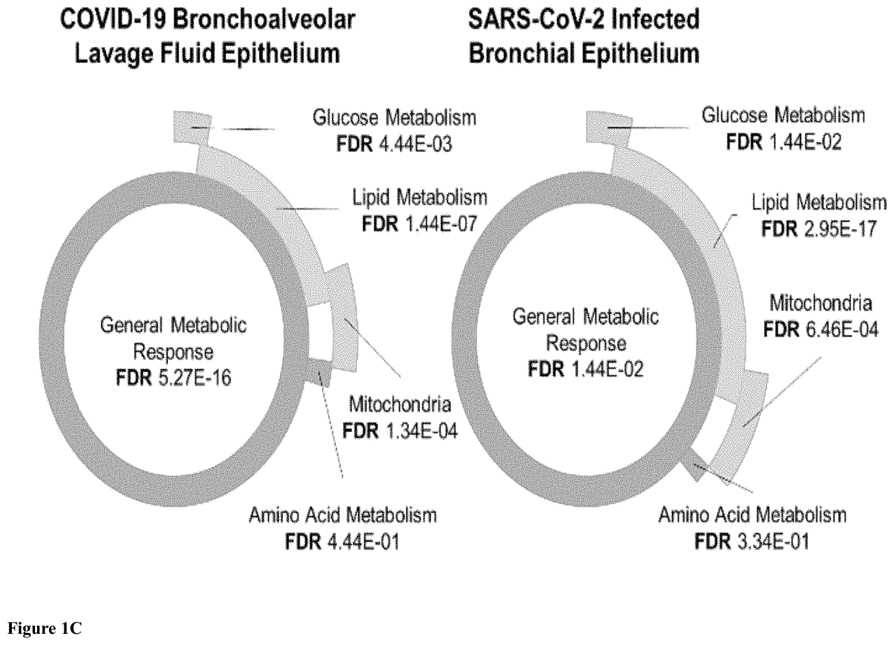

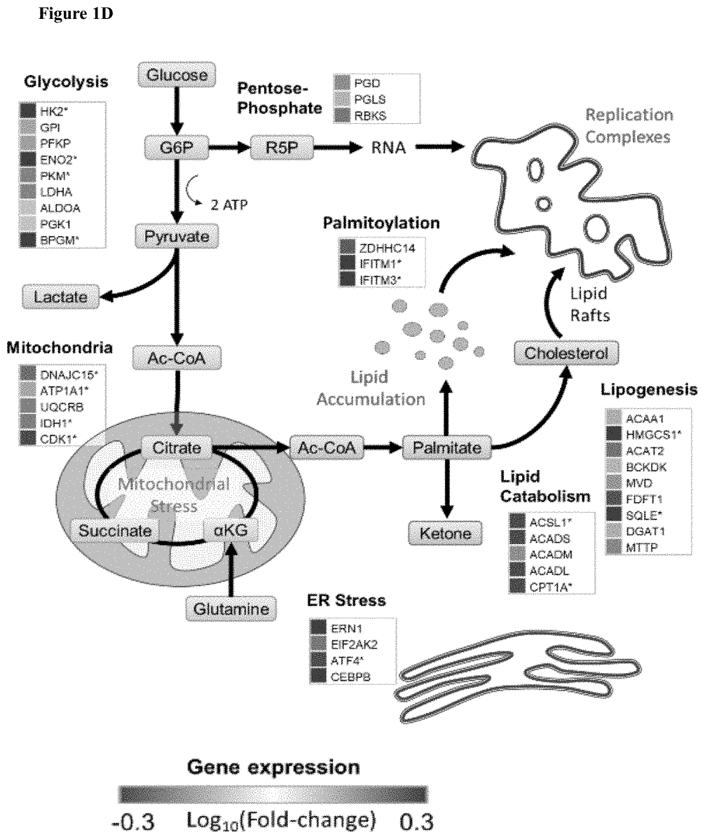

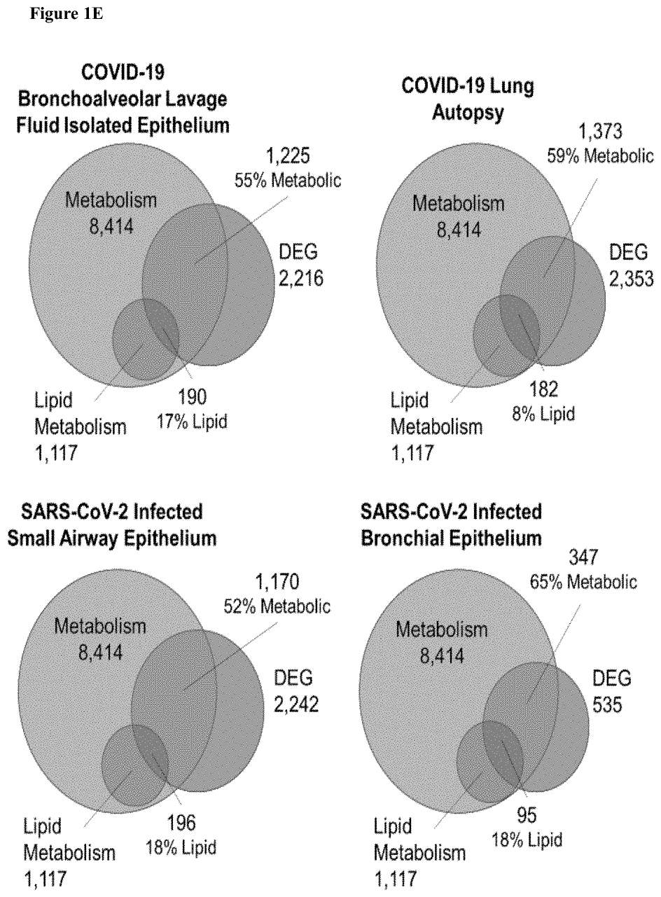

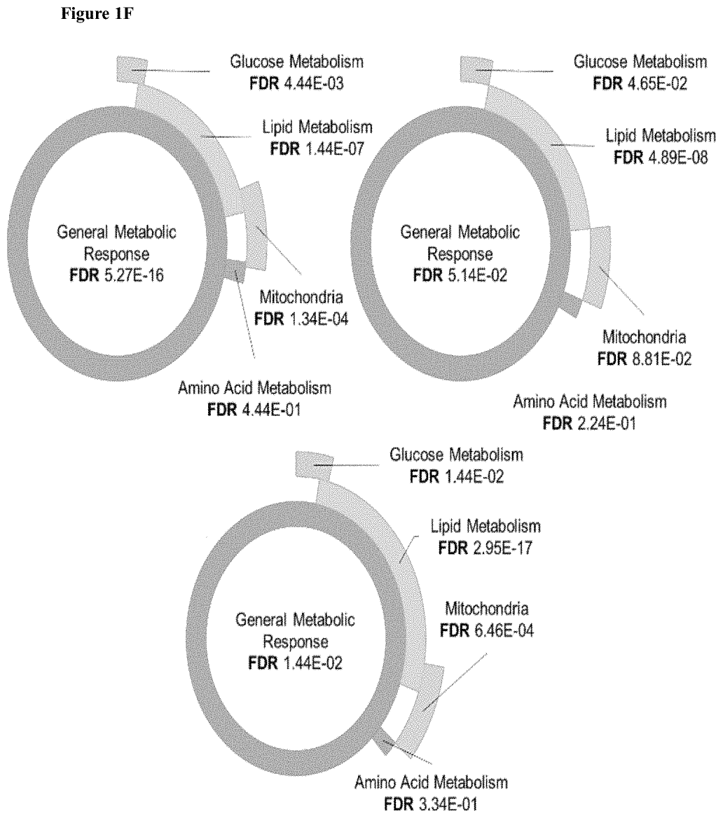

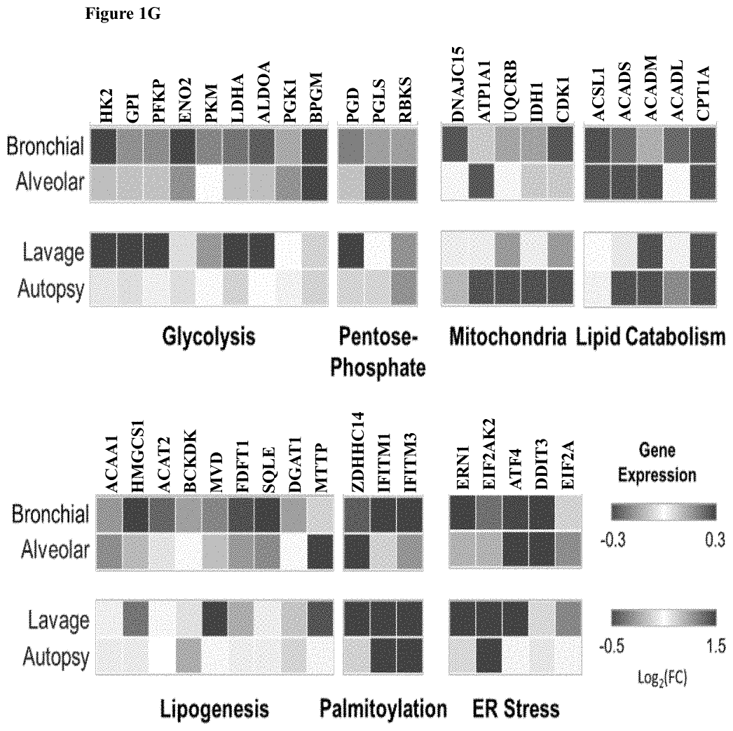

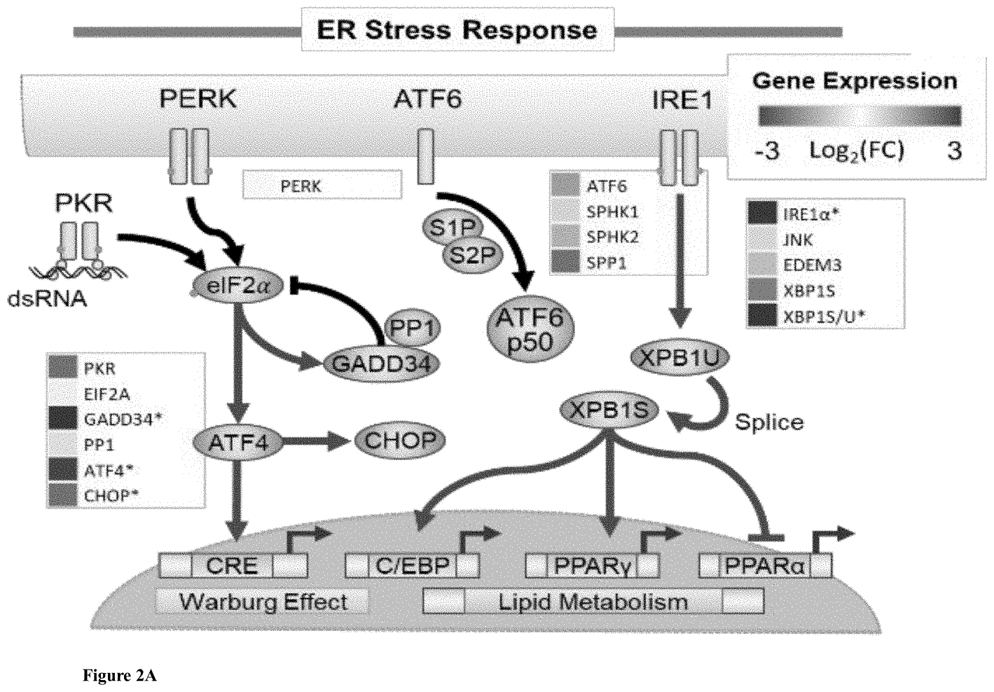

To elucidate the metabolic effects of SARS-CoV-2 primary human bronchial epithelial cells were infected with the virus (see Materials and Methods). Infected cells became noticeably smaller, showing vacuolization. RNA-Seq analysis of infected primary cells identified 535 differentially expressed genes (FDR<0.05). Enrichment analysis identified the regulation of viral transcription (FDR<3×10⁻²), immune processes (FDR<9×10⁻⁴), and cellular response to stress (FDR<5×10⁻¹¹). An analysis was also carried out on RNA-Seq data obtained from primary small airway epithelial cells infected with SARS-CoV-2, as well as epithelial cells isolated by bronchoalveolar lavage and lung biopsies taken from COVID-19 patient autopsies. All samples showed similar enrichment patterns (FIG. 1A).

Interestingly, all samples showed changes in metabolic processes (FDR<4×10⁻⁴), particularly lipid (FDR<2×10⁻⁵) and carbohydrate metabolic processes (FDR<0.05; FIG. 1A). About 58±3% of the differentially expressed genes were metabolism-related, with 15±2% of the genes associated with lipid metabolism (FIG. 1B, 1E-G). Analysis of aggregated metabolic categories showed similar enrichment of lipid metabolism (FDR<3×10⁻¹⁷), mitochondrial function (FDR<7×10⁻⁴), and glucose metabolism (FDR<2×10⁻²) in both primary cultures and patient samples (FIG. 1C, 1E-G).

Mapping SARS-CoV-2 induced transcriptional changes on the metabolic landscape of lung epithelial cells showed induction of a Warburg-like effect observed in other viruses and suggested to provide nucleotides for viral replication. Lipogenesis was upregulated, supporting palmitoylation of viral proteins as well as lipid components of the viral replication complex (FIG. 1D). However, in contrast to other viruses, SARS-CoV-2 infection appears to downregulate lipid catabolism (FIG. 1D, 1E-G).

### Example 2: Metabolic Effects of SARS-CoV-2 Replication

RNA-Seq analysis showed a significant cellular response to stress (FDR<5×10⁻¹¹), particularly genes associated with endoplasmic reticulum (ER) stress (FIG. 2A). SARS-CoV-2 infection of primary cells induced the dsRNA-activated protein kinase R (PKR/PERK) and IRE1 pathways leading to differential expression of ATF4 and the splicing of XBP1. ATF6 pathway of ER stress was seemingly unaffected by the infection. Induction of PKR/PERK and IRE1 pathways are known to lead to a Warburg-like shift to anaerobic glycolysis and increased lipogenesis (FIG. 2A).

Mapping differentially expressed genes on the central carbon metabolism pathway showed SARS-CoV-2 induction of key glycolysis genes (FIG. 2B) including rate-limiting enzymes such as hexokinase 2 (HK2) and pyruvate kinase isozyme (PKM). Metabolic analysis of SARS-CoV-2 infected cells supports these findings, showing a 50% increase (n=6, p<0.001) in lactate production (FIG. 2C) and a shift in the lactate over glucose ratio (glycolytic index) from 1 to 1.7 indicating a Warburg-like effect. Microscopic analysis confirmed a marked 85% increase in intracellular glucose in infected cells (FIG. 2E). Interestingly, while core genes of the citric acid cycle didn't change significantly, ATP citrate lyase (ACLY) was upregulated indicated a shift toward fatty acids synthesis.

Similar mapping of differentially expressed genes on the lipid metabolism pathways (FIG. 2F) showed induction of HMG-CoA synthase (HMGCS) and squalene monooxygenase (SQLE) rate-limiting steps in cholesterol synthesis. Surprisingly, only a few significantly upregulated lipogenesis genes were found, but rather significant down-regulation of lipid catabolism genes CPT1A and ACSL1 were found (n=3, FDR<0.01) (FIG. 2F). Fluorescence microscopy confirmed a significant perinuclear accumulation of neutral lipids (n=3, p<0.05) and phospholipids (n=3, p<0.001) in infected cells (FIG. 2G).

These in vitro data suggest that metabolic changes may be induced by SARS-CoV-2 infection separately from the immunoinflammatory disease. To validate these findings in clinically relevant data, single-cell transcriptional profiles of 2,629 epithelial cells obtained from 6 symptomatic COVID-19 patients were analyzed (FIG. 2H-I). The analysis identified 6 different transcriptional signatures including basal, secretory, and ciliated lung cells as well as a cluster of virus-replicating cells (FIG. 2I, 2K-L). Gene enrichment analysis comparing virus replicating cells to uninfected epithelial cells in the same patients showed a response to endoplasmic reticulum stress (FDR<1×10⁻⁶), metabolic processes (FDR<9×10⁻⁴), and lipid metabolism (FDR<3×10⁻³; FIG. 2J). The single-cell analysis showed no significant transcriptional differences in glycolysis or mitochondrial metabolism, suggesting these pathways are not uniquely induced by viral replication.

### Example 3: SARS-CoV-2 Proteins Cause Direct Modulation of Metabolic Pathways

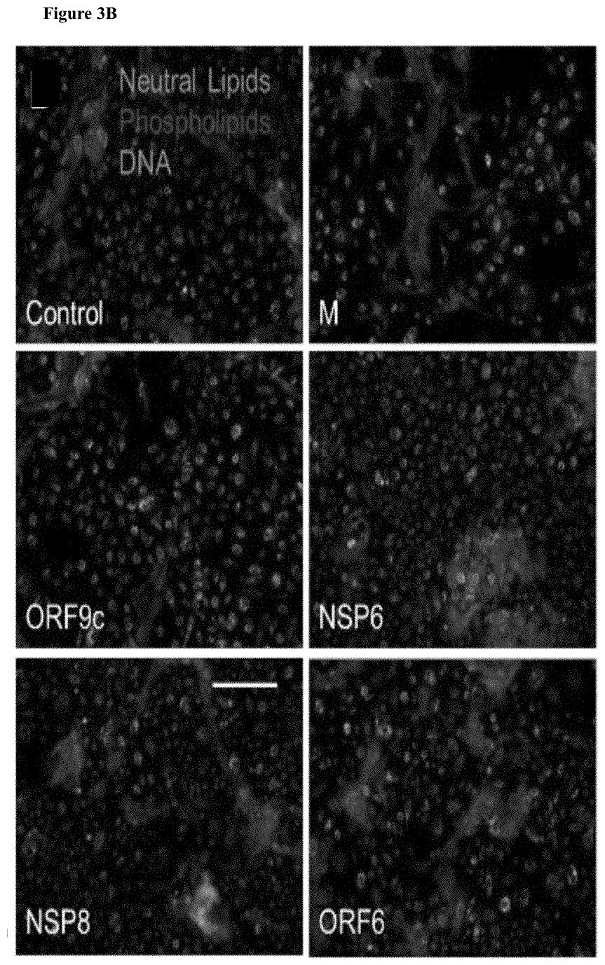

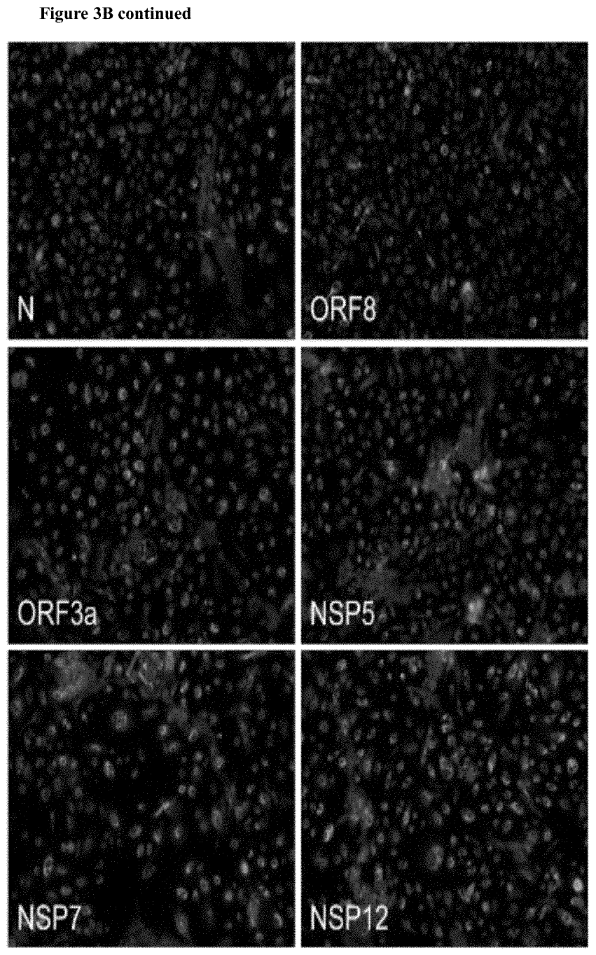

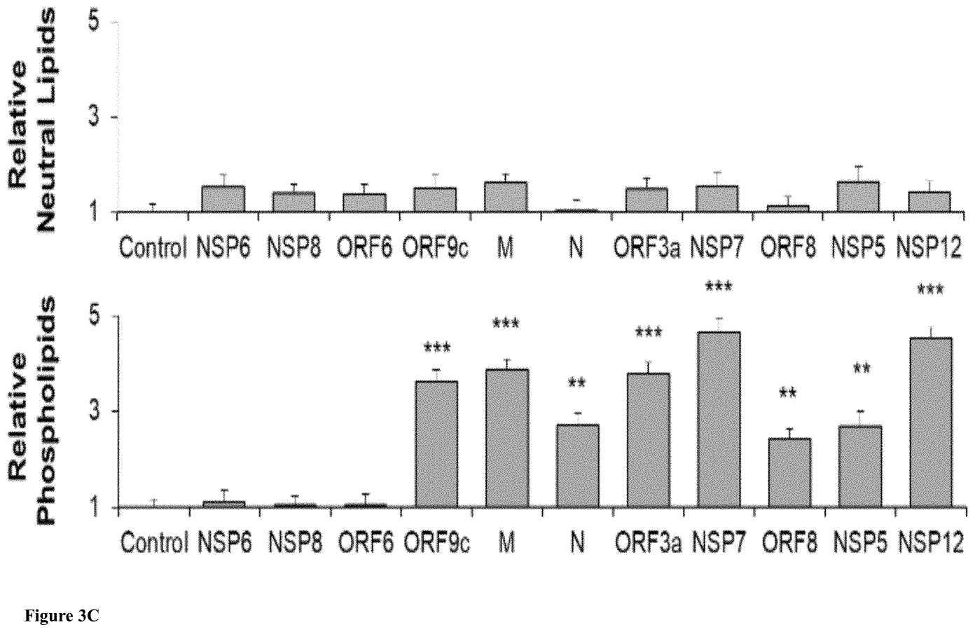

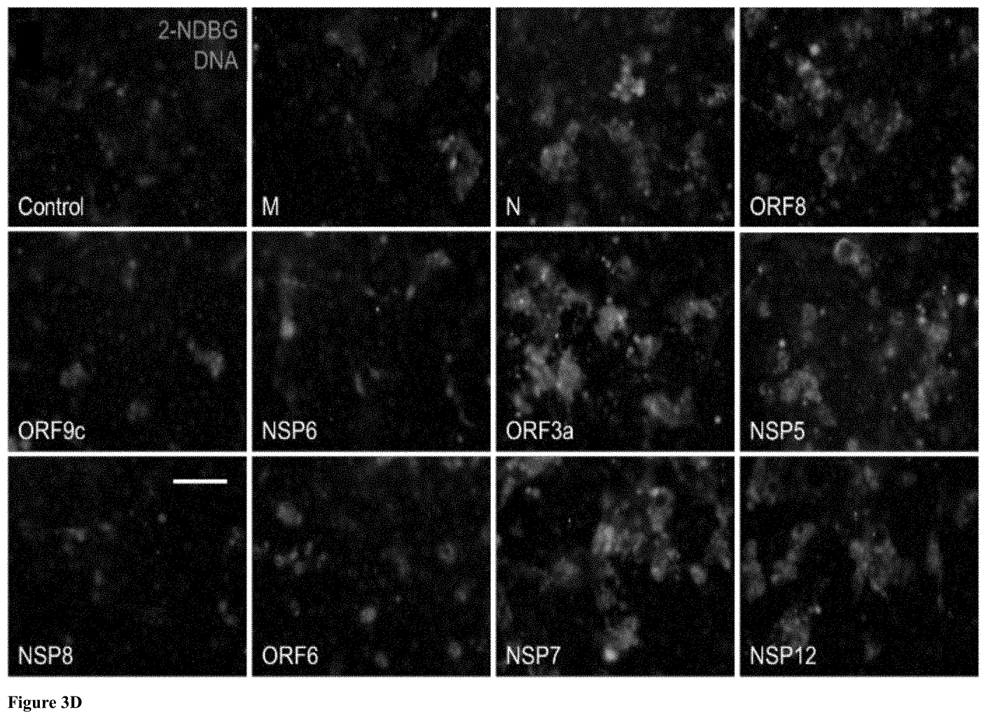

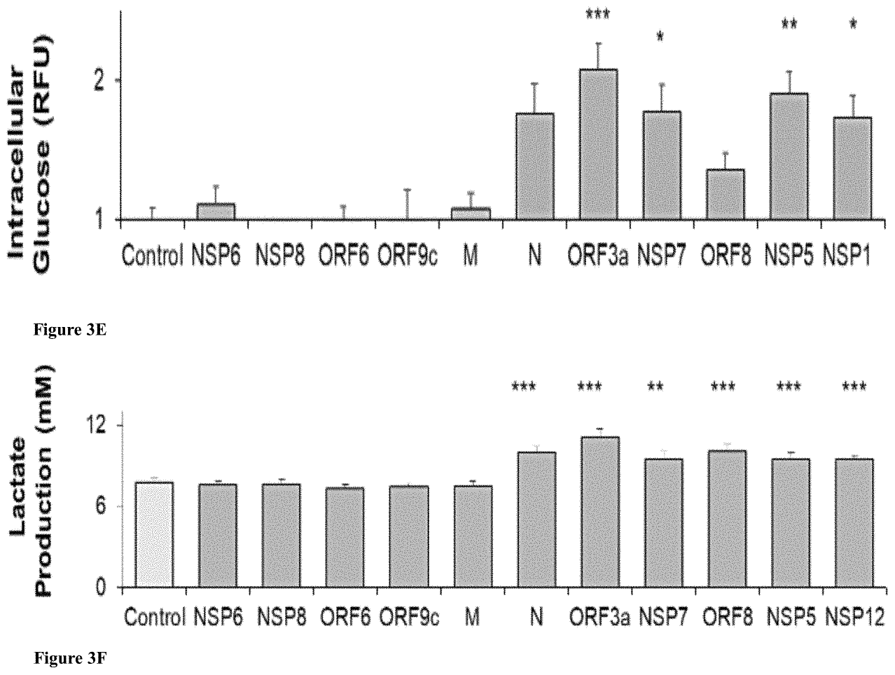

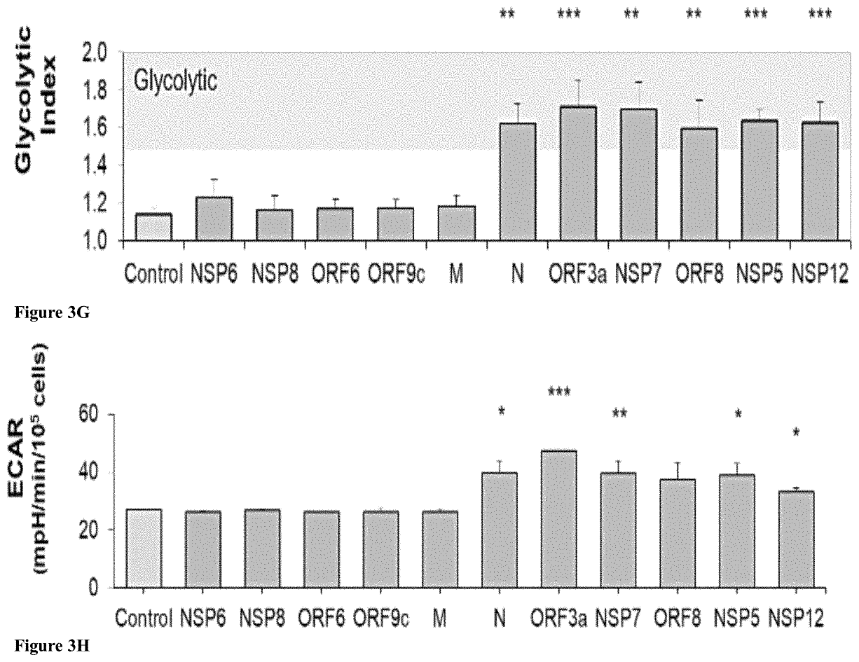

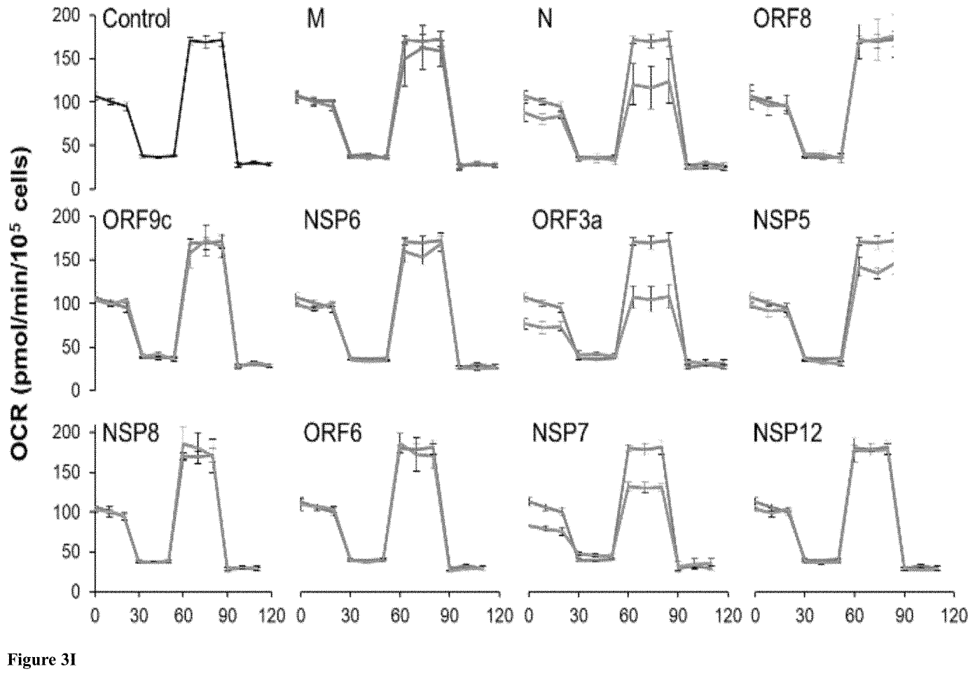

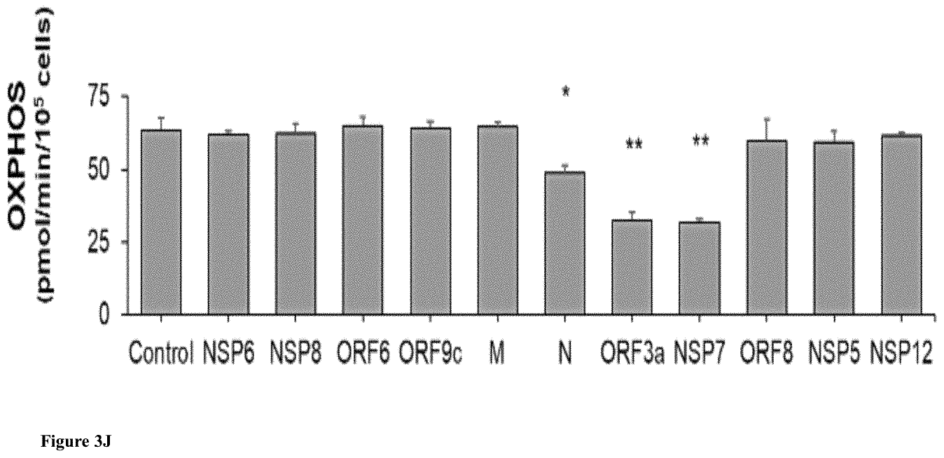

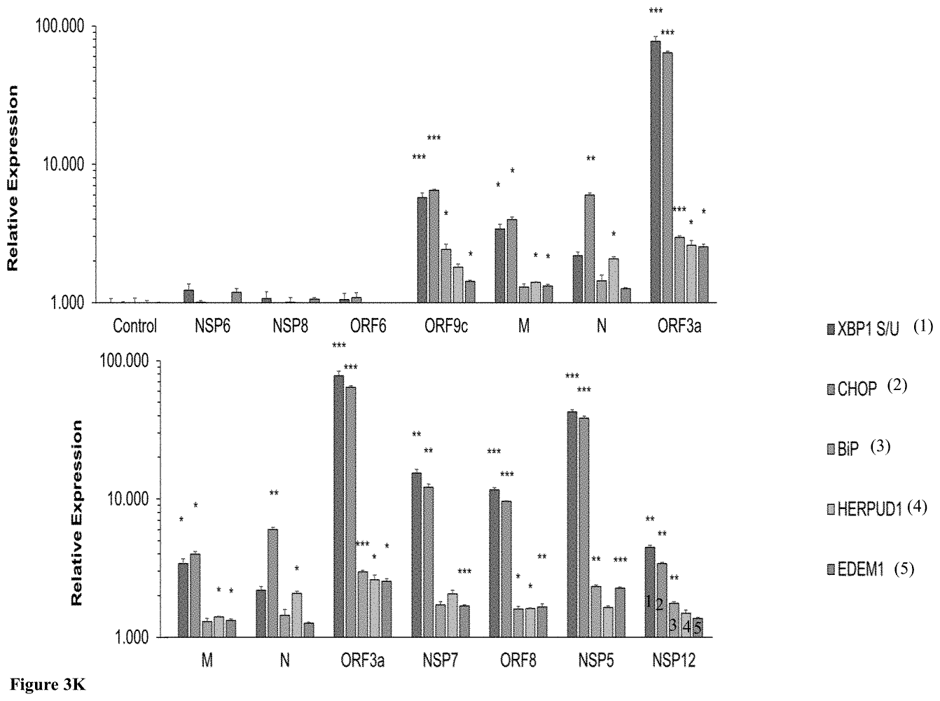

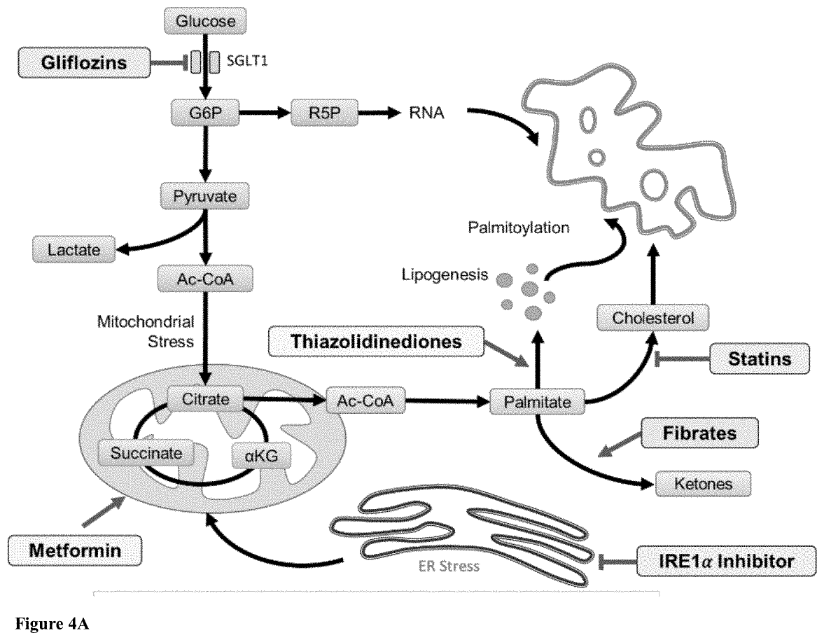

To analyze the role of viral proteins in the host metabolic response to SARS-CoV-2 a large protein panel was expressed in primary bronchial epithelial cells. Gene expression analysis showed a strong up-regulation of CHOP and XBP1 splicing induced by expression of ORF9c, M, N, ORF3a, NSP7, ORF8, NSP5, and NSP12 (n=6, p<0.05; FIG. 3A, 3K), supporting activation of PKR/PERK and IRE1 pathways (FIG. 2A). Microscopic analysis showed significant accumulation of phospholipids induced by expression of the same viral proteins: ORF9c, M, N, ORF3a, NSP7, ORF8, NSP5, and NSP12 (n=6, p<0.01; FIG. 3B-C) supporting a link between SARS-CoV-2 induced ER stress and lipid accumulation.

Microscopic analysis of glucose accumulation showed the involvement of a smaller subset of the same viral proteins including N, ORF3a, NSP7, ORF8, NSP5, and NSP12 (FIG. 3D-E). In addition, direct measurement of glucose uptake and lactate production showed a marked increase in lactate production in cells expressing the same viral proteins: N, ORF3a, NSP7, ORF8, NSP5, and NSP12 (n=6, p<0.05; FIG. 3F) confirming a viral protein-driven shift to glycolysis (FIG. 3G). Independent measurement of extracellular acidification rate (ECAR), a surrogate measurement for glycolysis, confirmed the activity of the same viral proteins (FIG. 3H)

Finally, mitochondrial stress test analysis (see Materials and Methods) showed a marked disruption in oxidative phosphorylation, induced by expression of N, ORF3a, and NSP7 (n=6, p<0.05; FIG. 3G-H), which appear to play a role in the measured changes in all three metabolic pathways.

### Example 4: Pharmaceutical Modulation of SARS-CoV-2 Induced Metabolic Pathways

The metabolic pathways induced by SARS-CoV-2 infection can be pharmaceutically modulated at multiple points (FIG. 4A-B). SGLT inhibitors like empagliflozin can block glucose absorption, while metformin can increase mitochondrial activity potentially reversing a Warburg-like effect. Cholesterol synthesis can be blocked by HMGCR inhibitors like statins, while lipid oxidation can be induced by fibrates. Thiazolidinediones act by increasing lipid synthesis and thus are expected to produce a worse outcome, while telmisartan could act by decreasing ER stress through IRE1 inhibition.

Exposing SARS-CoV-2 infected primary cells to physiological concentrations (Cₘₐₓ) of these drugs produced mixed effects (FIG. 4C-G). Rosiglitazone, empagliflozin, and metformin showed no effect at the concentrations studied. Cloperastine (Hustazor), a recently identified SGLT1 inhibitor, reduced viral load by 3-fold (p<0.01) without affecting cell viability but did not result in a reduction of lipid content or change in the glycolytic index. However, PPARα agonist fenofibrate (Tricor®) blocked phospholipid accumulation (p<0.001) and the increase in glycolysis (FIG. 4C-G). A 5-day treatment with fenofibrate reduced viral load by 2-logs (p<0.001) without affecting cell viability (FIG. 4F-I). Other PPARα agonists, including bezafibrate, WY14643, and conjugated linoleic acid (CLA) showed a similar effect (FIG. 4H-I).

To validate these data in a clinical setting a total of 1,531 cases of confirmed COVID-19 patients admitted to Hadassah and Ichilov Medical Centers between March to July 2020 were gathered. More than 500 patients were registered with in-hospital use of different metabolic regulators (FIG. 4J). Participants treated with metabolic regulators were older and had a higher prevalence of chronic medical conditions, including hypertension, diabetes mellitus, coronary heart disease, cerebrovascular diseases, and chronic kidney diseases than those without these treatments (Table 2) and thus were expected to be over-represented in ICU admissions and COVID-19 related deaths. A chi-squared test comparing COVID-19 patients to 249,939 recent unique hospital patient records show significant over-representation of patients taking thiazolidinediones and metformin across all severity indicators, while patients taking SGLT2 inhibitors, statins, or telmisartan (IRE1α inhibitor) were over-represented in ICU admission and deaths (FIG. 4H, Table 3). While patients taking fibrates shared the same risk factors (Table 2), they were not over-represented across all severity indicators (FIG. 4H, Table 3).

TABLE 2
Characteristics of metabolic regulators users before PSM. SBP, systolic blood pressure; DBP,
diastolic blood pressure; COPD, chronic obstructive pulmonary disease; SpO2, oxygen saturation;
ECMO, extracorporeal membrane oxygenation; IQR, interquartile range; SD, standardized difference.
P values were calculated by Mann-Whitney U test for non-normally distributed continuous
variables and Fisher's exact test or χ2 test for categorical variables.

| | | | |
|---|---|---|---|
| | Fibrates | Non-fibrates | p |
| Parameters | (N = 13) | (N = 1,519) | value |

Clinical Characteristics on Admission

| | | | | | |
|---|---|---|---|---|---|
| Age, median (IQR) | 69.7 | (57.45-74.4) | 52.90 | (33.6-67.6) | <0.001 |
| Female gender, n (%) | 6 | (46.2%) | 707 | (46.8%) | 0.855 |
| Heart rate, median (IQR), bpm | 81 | (73.5-90.5) | 85.00 | (74.0-96.0) | 0.146 |
| SBP, median (IQR), mmHg | 139.5 | (124.5-146) | 125.0 | (112.0-138.0) | 0.038 |
| DBP, median (IQR), mmHg | 60 | (58-83.5) | 73.0 | (65.0-82.0) | 0.025 |
| SpO2, median (IQR) | 94 | (93-95.5) | 97.0 | (94.0-99.0) | 0.008 |
| Temperature (P.O), median (IQR) | 37 | (36.65-37.3) | 36.9 | (36.7-37.4) | 0.139 |
| Smoking, n (%) | 0 | (0%) | 55 | (3.6%) | <0.001 |

Comorbidities on Admission

| | | | | | |
|---|---|---|---|---|---|
| Asthma, n (%) | 0 | (0%) | 48 | (3.2%) | <0.001 |
| COPD, n (%) | 0 | (0%) | 36 | (1.1%) | <0.001 |
| Hypertension, n (%) | 10 | (76.9%) | 407 | (30.3%) | <0.001 |
| Diabetes, n (%) | 8 | (61.5%) | 299 | (14.6%) | 0.024 |
| Coronary heart disease, n (%) | 3 | (23.0%) | 182 | (11.2%) | 0.013 |
| Obesity, n (%) | 2 | (15.4%) | 97 | (5.1%) | 0.692 |
| Dyslipidemia, n (%) | 13 | (100%) | 253 | (16.6%) | <0.001 |
| Cerebrovascular disease, n (%) | 1 | (7.7%) | 45 | (2.3%) | <0.001 |
| Chronic liver disease, n (%) | 0 | (0%) | 8 | (2.1%) | 0.005 |
| Chronic kidney disease, n (%) | 1 | (7.7%) | 93 | (3.1%) | 0.3099 |

Laboratory Examination on Admission

| | | | | | |
|---|---|---|---|---|---|
| Platelets count, median (IQR) | 4 | (3.03-7.3) | 209.0 | (161.0-266.8) | 0.289 |
| Neutrophil count, median (IQR) | 1.05 | (0.9-1.48) | 4.3 | (2.9-6.4) | 0.641 |
| Lymphocyte count, median (IQR) | 7.79 | (5.55-12.69) | 20.1 | (12.8-28.1) | 0.554 |
| C-reactive protein, median (IQR) | 10.84 | (7.57-16.14) | 5.6 | (1.3-16.6) | <0.001 |
| Cardiac Troponin, median (IQR) | 415.0 | (311.7-622.4) | 8.9 | (4.5-24.0) | 0.001 |
| Ferritin, median (IQR) | 101 | (65-120.2) | 255.9 | (97.4-663.4) | 0.004 |
| Creatinine, median (IQR) | 313 | (268.5-329) | 74.0 | (59.0-92.0) | 0.319 |
| LDH, median (IQR) | 1.1 | (0.61-2.31) | 237.0 | (185.0-319.0) | 0.127 |
| D-dimer, median (IQR) | 8.1 | (7-11.22) | 0.6 | (0.3-1.3) | 0.569 |
| T. Bilirubin, median (IQR) | 2.15 | (1.58-2.95) | 7.9 | (5.8-11.5) | 0.7953 |
| Lactic Acid, median (IQR) | 7.6 | (5-9.1) | 2.0 | (1.6-2.9) | 0.346 |
| Glucose, median (IQR) | 4 | (3.03-7.3) | 5.6 | (4.8-7.1) | 0.213 |

Statistical Outcomes

| | | | | | |
|---|---|---|---|---|---|
| Follow-up days, median (IQR) | 5.9 | (3-10.7) | 3.4 | (1.4-7.9) | <0.001 |
| ICU admission, n (%) | 1 | (7.7%) | 319 | (21%) | 0.013 |
| Mechanically Ventilated/ECMO, n (%) | 1 | (7.7%) | 102 | (6.7%) | 0.864 |
| Non-invasive positive pressure ventilation, n (%) | 0 | (0%) | 45 | (3.0%) | <0.001 |
| High Flow Oxygen, n (%) | 3 | (23.1%) | 146 | (9.6%) | 0.863 |
| Oxygen Supplementation, n (%) | 9 | (69.2%) | 325 | (21.4%) | 0.054 |

| | | | |
|---|---|---|---|
| | Statins | Non-statins | p |
| Parameters | (N = 363) | (N = 1,169) | value |

Clinical Characteristics on Admission

| | | | | | |
|---|---|---|---|---|---|
| Age, median (IQR) | 66.4 | (59.1-75.7) | 47.2 | (32.0-63.5) | <0.001 |
| Female gender, n (%) | 145 | (39.9%) | 479 | (41.0%) | 0.002 |
| Heart rate, median (IQR), bpm | 83.0 | (73.0-94.0) | 86.0 | (75.0-97.0) | 0.764 |
| SBP, median (IQR), mmHg | 131.0 | (119.0-147.0) | 123.0 | (111.0-137.0) | <0.001 |
| DBP, median (IQR), mmHg | 72.0 | (63.0-83.0) | 73.0 | (65.0-81.0) | 0.577 |
| SpO2, median (IQR) | 95.0 | (93.0-98.0) | 97.0 | (95.0-99.0) | <0.001 |
| Temperature (P.O), median (IQR) | 36.9 | (36.6-37.4) | 36.9 | (36.7-37.4) | 0.303 |
| Smoking, n (%) | 21 | (5.8%) | 30 | (2.6%) | 0.047 |

Comorbidities on Admission

| | | | | | |
|---|---|---|---|---|---|
| Asthma, n (%) | 11 | (3.0%) | 33 | (2.8%) | 0.719 |
| COPD, n (%) | 21 | (5.8%) | 12 | (1.0%) | <0.001 |
| Hypertension, n (%) | 205 | (56.5%) | 197 | (19.8%) | <0.001 |
| Diabetes, n (%) | 175 | (48.2%) | 120 | (10.3%) | <0.001 |
| Coronary heart disease, n (%) | 108 | (29.7%) | 50 | (4.3%) | <0.001 |
| Obesity, n (%) | 38 | (10.5%) | 54 | (4.5%) | 0.005 |
| Dyslipidemia, n (%) | 176 | (48.5%) | 78 | (6.7%) | <0.001 |
| Cerebrovascular disease, n (%) | 31 | (8.5%) | 13 | (1.1%) | <0.001 |
| Chronic liver disease, n (%) | 3 | (0.8%) | 5 | (0.4%) | 0.559 |
| Chronic kidney disease, n (%) | 47 | (12.9%) | 40 | (3.4%) | <0.001 |

Laboratory Examination on Admission

| | | | | | |
|---|---|---|---|---|---|
| Platelet count, median (IQR) | 195.0 | (145.0-256.0) | 213.0 | (163.0-270.0) | <0.001 |
| Neutrophil count, median (IQR) | 4.3 | (3.0-6.2) | 4.3 | (2.9-6.4) | 0.811 |
| Lymphocyte count, median (IQR) | 1.0 | (0.7-1.4) | 1.1 | (0.7-1.6) | 0.104 |
| C-reactive protein, median (IQR) | 9.2 | (2.6-18.7) | 5.4 | (1.4-15.3) | 0.167 |
| Cardiac Troponin, median (IQR) | 10.5 | (5.9-23.5) | 8.0 | (3.9-22.0) | 0.066 |
| Ferritin, median (IQR) | 353.1 | (156.8-741.5) | 249.7 | (81.4-643.5) | 0.050 |
| Creatinine, median (IQR) | 87.0 | (70.0-144.0) | 70.0 | (57.0-85.0) | 0.023 |
| LDH, median (IQR) | 264.5 | (208.0-341.8) | 238.0 | (185.0-311.0) | 0.300 |
| D-dimer, median (IQR) | 0.7 | (0.5-1.4) | 0.6 | (0.3-1.2) | 0.079 |
| T. Bilirubin, median (IQR) | 7.6 | (5.8-10.8) | 8.2 | (5.9-11.7) | 0.010 |
| Lactic Acid, median (IQR) | 2.1 | (1.5-2.9) | 2.1 | (1.6-2.9) | 0.562 |
| Glucose, median (IQR) | 6.4 | (5.3-9.3) | 5.4 | (4.7-6.7) | <0.001 |

Statistical Outcomes

| | | | | | |
|---|---|---|---|---|---|
| Follow-up days, median (IQR) | 5.9 | (3.0-11.3) | 3.1 | (1.5-7.5) | 0.026 |
| ICU admission, n (%) | 102 | (28.1%) | 188 | (16.1%) | 0.001 |
| Mechanically Ventilated/ECMO, n (%) | 43 | (11.8%) | 48 | (4.1%) | <0.001 |
| Non-invasive positive pressure ventilation, n (%) | 18 | (5.0%) | 25 | (2.1%) | 0.0813 |
| High Flow Oxygen, n (%) | 60 | (16.5%) | 84 | (7.2%) | <0.001 |
| Oxygen Supplementation, n (%) | 133 | (36.6%) | 196 | (16.8%) | <0.001 |

| | | | |
|---|---|---|---|
| | Metformin | Non-metformin | p |
| Parameters | (N = 137) | (N = 1,395) | value |

Clinical Characteristics on Admission

| | | | | | |
|---|---|---|---|---|---|
| Age, median (IQR) | 67.0 | (60.3-73.2) | 53.0 | (35.0-67.6) | <0.001 |
| Female gender, n (%) | 59 | (43.1%) | 565 | (40.5%) | 0.362 |
| Heart rate, median (IQR), bpm | 85.0 | (79.9-95.0) | 85.0 | (74.0-96.0) | 0.528 |
| SBP, median (IQR), mmHg | 128.5 | (115.0-145.6) | 125.0 | (113.0-139.0) | 0.031 |
| DBP, median (IQR), mmHg | 71.0 | (62.7-82.0) | 73.5 | (65.0-82.0) | 0.832 |
| SpO2, median (IQR) | 95.0 | (93.0-98.0) | 97.0 | (94.0-99.0) | <0.001 |
| Temperature (P.O), median (IQR) | 37.1 | (36.9-37.5) | 36.9 | (36.7-37.4) | 0.654 |
| Smoking, n (%) | 6 | (4.4%) | 45 | (3.2%) | 0.732 |

Comorbidities on Admission

| | | | | | |
|---|---|---|---|---|---|
| Asthma, n (%) | 7 | (5.1%) | 37 | (2.7%) | 0.301 |
| COPD, n (%) | 3 | (2.2%) | 30 | (2.2%) | 0.817 |
| Hypertension, n (%) | 87 | (63.5%) | 315 | (22.6%) | <0.001 |
| Diabetes, n (%) | 110 | (80.3%) | 185 | (13.3%) | <0.001 |
| Coronary heart disease, n (%) | 37 | (27%) | 121 | (8.7%) | <0.001 |
| Obesity, n (%) | 19 | (13.9) | 72 | (5.2) | 0.011 |
| Dyslipidemia, n (%) | 62 | (45.3%) | 192 | (13.8%) | <0.001 |
| Cerebrovascular disease, n (%) | 12 | (8.8%) | 32 | (2.3%) | 0.015 |
| Chronic liver disease, n (%) | 1 | (0.7%) | 7 | (0.5%) | 0.847 |
| Chronic kidney disease, n (%) | 10 | (7.3%) | 77 | (5.5%) | 0.705 |

Laboratory Examination on Admission

| | | | | | |
|---|---|---|---|---|---|
| Platelets count, median (IQR) | 217.5 | (162.8-278.5) | 204.0 | (158.0-263.2) | 0.27 |
| Neutrophil count, median (IQR) | 4.6 | (3.8-5.9) | 4.2 | (2.9-6.4) | 0.872 |
| Lymphocyte count, median (IQR) | 1.1 | (0.7-1.5) | 1.1 | (0.7-1.5) | 0.257 |
| C-reactive protein, median (IQR) | 11.8 | (3.3-22.7) | 5.9 | (1.4-15.5) | 0.037 |
| Cardiac Troponin, median (IQR) | 7.0 | (4.3-15.5) | 9.3 | (4.7-23.4) | 0.728 |
| Ferritin, median (IQR) | 273.6 | (134.0-574.0) | 272.4 | (104.8-716.2) | 0.576 |
| Creatinine, median (IQR) | 85 | (66.5-117.0) | 74.0 | (59.0-92.0) | 0.009 |
| LDH, median (IQR) | 244.0 | (205.5-341.5) | 243.0 | (190.0-321.0) | 0.798 |
| D-dimer, median (IQR) | 0.61 | (0.46-1.1) | 0.62 | (0.35-1.31) | 0.112 |
| T. Bilirubin, median (IQR) | 7.4 | (5.8-10.8) | 8.1 | (5.9-11.6) | 0.099 |
| Lactic Acid, median (IQR) | 2.4 | (1.7-3.1) | 2.1 | (1.6-2.9) | 0.534 |
| Glucose, median (IQR) | 8.2 | (6.3-12.3) | 5.5 | (4.7-6.8) | <0.001 |

Statistical Outcomes

| | | | | | |
|---|---|---|---|---|---|
| Follow-up days, median (IQR) | 5.0 | (2.2-10.5) | 3.9 | (1.7-8.4) | 0.417 |
| ICU admission, n (%) | 42 | (30.7%) | 248 | (17.8%) | 0.017 |
| Mechanically Ventilated/ECMO, n (%) | 17 | (12.44%) | 74 | (5.3%) | 0.055 |
| Non-invasive positive pressure ventilation, n (%) | 10 | (7.3%) | 33 | (2.4%) | 0.063 |
| High Flow Oxygen, n (%) | 26 | (19%) | 118 | (8.5%) | 0.019 |
| Oxygen Supplementation, n (%) | 52 | (38%) | 277 | (19.9%) | 0.003 |

| | | | |
|---|---|---|---|
| | SGLT2i | Non-SGLT2i | p |
| Parameters | (N = 21) | (N = 1511) | value |

Clinical Characteristics on Admission

| | | | | | |
|---|---|---|---|---|---|
| Age, median (IQR) | 64.1 | (57.8-70.0) | 55.0 | (36.7-69.3) | <0.001 |
| Female gender, n (%) | 8 | (5.1%) | 617 | (40.8%) | 0.214 |
| Heart rate, median (IQR), bpm | 90 | (84.0-99.0) | 85.0 | (74.0-96.0) | 0.151 |
| SBP, median (IQR), mmHg | 126.0 | (120.0-137.0) | 12.0 | (113.0-140.0) | 0.720 |
| DBP, median (IQR), mmHg | 70.5 | (61.0-74.0) | 73.0 | (65.0-82.0) | 0.232 |
| SpO2, median (IQR) | 94.0 | (91.0-96.0) | 96.0 | (94.0-98.0) | 0.018 |
| Temperature (P.O), median (IQR) | 37.3 | (37.0-37.8) | 36.9 | (36.7-37.4) | 0.921 |
| Smoking, n (%) | 0 | (0%) | 51 | (3.4%) | <0.001 |

Comorbidities on Admission

| | | | | | |
|---|---|---|---|---|---|
| Asthma, n (%) | 1 | (4.8%) | 43 | (2.8%) | 0.766 |
| COPD, n (%) | 0 | (0%) | 33 | (2.2%) | <0.001 |
| Hypertension, n (%) | 12 | (57.1%) | 390 | (25.8%) | 0.024 |
| Diabetes, n (%) | 19 | (90.5) | 276 | (18.3) | <0.001 |
| Coronary heart, disease, n (%) | 6 | (28.6%) | 152 | (10.1%) | 0.112 |
| Obesity, n (%) | 1 | (4.8%) | 90 | (6%) | 0.656 |
| Dyslipidemia, n (%) | 11 | (52.4%) | 243 | (16.1%) | 0.007 |
| Cerebrovascular disease, n (%) | 1 | (4.8%) | 43 | (2.8%) | 0.766 |
| Chronic liver disease, n (%) | 0 | (0%) | 8 | (0.5%) | 0.005 |
| Chronic kidney disease, n (%) | 2 | (9.5%) | 85 | (5.6%) | 0.657 |

Laboratory Examination on Admission

| | | | | | |
|---|---|---|---|---|---|
| Platelets count, median (IQR) | 168.5 | (128.8-246.5) | 207.0 | (159.2-265.0) | 0.055 |
| Neutrophil count, median (IQR) | 4.6 | (2.8-6.9) | 4.3 | (2.9-6.2) | 0.878 |
| Lymphocyte count, median (IQR) | 0.5 | (0.7-1.2) | 1.1 | (0.1-1.5) | 0.041 |
| C-reactive protein, median (IQR) | 12.5 | (6.1-24.1) | 6.3 | (1.5-16.9) | 0.229 |
| Cardiac Troponin, median (IQR) | 8.0 | (6.2-11.3) | 8.9 | (4.6-23.4) | 0.001 |
| Ferritin, median (IQR) | 331.0 | (201.4-704.5) | 272.4 | (108.1-692.2) | 0.493 |
| Creatinine, median (IQR) | 106.0 | (77.0-117.2) | 74.0 | (60.0-94.0) | 0.049 |
| LDH, median (IQR) | 257.0 | (195.0-433.0) | 243.5 | (192.2-321.0) | 0.653 |
| D-dimer, median (IQR) | 0.9 | (0.6-1.6) | 0.6 | (0.4-1.3) | 0.882 |
| T. Bilirubin, median (IQR) | 7.8 | (6.6-11.6) | 8.0 | (5.8-11.5) | 0.873 |
| Lactic Acid, median (IQR) | 1.9 | (1.7-3.1) | 2.1 | (1.6-2.9) | 0.966 |
| Glucose, median (IQR) | 8.8 | (8.0-10.0) | 5.6 | (4.8-7.1) | 0.004 |

Statistical Outcomes

| | | | | | |
|---|---|---|---|---|---|
| Follow-up days, median (IQR) | 9.0 | (6.6-15.3) | 4.0 | (1.7-8.5) | 0.08 |
| ICU admission, n (%) | 9 | (42.9%) | 281 | (18.6%) | 0.068 |
| Mechanically Ventilated/ECMO, n (%) | 5 | (23.8%) | 86 | (5.7%) | 0.076 |
| Non-invasive positive pressure ventilation, n (%) | 2 | (9.5%) | 41 | (2.7%) | 0.335 |
| High Flow Oxygen, n (%) | 7 | (33.3%) | 13 | (9.1%) | 0.032 |
| Oxygen Supplementation, n (%) | 11 | (52.4%) | 318 | (21%) | 0.003 |

| | | | |
|---|---|---|---|
| | TZDs | Non-TZDs | p |
| Parameters | (N = 13) | (N = 1519) | value |

Clinical Characteristics on Admission

| | | | | | |
|---|---|---|---|---|---|
| Age, median (IQR) | 64 | (57.6-69.1) | 55.4 | (37.0-69.5) | 0.03 |
| Female gender, n (%) | 9 | (69.2%) | 615 | (40.5%) | 0.115 |
| Heart rate, median (IQR), bpm | 90 | (82-99) | 85.0 | (74.0-96.0) | 0.115 |
| SBP, median (IQR), mmHg | 131 | (121-145.5) | 125.0 | (113.0-139.8) | 0.132 |
| DBP, median (IQR), mmHg | 73 | (66-84) | 73.0 | (65.0-82.0) | 0.492 |
| SpO2, median (IQR) | 95 | (94-98) | 96.0 | (94.0-98.0) | 0.335 |
| Temperature (P.O), median (IQR) | 37.1 | (36.9-37.5) | 36.9 | (36.7-37.4) | 0.572 |
| Smoking, n (%) | 0 | (0%) | 51 | (3.4%) | <0.001 |

Comorbidities on Admission

| | | | | | |
|---|---|---|---|---|---|
| Asthma, n (%) | 0 | (0%) | 44 | (2.9%) | <0.001 |
| COPD, n (%) | 0 | (0%) | 33 | (2.2%) | <0.001 |
| Hypertension, n (%) | 8 | (61.5%) | 394 | (25.9%) | 0.046 |
| Diabetes, n (%) | 11 | (84.6%) | 284 | (18.7%) | <0.001 |
| Coronary heart disease, n (%) | 3 | (23.1%) | 155 | (10.2%) | 0.376 |
| Obesity, n (%) | 2 | (15.4%) | 89 | (5.9%) | 0.428 |
| Dyslipidemia, n (%) | 7 | (53.8%) | 246 | (16.2%) | 0.030 |
| Cerebrovascular disease, n (%) | 2 | (15.4%) | 42 | (2.8%) | 0.266 |
| Chronic liver disease, n (%) | 0 | (0%) | 8 | (0.5%) | 0.005 |
| Chronic kidney disease, n (%) | 1 | (7.7%) | 86 | (5.7%) | 0.889 |

Laboratory Examination on Admission

| | | | | | |
|---|---|---|---|---|---|
| Platelets count, median (IQR) | 216.5 | (172.5-284.2) | 206.0 | (159.0-265.0) | 0.373 |
| Neutrophil count, median (IQR) | 4.5 | (3.85-5.85) | 4.3 | (2.9-6.3) | 0.793 |
| Lymphocyte count, median (IQR) | 1.1 | (1.1-1.45) | 1.1 | (0.7-1.5) | 0.594 |
| C-reactive protein, median (IQR) | 8.64 | (6.32-14.69) | 6.3 | (1.5-17.1) | <0.001 |
| Cardiac Troponin, median (IQR) | 9.02 | (5.79-12.96) | 8.9 | (4.6-23.0) | 0.002 |
| Ferritin, median (IQR) | 297.6 | (131.6-473.1) | 276.0 | (109.5-715.0) | <0.001 |
| Creatinine, median (IQR) | 78 | (66.5-105) | 74.5 | (60.0-94.0) | 0.002 |
| LDH, median (IQR) | 293.5 | (220-383.8) | 243.0 | (192.5-321.5) | 0.41 |
| D-dimer, median (IQR) | 0.74 | (0.37-0.98) | 0.62 | (0.36-1.3) | <0.001 |
| T. Bilirubin, median (IQR) | 7.3 | (6.6-11.65) | 8.0 | (5.8-11.5) | 0.79 |
| Lactic Acid, median (IQR) | 1.8 | (1.4-2.5) | 2.1 | (1.6-2.9) | 0.267 |
| Glucose, median (IQR) | 9.3 | (6.95-15.1) | 5.6 | (4.8-7.2) | 0.011 |

Statistical Outcomes

| | | | | | |
|---|---|---|---|---|---|
| Follow-up days, median (IQR) | 8.5 | (7.5-15) | 4.0 | (1.8-8.7) | 0.789 |
| ICU admission, n (%) | 6 | (46.1%) | 287 | (18.9%) | 0.913 |
| Mechanically Ventilated/ECMO, n (%) | 8 | (61.5%) | 89 | (5.9%) | 0.415 |
| Non-invasive positive pressure ventilation, n (%) | 2 | (15.4%) | 42 | (2.8%) | 0.568 |
| High Flow Oxygen, n (%) | 12 | (92.3%) | 142 | (9.3%) | 0.650 |
| Oxygen Supplementation, n (%) | 6 | (46.2%) | 324 | (21.3%) | 0.292 |

| | | | |
|---|---|---|---|
| | IREi | Non-IREi | p |
| Parameters | (N = 108) | (N = 1424) | value |

Clinical Characteristics on Admission

| | | | | | |
|---|---|---|---|---|---|
| Age, median (IQR) | 67.5 | (57.0-76.0) | 53.7 | (35.5-68.0) | <0.001 |
| Female gender, n (%) | 54 | (50.0%) | 570 | (40%) | 0.483 |
| Heart rate, median (IQR), bpm | 83.2 | (70.0-92.0) | 85.0 | (74.5-96.0) | 0.021 |
| SBP, median (IQR), mmHg | 142.0 | (126.0-150.5) | 124.0 | (112.0-137.0) | <0.001 |
| DBP, median (IQR), mmHg | 78.0 | (67.5-84.5) | 73.0 | (64.0-82.0) | 0.564 |
| SpO2, median (IQR) | 95.0 | (93.0-98.0) | 97.0 | (94.0-98.0) | 0.034 |
| Temperature (P.O), median (IQR) | 37.1 | (36.7-37.7) | 36.9 | (36.7-37.4) | 0.298 |
| Smoking, n (%) | 3 | (2.8%) | 48 | (3.4%) | 0.482 |

Comorbidities on Admission

| | | | | | |
|---|---|---|---|---|---|
| Asthma, n (%) | 2 | (1.9%) | 42 | (2.9%) | 0.251 |
| COPD, n (%) | 6 | (5.6%) | 27 | (1.9%) | 0.143 |
| Hypertension, n (%) | 88 | (81.5%) | 314 | (22.1%) | <0.001 |
| Diabetes, n (%) | 40 | (37.0%) | 255 | (17.9%) | 0.001 |
| Coronary heart disease, n (%) | 30 | (27.8%) | 128 | (9.0%) | <0.001 |
| Obesity, n (%) | 18 | (16.7%) | 73 | (5.1%) | 0.004 |
| Dyslipidemia, n (%) | 45 | (41.7%) | 209 | (14.7%) | <0.001 |
| Cerebrovascular disease, n (%) | 8 | (7.4%) | 36 | (2.5%) | 0.088 |
| Chronic liver disease, n (%) | 1 | (0.9%) | 7 | (0.5%) | 0.716 |
| Chronic kidney disease, n (%) | 15 | (13.9%) | 72 | (5.1%) | 0.022 |

Laboratory Examination on Admission

| | | | | | |
|---|---|---|---|---|---|
| Platelets count, median (IQR) | 194.0 | (156.5-269.5) | 208.0 | (159.0-265.0) | 0.958 |
| Neutrophil count, median (IQR) | 4.3 | (2.9-6.9) | 4.3 | (3.0-6.2) | 0.549 |
| Lymphocyte count, median (IQR) | 1.0 | (0.7-1.3) | 1.1 | (0.7-1.6) | 0.001 |
| C-reactive protein, median (IQR) | 11.6 | (3.1-20.9) | 6.0 | (1.4-16.0) | 0.096 |
| Cardiac Troponin, median (IQR) | 15 | (8.4-40.3) | 8.0 | (4.4-20.6) | 0.848 |
| Ferritin, median (IQR) | 511.6 | (203.6-1106.2) | 263.2 | (100.1-652.9) | 0.121 |
| Creatinine, median (IQR) | 98.0 | (78.8-188.5) | 73.0 | (59.0-91.0) | 0.332 |
| LDH, median (IQR) | 266.5 | (207.2-340.2) | 241.0 | (192.0-321.0) | 0.223 |
| D-dimer, median (IQR) | 0.54 | (0.37-0.58) | 0.62 | (0.36-1.3) | 0.879 |
| T. Bilirubin, median (IQR) | 8.6 | (6.3-11.4) | 7.9 | (5.8-11.5) | 0.264 |
| Lactic Acid, median (IQR) | 2.0 | (1.3-2.9) | 2.1 | (1.6-2.9) | 0.296 |
| Glucose, median (IQR) | 6.4 | (5.0-9.1) | 5.6 | (4.8-7.1) | 0.065 |

Statistical Outcomes

| | | | | | |
|---|---|---|---|---|---|
| Follow-up days, median (IQR) | 6.8 | (3.85-13.0) | 3.8 | (1.7-8.12) | 0.041 |
| ICU days, n (%) | 38 | (35.2%) | 252 | (17.7%) | 0.003 |
| Mechanically Ventilated/ECMO, n (%) | 15 | (13.9%) | 76 | (5.3%) | 0.023 |
| Non-invasive positive pressure ventilation, n (%) | 9 | (8.3%) | 34 | (2.4%) | 0.04 |
| High Flow Oxygen, n (%) | 21 | (19.4%) | 123 | (8.6%) | 0.014 |
| Oxygen Supplementation, n (%) | 39 | (36.1%) | 290 | (20.4) | 0.005 |

TABLE 3
Observational comparison between unique patients visits Hadassah medical center during November
2015-2020 taking metabolic regulators and unique patients in various hospitalization conditions
in patients with COVID-19 taking metabolic regulators. Patients taking Metformin and TZDs
are significantly overrepresented in COVID hospitalizations compared to their portions of
patients. Patients taking any metabolic regulators, but Fibrates, are significantly overrepresented
in COVID ICU admissions and COVID-related deaths compared to their portions of the patient
population in Israel. Fibrates users show a non-significant under-representation across all
COVID terms. Comparisons was done using Fisher's exact test or χ2 test.

| | | | | | | | Adults |
|---|---|---|---|---|---|---|---|
| Israel | Fibrates | Statins | SGLT2i | TZDs | Metformin | IREi | Over 40 |
| Patients Records | 3,249 | 46,030 | 2,057 | 490 | 17,536 | 12,675 | 249,939 |
| 2015-2020 | 1.30% | 18.42% | 0.82% | 0.20% | 7.02% | 5.07% | 100% |
| COVID-19 | 13 | 306 | 16 | 13 | 135 | 72 | 1,531 |
| Hospitalization | 0.85% | 19.99% | 1.05% | 0.85% | 8.82% | 4.70% | 100% |
| IRR | 0.65 | 1.09 | 1.27 | 4.33 | 1.26 | 0.93 | |
| 95% CL | 0.35 | 0.96 | 0.72 | 2.29 | 1.05 | 0.73 | |
| | 1.12 | 1.23 | 2.07 | 7.48 | 1.49 | 1.17 | |
| p-v | 0.12 | 0.19 | 0.34 | 1.41E−08 | 0.01 | 0.53 | |
| | n.s | n.s | n.s | *** | * | n.s | |
| COVID-19 | 1 | 133 | 9 | 3 | 42 | 38 | 312 |
| ICU | 0.32% | 42.63% | 2.88% | 0.96% | 13.46% | 12.18% | 100% |
| IRR | 0.25 | 2.31 | 3.50 | 5.88 | 1.92 | 2.40 | |
| 95% CL | 0.01 | 1.94 | 1.60 | 1.21 | 1.38 | 1.70 | |
| | 1.37 | 2.74 | 6.67 | 17.29 | 2.59 | 3.30 | |
| p-v | 0.13 | 1.11E−16 | 7.85E−05 | 5.51E−04 | 5.57E−05 | 1.47E−07 | |
| | n.s | *** | *** | *** | *** | *** | |
| COVID-19 | 0 | 53 | 3 | 2 | 13 | 15 | 100 |
| Deceased | 0.00% | 53.00% | 3.00% | 2.00% | 13.00% | 15.00% | 100% |
| IRR | 0.00 | 2.88 | 3.65 | 10.20 | 1.85 | 2.96 | |
| 95% CL | N/A | 2.16 | 0.75 | 1.23 | 0.99 | 1.65 | |
| | 2.84 | 3.76 | 10.67 | 37.05 | 3.17 | 4.88 | |
| p-v | 0.25 | 7.43E−11 | 0.02 | 5.68E−05 | 0.03 | 3.97E−05 | |
| | n.s | *** | * | *** | * | *** | |

TABLE 4
Incidence rate ratios and hazard ratios for secondary outcomes and organ failure in the treatment
groups versus non-treatment groups. IR (100-person-day), incidence rate; IRR, incidence rate ratio;
aHR, adjusted hazard ratio; CI, confidence interval; ICU, intensive care unit; IRR p values were
calculated by R package “fmsb”; the significant probability of the result of null-hypothesis
testing. Adjusted HR was calculated based on mixed-effect Cox model with adjustment of age, gender,
clinical characteristics on admission (heart rate, blood pressure, oxidation and temperature), pre-
existing comorbidities (smoking, asthma, COPD, DM, hypertension, diabetes, coronary heart disease,
obesity, dyslipidemia, cerebrovascular disease, chronic liver disease, and chronic kidney disease)
and indicators of disease severity and organ injuries on admission (platelets, neutrophil, lymphocyte
counts, and c-reactive protein, cardiac troponin, ferritin, creatinine, LDH, d-dimer, bilirubin,
lactic acid and glucose levels). aHR p values were calculated from the mixed-effect Cox model.

| | IR | IRR | | p | aHR | | P |
|---|---|---|---|---|---|---|---|
| Fibrates | | | | | | | |
| Invasive mechanical | 0.08 versus 0.38 | 0.21 | (0.01-0.98) | 0.015 | 0.56 | (0.26-1.2) | 0.014 |
| ventilation | | | | | | | |
| ICU admission | 0.08 versus 0.42 | 0.19 | (0.01-0.82) | 0.009 | 0.39 | (0.094-1.6) | 0.019 |
| Septic shock | 0 versus 0.23 | 0 | (−1.38) | 0.096 | 1.40E−08 | (0-Inf) | — |
| Acute liver injury | 0 versus 0.19 | 0 | (−1.57) | 0.11 | 3.70E−08 | (0-Inf) | — |
| Acute kidney injury | 0.08 versus 0.39 | 0.2 | (0.01-1.15) | 0.089 | 0.33 | (0.045-1.4) | 0.027 |
| Acute cardiac injury | 0 versus 0.42 | 0 | (−0.71) | 0.021 | 3.80E−08 | (0-Inf) | — |
| Statins | | | | | | | |
| Invasive mechanical | 0.15 versus 0.12 | 1.3 | (0.91-1.88) | 0.15 | 1.2 | (0.56-2.6) | 0.53 |
| ventilation | | | | | | | |
| ICU admission | 0.33 versus 0.38 | 0.86 | (0.69-1.06) | 0.21 | 0.87 | (0.55-1.4) | 0.53 |
| Septic shock | 0.13 versus 0.18 | 0.73 | (0.36-1.54) | 0.41 | 0.6 | (0.19-1.9) | 0.76 |
| Acute liver injury | 0.16 versus 0.22 | 0.74 | (0.47-1.13) | 0.18 | 0.68 | (0.22-2.1) | 0.5 |
| Acme kidney injury | 0.25 versus 0.37 | 0.68 | (0.43-1.07) | 0.13 | 0.63 | (0.36-1.1) | 0.007 |
| Acute cardiac injury | 0.23 versus 0.29 | 0.8 | (0.57-1.12) | 0.23 | 0.72 | (0.33-1.6) | 0.42 |
| Metformin | | | | | | | |
| Invasive mechanical | 0.15 versus 0.12 | 1.3 | (0.70-2.32) | 0.39 | 1.3 | (0.67-2.5) | 0.45 |
| ventilation | | | | | | | |
| ICU admission | 0.37 versus 0.31 | 1.2 | (0.81-1.71) | 0.41 | 1.1 | (0.52-2.2) | 0.85 |
| Septic shock | 0.16 versus 0.12 | 1.3 | (0.65-2.50) | 0.43 | 1.6 | (0.55-4.9) | 0.37 |
| Acute liver injury | 0.05 versus 0.11 | 0.43 | (0.80-1.48) | 0.17 | 0.76 | (0.22-2.7) | 0.67 |
| Acute kidney injury | 0.18 versus 0.37 | 0.47 | (0.32-0.89) | 0.03 | 0.48 | (0.26-0.89) | 0.02 |
| Acute cardiac injury | 0.25 versus 0.36 | 0.69 | (0.41-1.14) | 0.19 | 1.1 | (0.62-2.1) | 0.69 |

| | IR | IRR | | p | aHR | | p |
|---|---|---|---|---|---|---|---|
| SGLT2 Inhibitors | | | | | | | |
| Invasive mechanical | 0.2 versus 0.21 | 1.06 | (0.28-2.91) | 0.9 | 1.2 | (0.28-5) | 0.83 |
| ventilation | | | | | | | |
| ICU admission | 0.27 versus 0.34 | 0.8 | (0.29-1.82) | 0.65 | 0.93 | (0.4-2.1) | 0.86 |
| Septic shock | 0.22 versus 0.13 | 1.76 | (0.61-4.19) | 0.23 | 1.6 | (0.36-7.3) | 0.52 |
| Acute liver injury | 0.15 versus 0.08 | 1.84 | (0.20-7.74) | 0.44 | 2.6 | (0.58-12) | 0.21 |
| Acute kidney injury | 0.36 versus 0.31 | 1.15 | (0.36-2.81) | 0.8 | 0.9 | (0.36-2.2) | 0.82 |
| Acute cardiac injury | 0.09 versus 0.34 | 0.26 | (0.01-1.55) | 0.18 | 0.2 | (0.03-1.5) | 0.11 |
| Thiazolidinediones | | | | | | | |
| Invasive mechanical | 0.62 versus 0.26 | 2.4 | (0.92-4.96) | 0.04 | 1.6 | (0.64-4.3) | 0.3 |
| ventilation | | | | | | | |
| ICU admission | 0.46 versus 0.34 | 1.35 | (0.74-2.62) | 0.07 | 1.4 | (0.95-2.1) | 0.09 |
| Septic shock | 0.42 versus 0.14 | 2.92 | (0.89-7.52) | 0.04 | 1.3 | (0.17-10) | 0.78 |
| Acute liver injury | 0.1 versus 0.09 | 1.14 | (0.03-7.25) | 0.9 | 1.6 | (0.21-12) | 0.64 |
| Acute kidney injury | 0.43 versus 0.28 | 1.51 | (0.30-4.68) | 0.56 | 2.8 | (0.63-13) | 0.17 |
| Acute cardiac injury | 0.38 versus 0.32 | 1.17 | (0.23-3.66) | 0.82 | 1.2 | (0.59-2.3) | 0.65 |
| IRE1α Inhibiting Drugs | | | | | | | |
| Invasive mechanical | 0.14 versus 0.15 | 0.93 | (0.44-1.90) | 0.9 | 0.91 | (0.45-1.8) | 0.8 |
| ventilation | | | | | | | |
| ICU admission | 0.33 versus 0.39 | 0.85 | (0.58-1.24) | 0.46 | 0.94 | (0.64-1.4) | 0.78 |
| Septic shock | 0.08 versus 0.17 | 0.48 | (0.17-1.15) | 0.09 | 0.45 | (0.1-1.9) | 0.17 |
| Acute liver injury | 0.04 versus 0.08 | 1.52 | (0.06-2.15) | 0.38 | 0.45 | (0.1-1.9) | 0.28 |
| Acute kidney injury | 0.23 versus 0.34 | 0.68 | (0.39-1.12) | 0.16 | 0.7 | (0.43-1.1) | 0.16 |
| Acute cardiac injury | 0.32 versus 0.34 | 0.95 | (0.55-1.54) | 0.85 | 0.79 | (0.4-1.6) | 0.49 |

TABLE 5
Characteristics of metabolic regulators users after PSM (methods). SBP, systolic blood pressure;
DBP, diastolic blood pressure; COPD, chronic obstructive pulmonary disease; SpO2, oxygen saturation;
IQR, interquartile range; SD, standardized difference. The groups were matched based on variables
including adjustment of age, gender, clinical characteristics on admission (heart rate, blood
pressure, oxidation, and temperature), pre-existing comorbidities (smoking, asthma, COPD, DM,
hypertension, diabetes, coronary heart disease, obesity, dyslipidemia, cerebrovascular disease,
chronic liver disease, and chronic kidney disease) and indicators of disease severity and organ
injuries on admission (platelets, neutrophil, lymphocyte counts, and c-reactive protein, cardiac
troponin, ferritin, creatinine, LDH, d-dimer, bilirubin, lactic acid, and glucose levels). P
values were calculated by Mann-Whitney U test for non-normally distributed continuous variables
and Fisher's exact test or χ2 test for categorical variables.

| | | | |
|---|---|---|---|
| | Fibrates | Non-fibrates | p |
| Parameters | (N = 13) | (N = 130) | Value |

Clinical Characteristics on Admission

| | | | | | |
|---|---|---|---|---|---|
| Age, median (IQR) | 69.7 | (57.45-74.4) | 64.5 | (58.15-75.5) | 0.901 |
| Female gender, n (%) | 6 | (46.2%) | 59 | (45.4%) | 0.548 |
| Heart rate, median (IQR), bpm | 81 | (73.5-90.5) | 85 | (75-94) | 0.385 |
| SBP, median (IQR), mmHg | 139.5 | (124.5-146) | 131 | (119-147) | 0.091 |
| DBP, median (IQR), mmHg | 60 | (58-83.5) | 73 | (64-82) | 0.146 |
| SpO2, median (IQR) | 94 | (93-95.5) | 95 | (93-98) | 0.38 |
| Temperature (P.O), median (IQR) | 37 | (36.65-37.3) | 36.9 | (36.6-37.4) | 0.203 |
| Smoking, n (%) | 0 | (0%) | 9 | (6.9%) | 0.597 |

Comorbidities on Admission

| | | | | | |
|---|---|---|---|---|---|
| Asthma, n (%) | 0 | (0%) | 7 | (5.4%) | 1 |
| COPD, n (%) | 0 | (0%) | 6 | (4.6%) | 1 |
| Hypertension, n (%) | 10 | (76.9%) | 88 | (67.7%) | 0.812 |
| Diabetes, n (%) | 8 | (61.5%) | 70 | (53.8%) | 0.785 |
| Coronary heart disease, n (%) | 3 | (23.0%) | 39 | (23.8%) | 0.732 |
| Obesity, n (%) | 2 | (15.4%) | 20 | (15.4%) | 1 |
| Dyslipidemia, n (%) | 13 | (100%) | 130 | (100%) | 1 |
| Cerebrovascular disease, n (%) | 1 | (7.7%) | 9 | (6.9%) | 0.895 |
| Chronic liver disease, n (%) | 0 | (0%) | 3 | (2.3%) | 1 |
| Chronic kidney disease, n (%) | 1 | (7.7%) | 21 | (16.2%) | 0.671 |

Laboratory Examination on Admission

| | | | | | |
|---|---|---|---|---|---|
| Platelet count, median (IQR) | 227 | (196-259.2) | 210 | (159.5-263.5) | 0.148 |
| Neutrophil count, median (IQR) | 4 | (3.03-7.3) | 4.5 | (3.03-6.2) | 0.726 |
| Lymphocyte count, median (IQR) | 1.05 | (0.9-1.48) | 1 | (0.7-1.3) | 0.107 |
| C-reactive protein, median (IQR) | 7.79 | (5.55-12.69) | 7.51 | (2.71-14.14) | 0.605 |
| Cardiac Troponin, median (IQR) | 10.84 | (7.57-16.14) | 11.06 | (5.78-21.71) | 0.868 |
| Ferritin, median (IQR) | 415.0 | (311.7-622.4) | 311.4 | (161.8-729.0) | 0.08 |
| Creatinine, median (IQR) | 101 | (65-120.2) | 79.5 | (66-108.5) | 0.916 |
| LDH, median (IQR) | 313 | (268.5-329) | 275 | (218.2-391.5) | 0.824 |
| D-dimer, median (IQR) | 1.1 | (0.61-2.31) | 0.63 | (0.46-1.73) | 0.943 |
| T. Bilirubin, median (IQR) | 8.1 | (7-11.22) | 7.9 | (6-10.7) | 0.736 |
| Lactic Acid, median (IQR) | 2.15 | (1.58-2.95) | 2.25 | (1.63-2.9) | 0.276 |
| Glucose, median (IQR) | 7.6 | (5-9.1) | 6.5 | (5.4-9.23) | 1 |

Statistical Outcomes

| | | | | | |
|---|---|---|---|---|---|
| Follow-up days, median (IQR) | 5.9 | (3-10.7) | 9.5 | (4.95-15.5) | 0.259 |
| ICU Admission, n (%) | 1 | (7.7%) | 55 | (42.3%) | 0.015 |
| Mechanically Ventilated/ECMO, n (%) | 1 | (7.7%) | 49 | (37.7%) | 0.009 |
| Non-invasive positive pressure ventilation, n (%) | 0 | (0%) | 6 | (4.6%) | 0.357 |
| High Flow Oxygen, n (%) | 3 | (23.1%) | 24 | (18.5%) | 0.107 |
| Oxygen Supplementation, n (%) | 9 | (69.2%) | 54 | (41.5%) | 0.603 |

| | | | |
|---|---|---|---|
| | Statins | Non-statins | p |
| Parameters | (N = 360) | (N = 360) | Value |

Clinical Characteristics on Admission

| | | | | | |
|---|---|---|---|---|---|
| Age, median (IQR) | 65.7 | (59.02-76) | 64.8 | (55.7-80) | 0.171 |
| Female gender, n (%) | 148 | (41.1%) | 202 | (56.1%) | 0.266 |
| Heart rate, median (IQR), bpm | 82 | (73-94) | 85 | (77-90.5) | 0.945 |
| SBP, median (IQR), mmHg | 130 | (119-145) | 131 | (119-145.1) | 0.381 |
| DBP, median (IQR), mmHg | 72 | (63-83) | 73.5 | (65.75-80) | 0.865 |
| SpO2, median (IQR) | 95 | (93-98) | 95 | (93-98) | 0.263 |
| Temperature (P.O), median (IQR) | 36.9 | (36.6-37.4) | 36.95 | (36.7-37.4) | 0.758 |
| Smoking, n (%) | 18 | (5%) | 13 | (3.6%) | 0.622 |

Comorbidities on Admission

| | | | | | |
|---|---|---|---|---|---|
| Asthma, n (%) | 13 | (3.6%) | 6 | (1.7%) | 0.75 |
| COPD, n (%) | 23 | (6.4%) | 6 | (1.7%) | 0.442 |
| Hypertension, n (%) | 196 | (54.4%) | 240 | (66.7%) | 0.209 |
| Diabetes, n (%) | 175 | (48.6%) | 171 | (47.5%) | 1 |
| Coronary heart disease, n (%) | 87 | (24.2%) | 69 | (12.2%) | 0.218 |
| Obesity, n (%) | 37 | (10.3%) | 63 | (17.5%) | 0.72 |
| Dyslipidemia, n (%) | 153 | (42.5%) | 150 | (41.7%) | 0.941 |
| Cerebrovascular disease, n (%) | 26 | (7.2%) | 13 | (3.6%) | 0.534 |
| Chronic liver disease, n (%) | 4 | (1.1%) | 6 | (1.7%) | 0.303 |
| Chronic kidney disease, n (%) | 55 | (15.3%) | 38 | (10.6%) | 0.868 |

Laboratory Examination on Admission

| | | | | | |
|---|---|---|---|---|---|
| Platelet count, median (IQR) | 196 | (150-259) | 208.5 | (160-262.5) | 0.109 |
| Neutrophil count, median (IQR) | 4.2 | (2.9-6) | 4.6 | (3-7.55) | 0.025 |
| Lymphocyte count, median (IQR) | 1 | (0.7-1.4) | 0.9 | (0.6-1.25) | 0.207 |
| C-reactive protein, median (IQR) | 7.6 | (2.23-15) | 8.73 | (3.31-14.34) | 0.244 |
| Cardiac Troponin, median (IQR) | 10.56 | (6.1-30.02) | 13.25 | (7.89-23.54) | 0.029 |
| Ferritin, median (IQR) | 320.0 | (153.1-711.9) | 322.8 | (192.1-643.5) | 0.472 |
| Creatinine, median (IQR) | 87 | (70-144) | 76 | (62-101) | 0.126 |
| LDH, median (IQR) | 264.5 | (208-341.8) | 287 | (232-407) | 0.204 |
| D-dimer, median (IQR) | 0.7 | (0.45-1.38) | 0.74 | (0.53-1.41) | 0.303 |
| T. Bilirubin, median (IQR) | 7.6 | (5.8-10.8) | 8.1 | (6.3-11.1) | 0.533 |
| Lactic Acid, median (IQR) | 2.15 | (1.5-2.9) | 2.5 | (2-3.3) | 0.206 |
| Glucose, median (IQR) | 6.5 | (5.3-9.3) | 6.35 | (5.1-7.83) | 0.891 |

Statistical Outcomes

| | | | | | |
|---|---|---|---|---|---|
| Follow-up days, median (IQR) | 6 | (3-11.4) | 6.7 | (2.6-11.4) | 0.55 |
| ICU Admission, n (%) | 73 | (20.3%) | 82 | (22.8%) | 0.25 |
| Mechanically Ventilated/ECMO, n (%) | 46 | (12.8%) | 66 | (18.3%) | 0.632 |
| Non-invasive positive pressure ventilation, n (%) | 7 | (1.9%) | 19 | (5.3%) | 0.613 |
| High Flow Oxygen, n (%) | 61 | (16.9%) | 95 | (26.4%) | 0.769 |
| Oxygen Supplementation, n (%) | 165 | (45.8%) | 180 | (50%) | 0.659 |

| | | | |
|---|---|---|---|
| | Metformin | Non-metformin | p |
| Parameters | (N = 135) | (N = 405) | Value |

Clinical Characteristics on Admission

| | | | | | |
|---|---|---|---|---|---|
| Age, median (IQR) | 63.7 | (59.3-73.1) | 65.3 | (58.15-73.7) | 0.883 |
| Female gender, n (%) | 62 | (45.9%) | 189 | (46.7%) | 0.92 |
| Heart rate, median (IQR), bpm | 86.5 | (80-97) | 82 | (73-94.75) | 0.275 |
| SBP, median (IQR), mmHg | 127 | (116.8-141.2) | 132 | (116.5-146.5) | 0.583 |
| DBP, median (IQR), mmHg | 73 | (65-83) | 70 | (61-80) | 0.363 |
| SpO2, median (IQR) | 95 | (93-97) | 95 | (93-98) | 0.9 |
| Temperature (P.O), median (IQR) | 37.1 | (36.88-37.5) | 36.9 | (36.7-37.2) | 0.004 |
| Smoking, n (%) | 5 | (3.7%) | 17 | (4.2%) | 0.477 |

Comorbidities on Admission

| | | | | | |
|---|---|---|---|---|---|
| Asthma, n (%) | 9 | (6.7%) | 17 | (4.2%) | 0.402 |
| COPD, n (%) | 3 | (2.2%) | 20 | (4.9%) | 0.373 |
| Hypertension, n (%) | 87 | (64.4%) | 248 | (61.2%) | 1 |
| Diabetes, n (%) | 102 | (75.6%) | 405 | (100%) | 0.221 |
| Coronary heart disease, n (%) | 29 | (21.5%) | 114 | (19.8%) | 0.62 |
| Obesity, n (%) | 18 | (13.3%) | 57 | (14.1%) | 1 |
| Dyslipidemia, n (%) | 55 | (40.7%) | 159 | (39.3%) | 0.54 |
| Cerebrovascular disease, n (%) | 10 | (7.4%) | 17 | (4.2%) | 0.682 |
| Chronic liver disease, n (%) | 1 | (0.7%) | 5 | (1.2%) | 1 |
| Chronic kidney disease, n (%) | 13 | (9.6%) | 77 | (19%) | 0.005 |

Laboratory Examination on Admission

| | | | | | |
|---|---|---|---|---|---|
| Platelet count, median (IQR) | 220 | (179-279) | 208 | (161-266) | 0.28 |
| Neutrophil count, median (IQR) | 4.4 | (3.5-5.7) | 4.6 | (3.1-6.9) | 0.52 |
| Lymphocyte count, median (IQR) | 1 | (0.7-1.6) | 1 | (0.7-1.3) | 0.132 |
| C-reactive protein, median (IQR) | 9.35 | (2.86-17.24) | 9.21 | (3.73-16.86) | 0.777 |
| Cardiac Troponin, median (IQR) | 6.78 | (5.3-13.5) | 15.56 | (7.16-44.47) | 0 |
| Ferritin, median (IQR) | 265.6 | (153.3-492.4) | 441 | (178.8-936.8) | 0.035 |
| Creatinine, median (IQR) | 85 | (66.5-117) | 86 | (69-185.5) | 0.023 |
| LDH, median (IQR) | 244 | (205.5-341.5) | 273 | (217-355.2) | 0.061 |
| D-dimer, median (IQR) | 0.6 | (0.48-1.06) | 0.86 | (0.49-1.84) | 0.02 |
| T. Bilirubin, median (IQR) | 7.6 | (5.83-10.78) | 7.4 | (5.6-10) | 0.125 |
| Lactic Acid, median (IQR) | 2.4 | (1.7-3.15) | 2.3 | (1.5-3) | 0.769 |
| Glucose, median (IQR) | 8.2 | (6.25-11.15) | 8.5 | (6.25-11.5) | 0.385 |

Statistical Outcomes

| | | | | | |
|---|---|---|---|---|---|
| Follow-up days, median (IQR) | 4.8 | (2.1-9.7) | 7 | (3.5-14.55) | 0.018 |
| ICU Admission, n (%) | 22 | (16.3%) | 107 | (26.4%) | 0.821 |
| Mechanically Ventilated/ECMO, n (%) | 14 | (10.4%) | 63 | (15.6%) | 0.747 |
| Non-invasive positive pressure ventilation, n (%) | 3 | (2.2%) | 28 | (6.9%) | 1 |
| High Flow Oxygen, n (%) | 21 | (15.6%) | 84 | (20.7%) | 0.775 |
| Oxygen Supplementation, n (%) | 57 | (42.2%) | 199 | (49.1%) | 0.441 |

| | | | |
|---|---|---|---|
| | SGLT2i | Non-SGLT2i | p |
| Parameters | (N = 16) | (N = 160) | Value |

Clinical Characteristics on Admission

| | | | | | |
|---|---|---|---|---|---|
| Age, median (IQR) | 63.7 | (58.92-67.6) | 64.4 | (58.45-73.7) | 0.274 |
| Female gender, n (%) | 6 | (37.5%) | 75 | (46.9%) | 0.672 |
| Heart rate, median (IQR), bpm | 93.5 | (84.25-101.75) | 83 | (75-95) | 0.028 |
| SBP, median (IQR), mmHg | 124 | (119.5-134.4) | 131 | (117.2-145.4) | 0.415 |
| DBP, median (IQR), mmHg | 72 | (64.5-75.5) | 71 | (61-81.75) | 0.373 |
| SpO2, median (IQR) | 93.5 | (91-94.25) | 95 | (93-98) | 0.34 |
| Temperature (P.O), median (IQR) | 37.3 | (36.98-37.73) | 36.95 | (36.7-37.3) | 0.06 |
| Smoking, n (%) | 0 | (0%) | 6 | (3.8%) | 0.613 |

Comorbidities on Admission

| | | | | | |
|---|---|---|---|---|---|
| Asthma, n (%) | 1 | (6.3%) | 8 | (5%) | 0.549 |
| COPD, n (%) | 0 | (0%) | 6 | (3.8%) | 1 |
| Hypertension, n (%) | 11 | (68.8%) | 104 | (65%) | 0.717 |
| Diabetes, n (%) | 14 | (87.5%) | 160 | (100%) | 0.87 |
| Coronary heart disease, n (%) | 5 | (31.3%) | 44 | (20%) | 0.81 |
| Obesity, n (%) | 1 | (6.3%) | 25 | (15.6%) | 0.333 |
| Dyslipidemia, n (%) | 8 | (50%) | 66 | (41.3%) | 0.69 |
| Cerebrovascular disease, n (%) | 1 | (6.3%) | 9 | (5.6%) | 1 |
| Chronic liver disease, n (%) | 0 | (0%) | 2 | (1.3%) | 1 |
| Chronic kidney disease, n (%) | 2 | (12.5%) | 27 | (16.9%) | 0.752 |

Laboratory Examination on Admission

| | | | | | |
|---|---|---|---|---|---|
| Platelet count, median (IQR) | 193 | (154-254) | 219 | (169-272) | 0.26 |
| Neutrophil count, median (IQR) | 4.25 | (2.73-6.35) | 4.6 | (3.3-6.3) | 0.839 |
| Lymphocyte count, median (IQR) | 1.1 | (0.7-1.4) | 1 | (0.7-1.3) | 0.56 |
| C-reactive protein, median (IQR) | 10.22 | (5.95-19.2) | 9.46 | (2.99-16.92) | 0.332 |
| Cardiac Troponin, median (IQR) | 10.86 | (6.86-15.97) | 13.52 | (6.25-34.99) | 0.198 |
| Ferritin, median (IQR) | 202.5 | (198.3-657.9) | 336.4 | (159.6-751.5) | 0.926 |
| Creatinine, median (IQR) | 106 | (77-117.2) | 85 | (68.25-149.25) | 0.807 |
| LDH, median (IQR) | 257 | (195-433) | 273 | (216.5-354) | 0.78 |
| D-dimer, median (IQR) | 0.76 | (0.56-1) | 0.73 | (0.48-1.49) | 0.625 |
| T. Bilirubin, median (IQR) | 7.8 | (6.6-11.6) | 7.3 | (5.58-9.23) | 0.198 |
| Lactic Acid, median (IQR) | 1.9 | (1.7-3.1) | 2.3 | (1.6-3.1) | 0.834 |
| Glucose, median (IQR) | 8.9 | (8.2-9.9) | 8.1 | (6.28-11.25) | 0.655 |

Statistical Outcomes

| | | | | | |
|---|---|---|---|---|---|
| Follow-up days, median (IQR) | 9.85 | (7.13-15.23) | 6.3 | (2.85-13.4) | 0.029 |
| ICU Admission, n (%) | 6 | (37.5%) | 38 | (23.8%) | 0.52 |
| Mechanically Ventilated/ECMO, n (%) | 4 | (25%) | 22 | (13.8%) | 0.348 |
| Non-invasive positive pressure ventilation, n (%) | 0 | (0%) | 8 | (5%) | 0.667 |
| High Flow Oxygen, n (%) | 7 | (43.8%) | 29 | (18.1%) | 0.164 |
| Oxygen Supplementation, n (%) | 13 | (81.3%) | 71 | (44.4%) | 0.293 |

| | | | |
|---|---|---|---|
| | TZDs | Non-TZDs | p |
| Parameters | (N = 13) | (N = 130) | Value |

Clinical Characteristics on Admission

| | | | | | |
|---|---|---|---|---|---|
| Age, median (IQR) | 64 | (57.6-69.1) | 65.7 | (59.08-74.58) | 0.257 |
| Female gender, n (%) | 9 | (69.2%) | 56 | (43.1%) | 0.225 |
| Heart rate, median (IQR), bpm | 90 | (82-99) | 84 | (75-94) | 0.559 |
| SBP, median (IQR), mmHg | 131 | (121-145.5) | 132 | (117.5-148) | 0.45 |
| DBP, median (IQR), mmHg | 73 | (66-84) | 71 | (60.5-80.25) | 0.734 |
| SpO2, median (IQR) | 95 | (94-98) | 95 | (93-98) | 1 |
| Temperature (P.O), median (IQR) | 37.1 | (36.9-37.5) | 37 | (36.7-37.4) | 0.725 |
| Smoking, n (%) | 0 | (0%) | 7 | (5.4%) | 1 |

Comorbidities on Admission

| | | | | | |
|---|---|---|---|---|---|
| Asthma, n (%) | 0 | (0%) | 5 | (3.8%) | 0.616 |
| COPD, n (%) | 0 | (0%) | 5 | (3.8%) | 0.616 |
| Hypertension, n (%) | 8 | (61.5%) | 86 | (66.2%) | 0.823 |
| Diabetes, n (%) | 11 | (84.6%) | 110 | (84.6%) | 1 |
| Coronary heart disease, n (%) | 3 | (23.1%) | 40 | (22.3%) | 0.753 |
| Obesity, n (%) | 2 | (15.4%) | 19 | (14.6%) | 0.427 |
| Dyslipidemia, n (%) | 7 | (53.8%) | 65 | (50.0%) | 0.929 |
| Cerebrovascular disease, n (%) | 2 | (15.4%) | 18 | (13.8%) | 0.831 |
| Chronic liver disease, n (%) | 0 | (0%) | 1 | (0.8%) | 1 |
| Chronic kidney disease, n (%) | 1 | (7.7%) | 20 | (15.4%) | 1 |

Laboratory Examination on Admission

| | | | | | |
|---|---|---|---|---|---|
| Platelet count, median (IQR) | 216.5 | (172.5-284.2) | 217 | (160.5-272) | 0.556 |
| Neutrophil count, median (IQR) | 4.5 | (3.85-5.85) | 4.7 | (3.3-6.7) | 0.771 |
| Lymphocyte count, median (IQR) | 1.1 | (1.1-1.45) | 1 | (0.7-1.4) | 0.052 |
| C-reactive protein, median (IQR) | 8.64 | (6.32-14.69) | 10.89 | (3.73-21.11) | 0.246 |
| Cardiac Troponin, median (IQR) | 9.02 | (5.79-12.96) | 11.36 | (6-25.25) | 0.553 |
| Ferritin, median (IQR) | 297.6 | (131.6-473.1) | 357.1 | (167.1-833.9) | 0.291 |
| Creatinine, median (IQR) | 78 | (66.5-105) | 85 | (69-150) | 0.671 |
| LDH, median (IQR) | 293.5 | (220-383.8) | 265 | (215-354) | 0.987 |
| D-dimer, median (IQR) | 0.74 | (0.37-0.98) | 0.85 | (0.48-1.69) | 0.16 |
| T. Bilirubin, median (IQR) | 7.3 | (6.6-11.65) | 7.3 | (5.55-9.8) | 0.456 |
| Lactic Acid, median (IQR) | 1.8 | (1.4-2.5) | 2.3 | (1.63-3.18) | 0.906 |
| Glucose, median (IQR) | 9.3 | (6.95-15.1) | 8.2 | (6.1-11.42) | 0.42 |

Statistical Outcomes

| | | | | | |
|---|---|---|---|---|---|
| Follow-up days, median (IQR) | 8.5 | (7.5-15) | 6 | (3-12.3) | 0.066 |
| ICU Admission, n (%) | 6 | (46.1%) | 44 | (33.8%) | 0.073 |
| Mechanically Ventilated/ECMO, n (%) | 8 | (61.5%) | 34 | (26.2%) | 0.031 |
| Non-invasive positive pressure ventilation, n (%) | 2 | (15.4%) | 18 | (13.8%) | 0.246 |
| High Flow Oxygen, n (%) | 12 | (92.3%) | 64 | (49.2%) | 0.078 |
| Oxygen Supplementation, n (%) | 6 | (46.2%) | 46 | (35.4%) | 0.479 |

| | | | |
|---|---|---|---|
| | IREi | Non-IREi | p |
| Parameters | (N = 72) | (N = 360) | Value |

Clinical Characteristics on Admission

| | | | | | |
|---|---|---|---|---|---|
| Age, median (IQR) | 67.9 | (57.6-76.0) | 64.7 | (58.5-74.08) | 0.478 |
| Female gender, n (%) | 50 | (69.4%) | 163 | (45.3%) | 0.362 |
| Heart rate, median (IQR), bpm | 95 | (82-99) | 84 | (75-95) | 0.26 |
| SBP, median (IQR), mmHg | 131 | (121-145.5) | 130 | (116-145) | 1 |
| DBP, median (IQR), mmHg | 73 | (66-84) | 70.5 | (61-81) | 0.402 |
| SpO2, median (IQR) | 95 | (94-98) | 95 | (93-98) | 0.499 |
| Temperature (P.O), median (IQR) | 37.1 | (36.9-37.5) | 37 | (36.7-37.4) | 0.337 |
| Smoking, n (%) | 0 | (0%) | 14 | (3.9%) | 0.42 |

Comorbidities on Admission

| | | | | | |
|---|---|---|---|---|---|
| Asthma, n (%) | 0 | (0%) | 19 | (5.3%) | 0.367 |
| COPD, n (%) | 0 | (0%) | 14 | (3.9%) | 0.602 |
| Hypertension, n (%) | 44 | (61.1%) | 236 | (65.6%) | 0.139 |
| Diabetes, n (%) | 61 | (84.7%) | 288 | (80%) | 0.312 |
| Coronary heart disease, n (%) | 17 | (23.6%) | 103 | (20.8%) | 0.806 |
| Obesity, n (%) | 11 | (15.3%) | 53 | (14.7%) | 0.531 |
| Dyslipidemia, n (%) | 28 | (38.8%) | 147 | (40.8%) | 0.917 |
| Cerebrovascular disease, n (%) | 4 | (5.6%) | 19 | (5.3%) | 1 |
| Chronic liver disease, n (%) | 0 | (0%) | 5 | (1.4%) | 1 |
| Chronic kidney disease, n (%) | 12 | (16.7%) | 61 | (16.9%) | 0.875 |

Laboratory Examination on Admission

| | | | | | |
|---|---|---|---|---|---|
| Platelets count, median (IQR) | 216.5 | (172.5-284.2) | 218.5 | (167.5-271.5) | 0.336 |
| Neutrophil count, median (IQR) | 4.5 | (3.85-5.85) | 4.6 | (3.1-6.35) | 0.539 |
| Lymphocyte count, median (IQR) | 1 | (0.7-1.3) | 1.3 | (1.1-1.48) | 0.202 |
| C-reactive protein, median (IQR) | 8.52 | (2.72-17.65) | 9.45 | (3.08-17.29) | 0.734 |
| Cardiac Troponin, median (IQR) | 13.66 | (6.93-41.68) | 13.01 | (6.51-31.18) | 0.198 |
| Ferritin, median (IQR) | 450.2 | (131.6-473.1) | 353.4 | (172.3-786.4) | 0.533 |
| Creatinine, median (IQR) | 78 | (66.5-105) | 80 | (69-140) | 0.165 |
| LDH, median (IQR) | 293.5 | (220-383.8) | 275 | (215-354) | 0.835 |
| D-dimer, median (IQR) | 0.74 | (0.37-1.28) | 0.84 | (0.48-1.49) | 0.49 |
| T. Bilirubin, median (IQR) | 7.3 | (6.6-11.65) | 7.3 | (5.55-9.8) | 0.018 |
| Lactic Acid, median (IQR) | 1.8 | (1.4-2.5) | 2.3 | (1.63-3.18) | 0.212 |
| Glucose, median (IQR) | 10.3 | (6.95-15.1) | 8.2 | (6.23-11.18) | 0 |

Statistical Outcomes

| | | | | | |
|---|---|---|---|---|---|
| Follow-up days, median (IQR) | 4.5 | (3.5-11) | 6.55 | (2.83-13.45) | 0.219 |
| ICU Admission, n (%) | 17 | (23.6%) | 88 | (24.4%) | 0.911 |
| Mechanically Ventilated/ECMO, n (%) | 14 | (19.4%) | 52 | (14.4%) | 1 |
| Non-invasive positive pressure ventilation, n (%) | 7 | (9.7%) | 17 | (4.7%) | 0.835 |
| High Flow Oxygen, n (%) | 14 | (19.4%) | 70 | (19.4%) | 0.774 |
| Oxygen Supplementation, n (%) | 36 | (50%) | 166 | (46.1%) | 0.824 |

### Example 5: Metabolic Regulators Effect on COVID-19 Severity and Progression

In order to investigate the influence of metabolic regulators on COVID-19 severity and progression, a propensity score matching (PSM) model was used to rule out differences in risk factors, comorbidities, and prehospital state (Table 5, see Materials and Methods). Each drug group was matched to participants from the non-treatment group (FIG. 4J) with comparable baseline characteristics (Table 5). The analysis showed shorter hospitalizations, lower respiratory intervention rates, and lower mortality rates in patients taking fibrates, statins, or IRE1α inhibitors. Fibrates users were also associated with lower ICU admission rates (Table 5). In contrast, patients taking SGLT2 inhibitors, metformin, or thiazolidinediones showed longer hospitalizations, higher mortality rates, and were associated with higher ICU admission rate and respiratory interventions (Table 5).

To study the effects of metabolic regulators on COVID-19 progression the dynamic changes in circulating C-reactive protein (CRP), neutrophils, and lymphocytes markers of SARS-CoV-2 induced immunoinflammatory response were analyzed. Dynamic changes during 21-day hospitalization were fitted using a locally weighted scatterplot smoothing (Lowess) comparing each drug group to its PSM-matched control (FIG. 5A-C). High CRP levels marking systemic inflammation gradually declined in all control groups over the duration of the study with no significant differences noted for patients taking statins, metformin, or SGLT-2 inhibitors. However, CRP levels in patients taking thiazolidinediones, that increase lipid synthesis, did not appear to decline. In contrast, patients taking fibrates or IRE1 inhibitors showed a significant decline in inflammation compared to their PSM-matched controls within 3 to 5-days of admission (FIG. 5A).

Neutrophil counts marking infection rose in all control groups to peak between days 10 to 14 (FIG. 5B). Treatment with IRE1 inhibitors, statins, metformin, or SGLT2 inhibitors showed lower initial neutrophil counts but relativity similar response to their PSM-matched controls, albeit with higher maxima for the SGLT2i group. Patients taking thiazolidinediones had significantly elevated neutrophil counts for the duration of hospitalization (FIG. 5B). However, patients taking fibrates showed consistently low neutrophil count throughout hospitalization.

Lymphocyte counts, a more complex marker of the immunoinflammatory response, drop during the first week of hospitalization below the lower limit of normal but are elevated during recovery in all PSM-matched control groups (FIG. 5C). Patients treated with statins, metformin, and thiazolidinediones showed lower lymphocyte count during the recovery period compared to their respective PSM-matched controls. However, patients taking fibrates or IRE1 inhibitors showed a significant but gradual elevation in lymphocytes, throughout hospitalization suggesting early recovery (FIG. 5C). Other blood measurements also confirm the effect of fibrates and to a lesser degree IRE1 inhibitors and the negative effects of thiazolidinediones on the severity and progression of COVID-19 (FIG. 5E).

COVID-19 severity was also investigated as a time-varying outcome in hazard risk using a Cox model accounting for baseline variance (FIG. 5D). Patient groups taking fibrates, statins, or IRE1 inhibitors were associated with lower mortality risk than their PSM-matched controls, showing an adjusted hazard ratio of 4×10⁻⁸, 0.79, and 0.65, respectively. Conversely, patients taking thiazolidinediones or metformin showed higher mortality risk with an adjusted hazard ratio of 1.3 and 1.4 compared to their PSM-matched controls, respectively (FIG. 5D).

Assessment of secondary outcomes of COVID-19 was carried out using a similar Cox model analysis looking at the incidence of mechanical ventilation, septic shock, acute liver injury, acute kidney injury, and acute cardiac injury. Patients taking fibrates or IRE1 inhibitors showed lower prevalence and hazard of all secondary outcomes compared to their respective PSM-matched controls, while those taking thiazolidinediones were associated with higher prevalence and hazard of all secondary outcomes (Table 4, FIG. 5F).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

The project leading to this application has received funding from the European Research Council (ERC) under the European Union's Horizon 2020 research and innovation programme (grant agreement No. [681870]).

1. A method of treating a coronavirus infection or preventing a symptomatic coronavirus infection in a subject in need thereof, the method comprising administering to said subject a therapeutic composition comprising at least one of a peroxisome proliferator-activated receptor alpha (PPARA) agonist and an inositol-requiring enzyme 1 (IRE1) pathway inhibitor, thereby treating a coronavirus infection or preventing a symptomatic coronavirus infection in a subject.

2. The method of claim 1, wherein said coronavirus is from the genus Betacoronavirus.

3. The method of claim 1, wherein said coronavirus is for the subgenus Sarbecovirus.

4. The method of claim 1, wherein said coronavirus is selected from Severe Acute Respiratory Syndrome (SARS)-CoV-1, Middle East Respiratory Syndrome (MERS) and SARS-CoV-2.

5. The method of claim 4, wherein said coronavirus is SARS-CoV-2.

6. The method of claim 1, wherein said subject is a mammal.

7. The method of claim 1, wherein said administering is within 1 day of diagnosis of said infection.

8. The method of claim 1, wherein said subject has not yet reached a cytokine storm stage of said infection.

9. The method of claim 1, wherein said subject is not currently or was not previously treated with a PPARA agonist or IRE1 pathway inhibitor.

10. The method of claim 1, wherein said subject does not suffer from a metabolic disease or disorder.

11. The method of claim 1, wherein said PPARA agonist produces at least a 10-fold greater agonizing effect on PPARA than on PPAR gamma.

12. The method of claim 1, wherein said PPARA agonist is selected from a fibrate, pirinixic acid and conjugated linoleic acid (CLA) and derivatives thereof.

13. The method claim 12, wherein said CLA is selected from 9-CLA and 10-CLA.

14. The method claim 12, wherein said fibrate is selected from aluminum clofibrate, bezafibrate, ciprofibrate, choline fenofibrate, clinofibrate, clofibrate, clofibride, fenofibrate, gemfibrozil, pemafibrate, fenofibric acid, ronifibrate and simfibrate.

15. The method claim 14, wherein said fibrate is fenofibrate.

16. The method of claim 1, wherein said IRE1 pathway inhibitor is an IRE1 alpha (IRE1α) inhibitor.

17. The method claim 16, wherein said IRE1 a inhibitor is selected from telmisartan, Sunitinib, STF-083010, 4 μ8C, KIRA6m, Kira8, Kira7, MKC8866, GSK2850163, Toyocamycin, APY29, MKC3946, MKC9989, NSC95682, B-I09, 3,6-DMAD, and IRE1α kinase-IN-2.

18. The method of claim 1, wherein said administering is oral administering.

19. The method claim 18, wherein said PPARA agonist or IRE1 pathway inhibitor is formulated to reach a Cmax in said subject within 1 day from administration.

20. The method claim 19, wherein said PPARA agonist is formulated as a fenofibrate nanocrystal, optionally wherein said fenofibrate nanocrystal is selected from Tricor® and Triglide®.

21. The method of claim 1, wherein said administering is intravenous administering.

22. The method of claim 1, wherein said PPARA agonist or IRE1 pathway inhibitor is administered on the first day of administration at twice a dose administered for treating a metabolic condition and is subsequently administered at said dose for treating a metabolic condition.

23. The method of claim 1, wherein said treating comprises at least one of reduced phospholipid accumulation in lung cells, reduced viral load, reduced symptoms, reduced inflammation, reduced risk of invasive mechanical ventilation, reduced risk of septic shock, reduced risk of acute liver injury, reduced risk of acute kidney injury, reduced risk of acute cardiac injury, reduced risk of ICU admission, reduced hospitalization time, reduced risk of Acute respiratory distress syndrome (ARDS), reduced risk of a cytokine storm and reduced risk of death.

24. The method claim 23, wherein said reduced inflammation is characterized by reduced levels of C-reactive protein (CRP).

25. The method of claim 1, wherein said treating occurs within 5 days of administering.

26. The method of claim 1, wherein said treating comprises treatment of post-acute sequelae of said coronavirus infection.

27. A therapeutic composition comprising at least one of a peroxisome proliferator-activated receptor alpha (PPARA) agonist and an inositol-requiring enzyme 1 (IRE1) pathway inhibitor for use in treating a coronavirus infection or preventing a symptomatic coronavirus infection in a subject in need thereof.


SEQUENCE LISTING (3 sequences)

SEQ ID NO: 1 - DNA, 20 bp, synthetic construct
  misc_feature 1..20: note = Synthetic
  source 1..20: mol_type = other DNA
tggggytttacrggtaacct

SEQ ID NO: 2 - DNA, 21 bp, synthetic construct
  misc_feature 1..21: note = Synthetic
  source 1..21: mol_type = other DNA
aacrcgcttaacaaagcactc

SEQ ID NO: 3 - DNA, 24 bp, synthetic construct
  misc_feature 1..24: note = Synthetic
  source 1..24: mol_type = other DNA
tagttgtgatgcwatcatgactag